(19) United States
(12) Patent Application Publication
(10) Pub. No.: US 2004/0208846 A1
(43) Pub. Date: Oct. 21, 2004

(54) Mini-Ad vector for immunization

(76) Inventors: Wei-Wei Zhang, Libertyville, IL (US); Ramon Alemany, Grayslake, IL (US); Yifan Dai, Grayslake, IL (US); Steven Josephs, Grayslake, IL (US); Cristina Balague, Grayslake, IL (US); David Ayares, Blacksburgh, VA (US); Richard Schneiderman, Highland Park, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 09/837,079

(22) Filed: Jun. 15, 2004

Related U.S. Application Data
(63) Continuation-in-part of application No. 08/658,961, filed on May 31, 1996, now abandoned.
(63) Continuation-in-part of application No. 08/791,218, filed on Jan. 31, 1997, now abandoned.
(60) Provisional application No. 60/197,734, filed on Apr. 18, 2000.
(60) Provisional application No. 60/198,501, filed on Apr. 18, 2000.

(51) Int. Cl. A61K 48/00

(52) U.S. Cl. 424/93.2; 514/44

(57) ABSTRACT

The present invention provides a method for treating a disorder such as hemophilia. A method of treating hemophilia in a mammal by administering recombinant virus virions comprising a nucleotide sequence having an adenoviral inverted terminal repeat fusion sequence, a packaging signal, a transcriptional control region, and a nucleic acid encoding a therapeutic protein such as FVIII. In addition, the DNA molecule does not encode an adenoviral protein. It is preferred that the virions be administered to the mammal under conditions that result in the expression of the therapeutic protein at a level that provides a therapeutic effect in said mammal.

This application claims priority to U.S. application Ser. No. 08/658,961 filed on May 31, 1996; U.S. application Ser. No. 08/791,218 filed on Jan. 31, 1997; U.S. provisional application No. 60/197,734 filed Apr. 18, 2000; and, U.S. provisional application No. 60/198,501 filed Apr. 18, 2000.

### FIELD OF THE INVENTION

The present invention relates to adenoviral vectors for delivery of nucleic acids for expression of proteins, peptides and the like in cells. In particular, the adenoviral vectors are largely or completely devoid of adenoviral protein coding sequences.

### BACKGROUND

An important issue in the development of genetic medicine is the development of preferred gene delivery systems. The preferred system of gene delivery must possess several properties that are currently unavailable in a single gene therapy vector. The preferred vector must retain adequate capacity to accommodate large or multiple transgenes including regulatory elements and be amenable to simple manipulation and scale-up for manufacturing. Such a vector must also be safe and demonstrate low toxicity as well as demonstrate highly efficient and selective delivery of transgenes into target cells or tissues. Finally, such a vector must be capable of supporting appropriate retention, expression, and regulation of the transgenes in target cells. The present invention encompasses a novel design of a high-capacity and highly-efficient Ad vector system and is focused on resolving the issues and concerns of those skilled in the art regarding an preferred gene delivery system.

An exemplary condition treatable with vectors such as those described herein is Hemophilia A, which results from deficiencies in expression or function of clotting factor VIII (FVIII). Treatment of hemophilia currently involves infusion of normal FVIII protein obtained from plasma concentrates or as purified from cultured cells engineered to express recombinant FVIII protein (1). Therapeutic benefit is achieved by restoration of plasma levels to 5-10% of normal plasma levels (200-300 ng or 1 unit per milliliter; Ref. 2). Studies have shown that maintenance of greater than 30% of the normal plasma levels allows for a near normal lifestyle (3). Gene therapeutic approaches towards treatment of hemophilia have exciting potential; however, several major challenges remain to be overcome for these treatment modalities to become reality (4). The present invention provides several tools with which these difficulties may be resolved.

FVIII is normally produced in the liver and is comprised of heavy chain polypeptides with a range of apparent molecular weights of from 92 kDa to 210 kDa derived from the amino terminus of the nascent polypeptide and a C-terminal light chain of 80 kDa (53). It is protected from proteolysis by formation of a complex with von Willebrand's factor (vWF). The activated form functions in the blood clotting cascade as a cofactor along with activated factor IX (FIXa), negatively charged phospholipids and calcium ions to convert factor X to its activated form, Xa. The human cDNA is 9 kb in length and encodes a polypeptide of 2351 amino acids comprised of several domains in the order A1, A2, B, A3, C1 and C2 (5-7). The A and C domains are critical for functional activity whereas the majority of the B domain, consisting of approximately 980 amino acids, is dispensable for activity (8). Since the full-length FVIII cDNA exceeds the size limitations of retrovirus and adenovirus vectors, most gene therapy protocols utilized by those skilled in the art to date have utilized a FVIII cDNA having the B domain deleted, such that the remaining cDNA is approximately 4.5 kb.

Retroviral vectors were among the first to be studied for use in gene therapy (64, 65). The size capacity for insertion of exogenous DNA is limited to approximately 7.5 kb. Generally, it has been difficult to obtain high-level expression of FVIII from retroviral vectors due to problems of viral mRNA instability and difficulties of expression of the mRNA encoding the FVIII gene product (62, 63, 74). Additionally, infection of non-dividing cells, such as the majority of the liver cells, is also problematic. One way to overcome this limitation has been to perform a ⅔ partial hepatectomy prior to retrovirus infection to allow infection of actively regenerating liver cells (73). In another approach, long-term expression has been achieved using muscle specific enhancers but only low levels of gene product, FIX, were achieved (78). Therapeutic levels of FVIII have been achieved in mice (77).

Recently, an E1-substituted adenoviral vector comprising a B-domain deleted FVIII cDNA under control of the murine albumin promoter has been utilized to achieve therapeutic levels of human FVIII expression in mice and dogs (13-15). Upon administration of high doses (4×10⁹ pfu) of this adenoviral vector comprising the FVIII cDNA, gene expression in immunocompetent animals was limited in duration; a gradual decline in gene expression correlated with a loss of detection of the adenoviral vector DNA in liver tissue (13). The decline of expression was partially overcome by lowering the vector inoculum resulting in therapeutic plasma levels of FVIII for 22 weeks following administration (16). However, the C57Bl/6 strain of mice used in those studies exhibits an attenuated immune response; these results, then, may not reflect those that may be obtained using fully immunocompetent animals. The rapid decline of expression using these vectors in dogs could be attributed to immune responses to the human FVIII protein or adenovirus proteins. To ascertain the potential use of such E1-substituted adenovirus vectors in humans, experiments with larger animals such as dogs using canine FVIII may be required.

Adenoviral vectors may be preferred for delivery of FVIII due to the fact that: 1) intravenous (I.V.) injection of adenovirus results in targeted gene expression to the liver, in part due the accumulation of the adenoviral vector primarily in liver tissue; 2) expression of FVIII from the liver has resulted in a significant elevation of levels of FVIII in plasma; and, 3) the liver is a major site of synthesis of FVIII in normal individuals.

Significant difficulties, however, are associated with adenoviral gene delivery. For instance, Ad does not normally integrate into the host cell genome. To sustain long-term transgene expression, an Ad vector must include the elements required for host cell integration or other mechanisms of DNA retention. Additionally, the immune response mediated against the adenoviral vector makes re-administration of the vector very difficult (76). The mini-Ad vector of the present invention has eliminated all adenovirus genes from the mini-Ad vector carrying the transgene. This at least partially eliminating any detrimental immune response that may be raised by Ad gene expression in the host cell, which may contribute to the decline of transgene expression.

An adenoviral vector that allows for a large heterologous DNA insert has been described in international patent application WO 96/33280 (see ref. 132). This vector, however, does not provide elements for integration into the target cell genome or for episomal maintenance of the vector upon entry into a target cell. The present invention provides elements that allow for retention of the delivered transgene in the host cell, either by integration into the target cell genome or by maintenance as an episomal nucleic acid. One method with which this is accomplished by the present invention includes facilitation of integration of the transgene into the host cell genome using viral integration mechanisms. The adeno-associated virus (AAV) genome has the capability of integrating into the DNA of infected cells and is the only example of an exogenous DNA that integrates at a specific site, AAVS1 at 19q13.3-qter, in the human genome (35, 133). The minimal elements for AAV integration are the inverted terminal repeat (ITR) sequences and a functional Rep 78/68 protein. The present invention incorporates these integration elements for integration of the transgene into the host cell genome for sustained transgene expression. The present invention also provides an adenoviral vector capable of homologous recombination into the genome of a target cell, another significant advantage over adenoviral vectors currently available to one skilled in the art. The present invention also provides elements that allow episomal replication of the transgene.

In vitro model systems have been developed to detect site-specific integration of AAV or AAV-based vectors in immortalized cell lines (22-24, 134), in episomal systems (25-27), and in cell-free extracts (28). A comparison of transduction efficiencies of AAV using either primary human cells or immortalized cell lines, demonstrated that the transduction efficiency was 10-60 times greater in immortalized human cells than in primary cells (29). These results stress the importance of using primary cells, or even better, in vivo model systems, to accurately evaluate AAV vectors for gene therapy applications. However, to date, no in vivo animal model system has been developed to detect site-specific integration. An animal model having the human AAVS1 sequence incorporated into its genome is provided in the present invention. This animal model will be useful for evaluation of vectors containing the AAV integration mechanism, not only to test site-specific integration, but also in vivo gene delivery, gene transduction efficiency, tissue distribution, and duration of gene expression.

Animal models, including hemophiliac dogs and mice, have been used to test efficacy of FVIII preparations (11, 13-15, 82, 83). However, for human FVIII gene therapy, an immune response against the human FVIII protein in an animal model may complicate studies of long-term delivery of FVIII. This invention provides an animal model that is tolerized to human FVIII. The lack of an immune response to human FVIII in such animals allows for accurate evaluation of transgene delivery without immunological complications that may arise due to an immune response against human FVIII.

The mini-Ad vector system of the present invention was developed based on two major findings: 1) the discovery of an Ad-SV40 hybrid (17) in which the majority of the viral genome was replaced by SV40 sequences but was able to be processed and packaged due to the presence of Ad ITR and packaging elements; and, 2) that Ad packaging may be attenuated by partial deletion of the packaging signal (18). Other adenoviral vector packaging systems based on incorporation of minimal cis elements for packaging and genome replication are under development by others (19-21).

### SUMMARY OF THE INVENTION

The present invention provides a method for treating a disorder such as hemophilia. A method of treating hemophilia in a mammal by administering recombinant virus virions comprising a nucleotide sequence having an adenoviral inverted terminal repeat fusion sequence, a packaging signal, a transcriptional control region, and a nucleic acid encoding a therapeutic protein such as FVIII. In addition, the DNA molecule does not encode an adenoviral protein. It is preferred that the virions be administered to the mammal under conditions that result in the expression of the therapeutic protein at a level that provides a therapeutic effect in said mammal.

### BRIEF DESCRIPTION OF THE FIGURES

FIG. 1. The principle of the mini-Ad vector system. Shown are three of the major components of the mini-Ad vector system: the helper Ad, the mini-Ad vector, and the Ad helper cell. E1 supplied by the helper cells allows the helper Ad to replicate itself and synthesize the late viral proteins that form the viral capsids. The packaging of the helper Ad genome into the capsid is inefficient as the packaging signal of the helper Ad of the present invention has been attenuated. In the presence of the mini-Ad vector genome, the helper Ad supports replication of the mini-Ad vector genome, which is preferentially packaged because its wild-type packaging signal has high affinity for the helper virus packaging proteins (31). Further purification of the mini-Ad vector may be achieved using a biochemical or physical method, such as ultracentrifugation.

FIG. 2. Comparison of the current Ad vectors with the present invention. Depicted are the general structures and complementary mechanisms of the current Ad vectors compared to those of the mini-Ad vector system.

FIG. 3. The prototype of the helper virus and the mini-Ad vector. A. The placement of the packaging signal in reference to the left ITR of the adenovirus. B. The sequence of the packaging signal region of the wild type adenovirus 5 is shown. The brackets indicate the regions deleted to comprise the attenuated helper virus packaging signal. C. The repeated region of the sequence shown in B are listed along with the consensus repeat. D. Additional embodiments of Mini-Ad vectors.

FIG. 4. Construction of the shuttle vector to generate the packaging attenuated helper AdHβ. To construct GT5000, the mutant packaging signal sequence, mt Ψ, was amplified by PCR and substituted in a shuttle vector with an Ad5 sequence extended to 28.9 mu (GT4004). A β-gal expression cassette from pTk-β was cloned into the E1-deletion of GT5000 to give the shuttle vector GT5001.

FIG. 5. Generation of AdHβ. The shuttle vector GT5001 (see FIG. 8) was cotransfected with pJM17 in 293 cells. Recombination in the homologous 7 Kb region between these plasmids (9.24 to 28.9 mu of Ad5) yields a packageable virus with the left arm derived from GT5001. The numbers in the left region of AdHβ shown at the bottom correspond to Ad5 nt sequence and indicate extension of the double deletion in the packaging signal as well as in E1 where the β-gal expression cassette is inserted. Two weeks after cotransfection, several blue plaques were isolated and the mutation in the packaging signal was analyzed by PCR with oligos 7 an 8. The size of the amplified fragment, 310 bp for the wild type packaging signal (wt Ψ) and 177 bp for the mutant packaging signal (mt Ψ), can be distinguished in a 2% agarose gel as shown. 1 Kb ladder, 1 Kb DNA ladder marker from Gibco BRL (Gaithersburg, Md.); wt Ψ and mt Ψ PCR controls where vDNA was extracted from an E1-deleted vector, Ad-CMVβgal, and from dl10/28 virus respectively; blue plaque, vDNA from a plaque that stained blue with X-gal; white plaque, vDNA from a plaque that did not stained blue with X-gal (see FIG. 6).

FIG. 6. Amplification and characterization of AdHβ. The packaging signal of AdHβ was amplified after every passage due to the possibility that recombination with the endogenous left Ad5 sequences present in 293 cells could generate a replication competent adenovirus (RCA, E1+) or an E1− adenovirus with wt Ψ. Wt Ψ was not detected in the passages previous to the CsCl purification (4 to 8). At passage 9, when AdHβ was purified, the viral DNA content was analyzed separately for every of the five bands of the gradient. In a 1% agarose gel (bottom left), almost no vDNA is observed in the upper three bands, indicating that they are formed mostly by empty capsids. Lower bands (4 and 5) are formed by full capsids. By PCR the expected mutant packaging signal is detected in all the bands (bottom right). 1 Kb ladder marker (as in FIG. 9) at the left lane of every gel. Gel with vDNAs also contains 1/Hind III marker.

FIG. 7. Construction of mini-viral plasmids. These plasmids are constructed to determine the effect of various deletions of the adenoviral genome on packaging when complemented with AdHβ. All constructs contain the green fluorescence protein cDNA (GFP, striped box) driven by the CMV promoter with a β-actin enhancer (thick arrow). M7.9 (bottom right) also has the neomycin cDNA and an internal ribosome entry site (IRES). The top six are derived from M32, which is a pJM17 derivative with a 10 Kb deletion in the middle of the Ad5 genome. The bottom two are derived from pBluescript-KS (Stratagene, CA) with the minimal cis elements for replication and packaging of Ad5. Numbers correspond to Ad5 map units and indicate the deletion and insertion sites. 0/100 or 100/0 indicates the natural fusion of the inverted terminal repeats (ITR) of Ad5 DNA, Ad5 DNA, thick lane; plasmid backbone DNA, thin lane.

FIG. 8. Schematic representation of the mini-adenoviral (mini-Ad) vectors constructed for packaging. At the top, the Ad5 transcription map and map units (mu) with the early (E) and late (L) transcription regions. MLP/TL: major late promoter and tripartite leader. The inverted terminal repeats (ITRs) and the packaging signal (Ψ) are the unique common sequences in all the miniAd vectors. The vectors are shown in a linear form as found after replication and in the capsid. The circular plasmids used for vector generation contain the same sequences but fused head-to-tail by the ITRs. Every miniAd vector name refers to its size in Kb. M32 to M20 derive from pJM17 by progressive deletion of the central adenovirus genome. In these vectors the plasmid backbone (pBRX, not drawn) is located at 3.7 mu. M6.5 to vGnE5E3 are constructed in the pBluescript backbone (not drawn; located before the GFP-expression cassette) by insertion of neomycin cDNA and human genomic fragments from chr. 4q11-22.

FIG. 9. Two methods of complementation. To generate the mini-viral vectors two separate complementation protocols were used that gave similar yields. In the first method, the mini-Ad plasmid is cotransfected with viral DNA from AdHβ, and the cells are cultured until a CPE is observed. In the second method, three days after an initial cotransfection of the mini-Ad plasmid with pBHG10, AdHβ is added as virus, and cells are cultured until a CPE is observed.

FIG. 10. Packaging efficiency of mini-viral vectors of different sizes. Packaging efficiency and amplification of miniAd vectors of different sizes. The virus produced after co-transfection is indicated as passage 0. The crude lysate was used to infect 293 cells to produce passage 1. 1 ml of crude lysate passage 1 was used to infect 106 293 cells and 24 h later the number of fluorescent cells were counted (transducing units/ml, dark columns). 24 h later CPE appeared and virus was extracted by freeze/thaw (crude lysate passage 2). Again, 1 ml of crude lysate passage 2 was used to infect 106 293 cells and 24 h later the number of fluorescent cells were counted (striped columns). The difference between passage 1 and 2 indicates an amplification yield of 5 and the difference between the miniAd vectors indicates the effect of the size in the packaging.

FIG. 11. Purification scheme of M32. After amplification of M32 through several passages, the crude extract was CsCl-separated. The first gradient resulted in four bands: three upper and one lower. M32 and AdHβ co-purify in the band of higher density (number 4 or lower). These were collected separately, dialyzed and used to infect 293 cells as shown in FIG. 12. Fractions were collected from second separated gradients of the upper and the lower bands, and used to infect 293 cells as shown in FIG. 16.

FIG. 12. Ad5 packaging signal modification with GAL4 binding sites. The nucleotide sequences between the Xho I and Xba I sites are shown (design #1 and design #2). In design #1, there are two GAL4 binding sites before A repeat I and one GAL4 binding site between A repeat II and VI. In design #2, the two GAL4 binding sites before A repeat I and two GAL4 binding sites after A repeat VII. The sequences underlined are 17 mer GAL4 binding sites. The sequences in italics are A repeats. The distance between center of each GAL4 binding site and A repeats is indicated.

FIG. 13. Ad5 packaging signal modification with tetO sequence. The nucleotide sequences between the Xho I and Xba I sites are shown (design #1 and design #2). In design #1, there are two tetO sequences before A repeat I and one tetO sequence between A repeat II and VI. In design #2, two tetO sequences are present before A repeat I and further tetO sequences after A repeat VII. The sequences underlined are 19 mer tetO sequence. The sequences in italics are A repeats. The distance between the center of each tetO binding site and the A repeats is indicated.

FIG. 14. Position and sequences of synthetic oligos for Ad Pac⁻-GAL4 modification. Gal#1 to Gal#8 are synthetics oligos flanking the sequence between the Xho I and Xba I sites in design #1 and #2. The position and direction of each oligo is indicated by arrow bar. The sequences of Gal#1 to Gal#8 are listed.

FIG. 15. Position and sequences of synthetic oligos for ad Pac⁻-tetO modification. tet#1 to tet#10 are synthetics oligos to cover the sequence between the Xho I and Xba I sites in design #1 and #2. The position and direction of each oligo is indicated by arrow bar. The sequences of tet#1 to tet#10 are listed.

FIG. 16. Construction of CMV-E1 mammalian expression vector. Adenovirus 5 sequences 462-3537 (AflIII-AflII Fragment) coding for E1A and E1B were blunt-end cloned into the EcoRV site of pcDNA3.

FIG. 17. Southern blot analysis of G418^{r} A549E1 clones. Genomic DNA was digested with Hind III and probed with a 750 bp E1 probe (PstI fragment). Lane 1: 1 kb DNA ladder; Lane 2: A549; Lane 3: 293; Lane 4: A549E1-68; Lane 5: Subclone A549E1-68.3.

FIG. 18. Analysis of the E1 protein expression in transformed cell lines. A. Western blot analysis of E1A protein expression in A549 cells (Lane 1), 293 cells (Lane 2), and A549E1-68 (Lane 3), using an E1A-specific monoclonal antibody (M73, Oncogene Science). B. Metabolic ³⁵S labeling and immunoprecipitation of E1B proteins in A549 cells (Lane 1), 293 cells (Lane 2), and A549E1-68 (Lane 3), using E1B p55-specific monoclonal antibody (Oncogene Science).

FIG. 19. Schematic of pAlb12.5CAT plasmid. The plasmid comprises the pAlb12.5CAT 12.5 kb human albumin promoter (EcoRI to HindIII) operably linked upstream of the chloramphenicol acetyl transferase gene (CAT) in the pBRCAT plasmid vector. The proximal promoter, and the enhancer regions (E_{1.7} and E₆) are shown.

FIG. 20. Cloning of the 12.5 kb human albumin promoter into pBlueScript KS⁺ vector. The EcoRI to AvaI 10.5 kb fragment and the 2.0 kb AvaI to HindIII albumin promoter fragments were separately isolated from pAlb12.5CAT and simultaneously ligated into the EcoRI/HindIII site of pBlueScript-KS⁺ vector to generate GT4031.

FIG. 21. Cloning of the hFVIII expression cassette into GT4031. The 7.5 kb human FVIII cassette was excised from plasmid GT2051 using XhoI and SalI and ligated into SalI site of GT4031 resulting in GT2053, comprising the human albumin promoter operably linked to the hFVIII cDNA.

FIG. 22. Construction of an albumin promoter-FVIII minivirus plasmid. A fragment comprising the adenovirus 5′ ITR and packaging signal from GT2033 was excised using XhoI and cloned into the SalI site of GT2053. Plasmids having either the forward or reverse orientation (GT2061 and GT2059, respectively) of the ITR were obtained. The insert in GT2061 is oriented with the unique SalI site proximal to the FVIII gene. GT2059 contains the Ad ITRs in the opposite orientation of GT2061.

FIG. 23. Restriction digest profiles of GT2053, GT2059 and GT2061. Digests show the expected banding patterns for BamHI, XbaI, ClaI and XhoI in combination with SalI.

FIG. 24. Diagram of the albumin/α-fetoprotein gene region on chromosome 4. The diagram illustrates three regions that may serve as 3′ recombination arms for homologous recombination: 1.) Alb-E5, the 3′ region of the albumin gene; 2.) AFP-3, a central region of the α-fetoprotein gene; and, 3.) EBB14, located further 3′ in the α-fetoprotein gene.

FIG. 25. Cloning scheme. Restriction enzyme maps of three vectors (A. B, and C) comprising distinct 3′ homologous recombination arms after cloning the arms into GT2061 (illustrated above panels A, B, and C).

FIG. 26. Cloning scheme. Detailed cloning scheme for GT2063 where the 3′ 6.8 Kb XhoI fragment of the human albumin gene of clone pAlb-E5 was cloned into the SalI site of GT2061. A minivirus based on this vector is a potential in vivo therapeutic tool for FVIII gene therapy.

FIG. 27. Restriction enzyme mapping. Agarose gel demonstrating restriction enzyme digestion of the vectors utilized in the generation of the plasmid comprising the albumin promoter-driven hFVIII with the 3′ albumin homologous recombination arm as shown in FIG. 9. EcoRI and ClaI digests are shown for each of the indicated constructs.

FIG. 28. Scheme for generating the mini-AdFVIII virus. Shown are two schemes (A and B) for generating a hFVIII minivirus. Scheme A: helper virus genomic DNA and plasmid GT2063 were cotransfected by calcium phosphate precipitation into 293 cells (ATCC# CRL 1573) on day 1. Transfection was by calcium phosphate precipitation. At day 6, cytopathic effect (CPE) was observed and cell lysates prepared. Lysates were subsequently harvested every three days following infection until passage 4, at which time the virus preparation was amplified five-fold. Media was changed daily following infection. Scheme B: plasmid pBHG10 was co-transfected with plasmid GT2063 using Lipofectamine (Gibco) into 293 cells. Infection with helper Ad was performed 72 hours later at a multiplicity of 5 pfu/cell. After five days, CPE was observed and cell lysates prepared. Infections were subsequently performed using equivalent inoculum volumes until passage 3 at which time the virus can be amplified 5 fold. Media was changed daily following infection.

FIG. 29. Generation of a mini-Ad vector containing FVIII. The mini-AdFVIII vector was generated by transfection of the mini-Ad plasmid GT2063 in 293 cells and infection with the helper AdHβ. When the cytopathic effect (CPE) was complete, the cells and supernatant were collected (passage 0). For propagation, virus was extracted from the cells by several freeze/thaw cycles and utilized to infect fresh 293 cells. At each passage, 740 μl of supernatant was used to extract viral DNA by incubation in a solution comprising SDS, EDTA, and Proteinase K followed by ethanol precipitation. Previous to the virion lysis step, supernatants were subjected to DNase I digestion to avoid contamination from GT2063 plasmid DNA. One twentieth of the purified viral DNA (vDNA) was utilized as substrate in amplification of the packaging signal sequence by polymerase chain reaction (PCR). The expected size for the FVIII mini-Ad and helper amplified regions were 177 and 310 bp, respectively. DNA size marker: 1 Kb ladder from Gibco (Gaithersburg, Md.). Supernatants from untransfected 293 cells with or without additional GT2063 plasmid were used as negative control and as a control of DNase I treatment, respectively.

FIG. 30. Southern blot analysis of vDNA from passages 0 to 21 of the mini-AdFIII vector. vDNA was purified as in FIG. 14. One half of the purified vDNA (corresponding to 370 μl of supernatant) was digested with Pst I, separated on a 1% agarose gel, and blotted to a nylon membrane. A probe corresponding to sequence adjacent to the right (3′) ITR present in both the mini-Ad and the helper was utilized to detect the vDNA. Expected size of detected fragments: mini-AdFVIII=3.3 Kb; AdHβ=2.2 Kb. Four independent blots are shown (A. B, C and D). Specific hybridization to marker fragments was utilized for normalization.

FIG. 31. Dynamic fluctuation in mini-AdFVIII and helper over time during serial passage. The plot was obtained by densitometrical quantification of the bands shown in FIG. 15. Helper is labeled by the clear line with squares; mini-AdFVIII is labeled by the dark line with diamonds. One unit is defined on the Y axis as the lowest amount detected (corresponding to the quantity of helper vDNA at passage 18). Other values are normalized to that unit.

FIG. 32. Separation of mini-AdFVIII and AdHβ by CsCl gradient centrifugation. The bottom band from the first gradient contained virions that were further separated by application to a second gradient. The different sizes of the mini-Ad (31 kb) and the helper (37.1 kb) allowed separation that resulted in the generation of an upper fraction having a 10:1 mini-Ad/helper virus ratio and a lower fraction having a 1:10 mini-Ad/helper virus ratio as determined by Southern blot.

FIG. 33. MiniAd FVIII-mediated expression of human Factor VIII in vivo. A. Expression of human Factor VIII in hemophilic mice injected with 2×10¹¹ vp of MiniAdFVIII as measured by ELISA; representative individuals are shown. B. Dose-response study in C57BL/6 mice; three individuals per dose were used and Factor VIII expression was measured by ELISA; Mean±SD is depicted.

FIG. 34. Map of clone GT2074. The plasmid comprises an expression cassette (comprising the elongation factor-1 (EF-1; ref. 52) promoter operably linked to the B-domain deleted human FVIII cDNA) excised from plasmid GT4020 by SalI digestion cloned into the unique Sal I site of GT2073. The 3′ proximal albumin promoter region downstream of the Pme I site in pALB12.5 including the TATA and CCAAT (32) were deleted. The expression cassette contains the elongation factor I promoter linked to the B-domain deleted human FVIII cDNA.

FIG. 35. Map of pCMV-hFVIII. This plasmid comprises CMV promoter operably linked to the full-length hFVIII coding region as cloned into the Sal I site of GT2073. The cytomegalovirus promoter was derived from pCMVβ (Clontech, Palo Alto, Calif.).

FIG. 36. Schematic representation of the plasmids used to test for integration frequency and specificity. Plasmids GT9003 and GT9004 contain a neo expression cassette flanked on both sides by AAV ITR sequence; GT9012 and GT9013 contain a GFP expression cassette flanked by AAV ITR sequence; GT9003 and GT9012 also contain a Rep78 expression cassette upstream of the integration cassette. To construct plasmid GT9003, Rep sequences from 193 to 2216 in the AAV genome were amplified by PCR (Pfu pol) from plasmid pSUB201, and cloned into pCRII (Invitrogen, CA). The resulting plasmid (GT9000) was digested with NotI and XhoI and a fragment containing an SV40 polyA site (Not-Sal I) was cloned in those sites. The resulting plasmid (GT9001) was digested with XbaI and blunt-ended with Klenow. A PvuII-PvuII fragment containing the whole AAV genome was obtained from pSUB201 and subcloned in the blunted XbaI site in GT9001. This plasmid (GT9002) was then cleaved with XbaI which removes the AAV coding sequences leaving the AAV ITRs. A neo-expression cassette (BamHI-BamHI) was then subcloned into GT9002 using XbaI and BamHI adaptors, giving rise to plasmid GT9003. Plasmid GT9004 was generated by removing the Rep coding sequences GT9003 using EcoRI. Plasmid GT9012 and GT9013 were generated by replacing the neo sequences (XbaI-XbaI) in GT9003 and GT9004, respectively, with a GFP expression cassette (SpeI-NheI).

FIG. 37. Design of the mini-adenoviral vector containing an integratable GFP cassette. Minimal Ad elements necessary for replication and packaging present in the construct are the Ad ITRs and packaging signal. The GFP cassette is contained between two AAV ITRs. A Rep expression cassette is positioned outside the integratable segment. Rep expression can be regulated by the Tet operator in a cell line stably expressing the repressor tet-KRAB. In the target cells, the expression of Rep should provide targeted integration of the sequences flanked by AAV ITRs in the AAVS1 site in chromosome 19.

FIG. 38. Immunoprecipitation of Rep proteins in 293 and Chang liver cells. Cells grown in 10-cm Petri dishes were transfected with 10 mg of plasmids GT9001, GT9003, and GT9004 (see FIG. 31 for details on construction of plasmids). Untransfected and GT9004-transfected cells were used as negative controls. Two days after transfection, cells were lysed and Rep proteins were immunoprecipitated using an anti-Rep monoclonal antibody (clone 226.7; ARP, Belmont, Mass. 02178) coupled to protein G-agarose, run on a 10% polyacrylamide gel and immunoblotted with the same antibody used in immunoprecipitation. Proteins were visualized by chemoluminiscence (ECL kit, Amersham). The migration of Rep78 and Rep52 proteins is indicated.

FIG. 39. Southern blot of 293 clones transfected with plasmids GT9003 or GT9004. Fifteen mg of genomic DNA from several neo-resistant clones as well as a neo-resistant population (indicated as pool) were digested with EcoRI, electrophoresed, blotted onto a nylon membrane (Hybond-N, Amersham) and hybridized to an AAVS1 probe, spanning an 8 Kb EcoRI-EcoRI fragment. The normal AAVS1 locus is indicated (panel A). Some GT9003 clones show a shifted band corresponding to disruption of one of the AAVS1 loci. Panel B shows the same membrane rehybridized to neo sequences.

FIG. 40. Southern blot of 293 clones transfected with plasmids GT9012 or GT9013. The conditions are as described in FIG. 33. A) AAVS1 Southern blot. Some clones derived from plasmid GT9012 show rearrangements of AAVS1. B) Same blot rehybridized to GFP sequences.

FIG. 41. Southern Blot Analysis of AAVS1 P1 Genomic Clones. Plasmid DNA (1 ug) isolated from four P1 genomic clones (termed P1 clone 6576, P1 clone 6577, P1 clone 6578, and P1 clone 6579) in which the AAVS1 sequence was detected by PCR (AAVS1 PCR(+)) digested with EcoRI (FIG. 23A) or EcoRI in combination with EcoRV electrophoresed on a 1% agarose gel, blotted onto a nylon membrane (Hybond N+, Amersham), and hybridized using the 253 bp AAVS1 PCR product as a probe. In both A and B, Lane 1 represents P1 clone 6576, Lane 2 represents P1 clone 6577, Lane 3 represents P1 clone 6578, and Lane 4 represents P1 clone 6579.

FIG. 42. Construction of the pAAVS1-Neo Vector. An 8.2 kb EcoRI fragment comprising an AAVS1 integration sequence was isolated from P1 clone 6576 and ligated into the EcoRI site of the Neo expression vector, pGKneo, to create pAAVS1-Neo.

FIG. 43. Generation Of Transgenic Mice Harboring The Human AAVS1 Integration Sequence. Diagrammed are the sequences of steps involved in the generation of a transgenic mouse. ES cells were transfected with the AAVS1 plasmid clone, microinjected into blastocysts which are then implanted into foster mothers. After approximately 17 days, chimeric mice were cross-bred to C57BL/6 mice and progeny tested for the presence of the AAVS1 transgene. Cross-breeding of the positive progeny was performed to generate a line that is homozygous for the transgene. These models are then utilized for testing in vivo delivery of the mini-Ad vectors modified to comprise the adeno-associated virus integration system to evaluate the efficiency of site-specific integration of the vector DNA.

FIG. 44. PCR Analysis of Neo^{R} ES Cell Clones Following Transfection With pAAVS1-Neo. Genomic DNA independently isolated from 17 Neo^{R} ES cell clones (3.1-3.17) and from untransfected parental ES cells were screened by PCR using the AAVS1-specific primers U2493 and L2722 to confirm that the NeO^{R} clones harbored the AAVS1 sequence. The PCR reaction samples were loaded on a 1.5% agarose gel as follows Lane 1—1 kb DNA size markers (Gibco/BRL); Lane 2—pAAVS1-Neo plasmid control; Lane 3—Untransfected parental ES cell DNA; Lanes 4 through 20—DNA from 17 individual pAAVS1Neo-transfected, Neo^{R} ES cell clones.

FIG. 45. Southern Blot Analysis of AAVS1 PCR (+) ES Cell Clones. Genomic DNA from two AAVS1 PCR (+) ES cell clones (ES#4 and ES3.16) and from the parental ES cells was digested with EcoRI in combination EcoRV, electrophoresed on a 0.8% agarose gel, blotted onto Hybond N+ nylon membrane, and hybridized with an 8.2 kb AAVS1 probe. Lane 1—AAVS1 ES#4; Lane 2—AAVS1 ES#3.16; Lane 3—Parental ES Cells. Expected fragments resulting from integration of the entire AAVS1 sequence into the ES cell genome are 5.2 and 3.0 kb.

FIG. 46. PCR Analysis For Detection of the AAVS1 Transgene in Chimeric Mice. Genomic DNA was isolated from the tails of AAVS1 chimeras and, independently, from non-chimeric littermates, and screened by PCR using the AAVS1-specific primers U2492 and L2722. PCR reactions were loaded onto a 1.5% agarose gel as follows: Lane 1—1 kb DNA size markers; Lane 2—pAAVS1-Neo plasmid control (c); Lane 3—untransfected parental ES cell DNA; Lane 4—AAVS1 ES#4 ES cell DNA (4); Lane 5—Tail DNA from a non-chimeric littermate; Lane 6—Tail DNA from a low-percentage chimera (less than 10% agouti coat color); Lane 7—Tail DNA from a high-percentage AAVS1 chimera (greater than 90% agouti coat color). Expected PCR product=253 bp.

FIG. 47. Construction of the hFVIII mouse tolerization vector. The 7.2 kb full-length hFVIII gene was excised from GT2051 using Not I and cloned into the Not I site of GT2057 to generate a mAFP-FVIII cassette in GT2058. The mAFP-hFVIII cassette was then excised from GT2058 using Aat II and Sal I fragment and cloned into pGKNeo at Aat II/Xho I to create mAFP-hFVIII-pGKNeo (GT2062).

FIG. 48. Southern Blot Analysis of mAFP-hFVIII-pGKNeo ES cell clones. Genomic DNA from untransfected parental ES cells and from 4 Neo^{R} ES clones was digested with Xba I (see FIG. 29), electrophoresed, blotted, and hybridized using a full-length hFVIII Not I fragment as a probe. Expected fragment size indicating insertion of the mAFP-hFVIII cassette was 7.8 kb, as shown in the Figure.

FIG. 49. Construction of the mAFP-EGFP-1 vector. A 7.5 kb EcoRI/Sal I fragment containing the entire AFP promoter/enhancer region was cloned into the pEGFP-1 vector (Clontech) at EcoRI/Sal I to create pAFP-EGFP-1.

FIG. 50. Scheme for the production of a transgenic mouse tolerized in utero to hFVIII for testing in vivo delivery of hFVIII without immune consequences. Diagrammed are the sequence of steps involved in the generation of transgenic mice. ES cells transfected with the AFP-hFVIII-Neo plasmid containing the FVIII cDNA operatively linked to the a-fetoprotein promoter are microinjected into blastocysts which are then implanted into foster mothers. After approximately 17 days, chimeric mice are cross-bred to C57BL/6 mice and progeny are tested for the presence of the AFP-hFVIII transgene. Cross-breeding of the positive progeny was performed to generate a line which is homozygous for the transgene. These models are used for testing in vivo delivery of mini-Ad vectors modified to comprise the human FVIII expression cassettes.

FIG. 51. RIP-EGFP vector for use in the production of transgenic mice tolerized to Green Fluorescence Protein (GFP). Shown is a restriction enayme map of the RIP-pEGFP from the BS plasmid. This plasmid (4855 bp) comprises the green fluorescent protein (GFP) coding region operatively linked to the rat insulin promoter. The insulin promoter was utilized in this construct to drive expression of GFP in pancreatic tissue.

FIG. 52. Design of the episomal mini-adenoviral vector containing FVIII cassette. The mini-Ad vector is designed to form a circularized plasmid structure that contains episomal maintenance mechanism and the FVIII expression cassette, after the viral vector enters the target cells. The general structure of the vector has the following components: (a) Recombinase expression cassette; (b) Origin of replication; (c) Human FVIII cDNA; (d) Recombinase target sites; (e) Adenovirus ITRs; and (f) Stuffer DNA sequence.

FIG. 53. General structure of the first version of the anticancer super-Ad vectors. The viral vectors consist of the Ad-helper and the super-Ad that contains multiple genes for cancer suppression and anticancer immunomodulation. The genes selected to be delivered are depicted in the diagram.

FIG. 54. General structure of the second generation of the anticancer super-Ad vectors. The viral vectors consist of the Ad-helper and the super-Ad that contains multiple genes for cancer suppression and anticancer immunomodulation. The general structure is similar to the first version of the vectors.

### DETAILED DESCRIPTION OF THE INVENTION

Within this application, unless otherwise stated, the techniques utilized may be found in any of several well-known references including: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Antibodies: A Laboratory Manual (Harlow and Lane. 1988. Cold Spring Harbor Laboratory, Cold Spring Harbor, N.Y.), Guide to Protein Purification: Methods in Enzymology, Vol. 182 (M. P. Deutscher, ed. Academic Press, San Diego, Calif.), Methods in Enzymology, Vol. 225 (see references 57-60), and Teratocarcinomas and embryonic stem cells—a practical approach (Robertson, E. J., Ed. 1987. IRL Press, Washington, D.C.; see also reference 61). Each of the references described within this application are hereby incorporated by reference.

A transcriptional regulatory region or transcriptional control region is defined as any nucleic acid element involved in regulating transcription of a gene, including but not limited to promoters, enhancers, silencers and repressors. A DNA fragment is defined as segment of a single- or double-stranded DNA derived from any source. A DNA construct is defined a plasmid, virus, autonomously replicating sequence, phage or linear segment of a single- or double-stranded DNA or RNA derived from any source.

An expression cassette is a DNA fragment comprising a coding sequence for a reporter or effector gene operably linked to a transcriptional regulatory region or a transcriptional control region sufficient for expression of the encoded protein in an appropriate cell type. A reporter construct is defined as a subchromosomal and purified DNA molecule comprising a gene encoding an assayable product. An assayable product includes any product encoded by a gene that is detectable using an assay. Furthermore, the detection and quantitation of the assayable product is anticipated to be directly proportional to the level of expression of the gene. An effector gene is defined as any gene that, upon expression of the polypeptide encoded by the gene, confers an effect on an organism, tissue or cell. A transgene is defined as a gene that has been inserted into the genome of an organism other than that normally present in the genome of the organism. Stable gene expression is defined as gene expression that may be consistently detected in a host for at least a period of time greater than seven days. A gene expressed in a tissue-specific manner is that which demonstrates a greater amount of expression in one tissue as opposed to one or more second tissues in an organism.

A recombinant adenoviral vector is defined as a adenovirus having at least one segment of heterologous DNA included in its genome. Adenoviral particle is defined as an infectious adenovirus, including both wild-type or recombinant. The adenovirus includes but is not limited to a DNA molecule encapsidated by a protein coat encoded within an adenoviral genome. A recombinant adenoviral particle is defined as an infectious adenovirus having at least one portion of its genome derived from at least one other source, including both adenoviral genetic material as well as genetic material other than adenoviral genetic material. Heterologous DNA is defined as DNA introduced into an adenoviral construct that was isolated from a source other than an adenoviral genome.

A treatable condition is defined as a condition of an organism that may be altered by administration of a form of treatment including but not limited to those treatments commonly defined as being of medicinal origin. A genetic condition is defined in this application as a condition of an organism that is a at least partially the result of expression of at least one specific gene including but not limited to the wild-type form of that gene and any mutant form of that gene.

An antigen is defined as a molecule to which an antibody binds and may further include any molecule capable of stimulating an immune response, including both activation and repression or suppression of an immune response. A tumor suppressor gene is defined as a gene that, upon expression of its protein product, serves to suppress the development of a tumor including but not limited to growth suppression or induction of cell death. A growth suppressor gene is defined as a gene that, upon expression of its protein product, serves to suppress the growth of a cell. An oncogene is defined as a cancer-causing gene. An immunomodulatory gene is defined as any gene that, upon expression of its nucleic acid or protein product, serves to alter an immune reaction. A ribozyme is defined as an RNA molecule that has the ability to degrade other nucleic acid molecules.

In at least one of its aspects, the present invention provides a modified adenoviral (Ad) vector in order to provide: 1) a large capacity Ad vector (a “mini-Ad” vector) having the capacity for insertion of up to 37 kb heterologous DNA that may also include elements for controlling transgene expression, assisting in integration of exogenous DNA into target cell genomic DNA, and/or maintenance of the vector in an episomal form within a target cell; 2) a cognate helper Ad vector designed to support propagation of the of the mini-Ad vector and that has a manipulated packaging signal such that within a host producer cell the mini-Ad vector is packaged at a greater frequency than the helper Ad vector; and, 3) a helper cell line designed to support propagation of both the mini-Ad vector and the helper Ad vector that may also serve to control transgene expression during viral propagation and selectively attenuate packaging of the helper Ad genome.

In one embodiment, the present invention comprises three components useful in generating a viral vector capable of delivering a therapeutic gene such as the FVIII cDNA to a target tissue in vivo. The components consist of a helper virus, a miniviral genome, and a helper cell line. The helper virus and helper cell line are utilized to package the miniviral genome into viral particles for gene delivery. The miniviruses generated using this system have identical tropism and host range as the adenoviral strain from which the helper virus was derived.

In other embodiments, the present invention further provides modifications of a mini-Ad vector to comprise elements derived from the adeno-associated virus (AAV). The elements are those having the ability to promote integration of genetic material into a host cell genome. In the present invention, the elements are utilized to promote integration of a reporter or effector gene of a mini-Ad vector into the host cell genome. In this manner, expression of the gene is observed in the host cell for a longer period of time than that of a conventional adenoviral vector.

The present invention further provides in certain embodiments a mini-Ad vector comprising elements for maintaining the vector as an episome in the host cell to prolong expression of the delivered gene or genes. It has been determined that limited replication of the viral genome of E1-deleted viruses in the host cell allows for longer term expression of the gene of interest as compared to those genomes that are not able to replicate (88). Deletion of the E2 region of the adenoviral genome decreases the replication and duration of gene expression from the E2-deleted adenoviral vector. It is, therefore, an objective of this invention to incorporate into the mini-Ad vector of the present invention DNA sequences derived from the normal cellular genome or equivalent sequences that will facilitate DNA replication of the mini-Ad genome in the target cell. One such sequence that facilitates DNA replication is alphoid DNA. A 16.2 kb sequence of alphoid DNA repeats allows DNA replication but not segregation of the DNA as an artificial chromosome. The present invention provides for the incorporation of the 16.2 kb sequence (70-72) into the mini-Ad vector. Replication of the mini-Ad vector containing these sequences therefore extends the persistence of the mini-Ad vector DNA and expression of the gene of interest within the target cell.

Other embodiments of the present invention include animal model test systems for evaluating the modified vectors of the present invention are also provided. Animal models provided by the present invention include: 1) a transgenic mouse comprising the AAVS1 sequence incorporated into its genome for evaluating AAV-based integration mechanisms; and, 2) a non-human transgenic animal comprising the human FVIII gene operably linked to a developmentally-regulated promoter inserted into its genome. In the second model, transient expression of human FVIII in the transgenic non-human animal during development results in tolerization of the animal to human FVIII. The human FVIII gene of the animal is not expressed once the animal matures and, therefore, evaluation of delivery of the human FVIII gene to the animals is not complicated by hFVIII expression from the transgene or by immune responses directed to hFVIII. In this manner, then, the efficiency of gene delivery by a vector may be assessed without the added potential complication provided by an anti-hFVIII immune response by the animal.

In other embodiments, Ad vectors are provided that are useful in expressing immunogenic proteins or peptides in a host, such as a human being or other animal. For instance, the Ad vectors of the present invention may provide for expression of immunogenic material related to a viral (i.e, Human Immunodeficiency Virus (HIV), Human Papilloma Virus (HPV), Herpes Simplex Virus (HSV)), bacterial (i.e., mycoplasma, streptococcus, chlamydia), fungal (i.e., Aspergillus, Candida), parasitic (i.e., Treponema, Leshmania) or other pathogen, such that the host generates an immune response against the pathogen. It is preferred that the immune response provides for resistance to infection or elimination of a current condition, such as an infection.

I. The Basic Concept of the Mini-Ad Vector System

1. Composition of the system In one embodiment, the mini-Ad vector system consists of three major parts: 1) a helper Ad that may be packaging-deficient; 2) a mini-Ad vector having a minimal amount of the viral genome; and, 3) an Ad helper cell line that provide functions of E1 trans-activation like 293 cells and/or regulation of packaging signal for the helper Ad. The helper Ad generally comprises the viral genetic material required for self-replication as well as trans-complementation of mini-Ad vector replication. The helper Ad retains wild-type Ad genetic material except for an E1 deletion or substitution and, in certain embodiments, a manipulated packaging signal for controlling or discriminating against packaging of the helper Ad in favor of packaging a mini-Ad vector of the present invention.

The mini-Ad vector typically comprises minimal Ad genetic material including only the inverted terminal repeats (ITRs) and a wild-type packaging signal as cis-elements that serve to promote replication and packaging of the mini-Ad vector. The remainder of the mini-Ad vector comprises transgene or heterologous DNA.

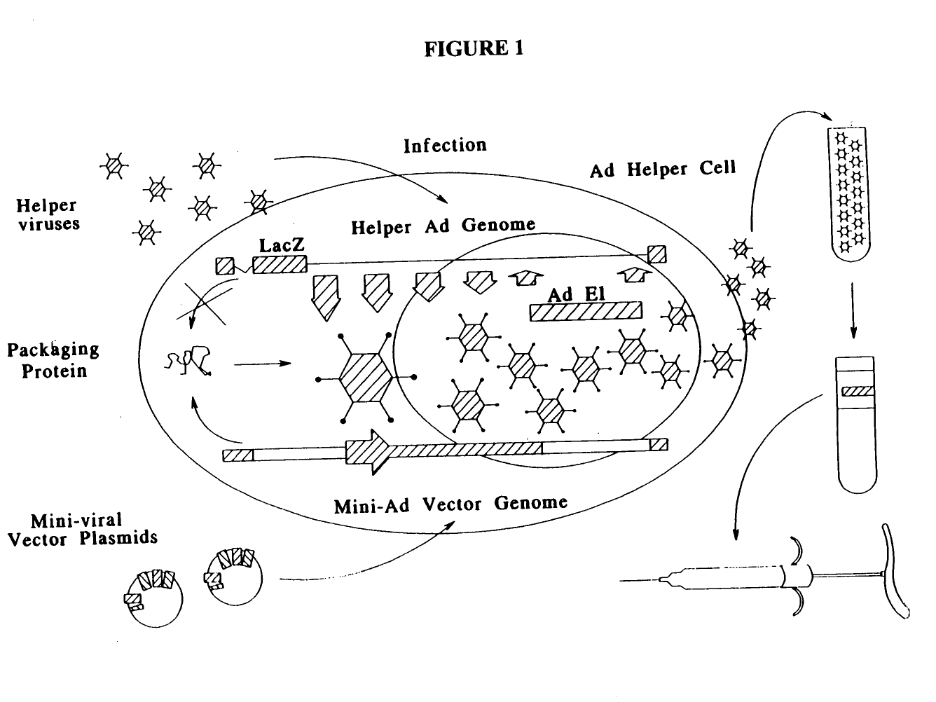

Exemplary Ad helper cell lines of the present invention are similar to 293 cells (ATCC# CRL1573) in that the cell lines comprise the Ad E1 genes and provide Ad E1 gene products that support replication of the helper Ad. The cell lines may further comprise a control mechanism for attenuating packaging of the helper Ad (FIG. 1).

2. Mechanism of operation of the system The packaging protein of Ad is a trans-acting factor present in low amounts in an infected cell and serves as the rate-limiting factor in the packaging of Ad. As the wild-type packaging signal, possessed by the mini-Ad vector of the present invention, is recognized by the packaging protein with higher affinity than the manipulated packaging signal of the helper Ad, packaging of the helper Ad genetic material is partially or completely suppressed in the presence of the mini-Ad vector. This results in preferential packaging of the mini-Ad vector. In order to replicate and package the mini-Ad vector at high-titer, the proteins for viral DNA replication and those for capsid assembly must be provided in adequate amounts. The proteins may be provided from several different sources, including but not limited to a plasmid, a cell line, or a virus. In a preferred embodiment of the present invention, the proteins are provided by the helper Ad. The present invention allows for the helper Ad to remain fully functional in replicating itself within a helper cell such that large quantities of Ad structural proteins are available to the mini-Ad vector. In the absence of the mini-Ad vector and without attenuation of the packaging signal, the helper Ad is typically packaged, albeit slowly or ineffectively.

Viral DNA replication proteins are also required to amplify the mini-Ad vector DNA for generation of multiple copies of the mini-Ad vector. The replication proteins may be provided from any of several different sources, including but not limited to a plasmid, a cell line, or a virus. In a preferred embodiment of the present invention, the proteins are provided by the helper Ad. The mini-Ad vector, comprising the wild-type packaging signal, is packaged into Ad virions as infective, replication-competent Ad particles.

In contrast, a packaging-attenuated helper Ad DNA is competed off by poor recognition or low affinity of the packaging protein for the manipulated packaging signal, and thus remains completely or partially free within the helper cells. By attenuating packaging of the helper Ad and selecting for packaging of the mini-Ad vector, this system of the present invention results in preferential propagation of the mini-Ad vector. The mini-Ad vector produced using this system may be contaminated by low amounts of helper Ad, thus the mini-Ad particle preparation may not be 100% pure. If necessary, the contaminating helper Ad may be removed using biological, biochemical, or physical methods including but not limited to ultracentrifugation through a CsCl gradient.

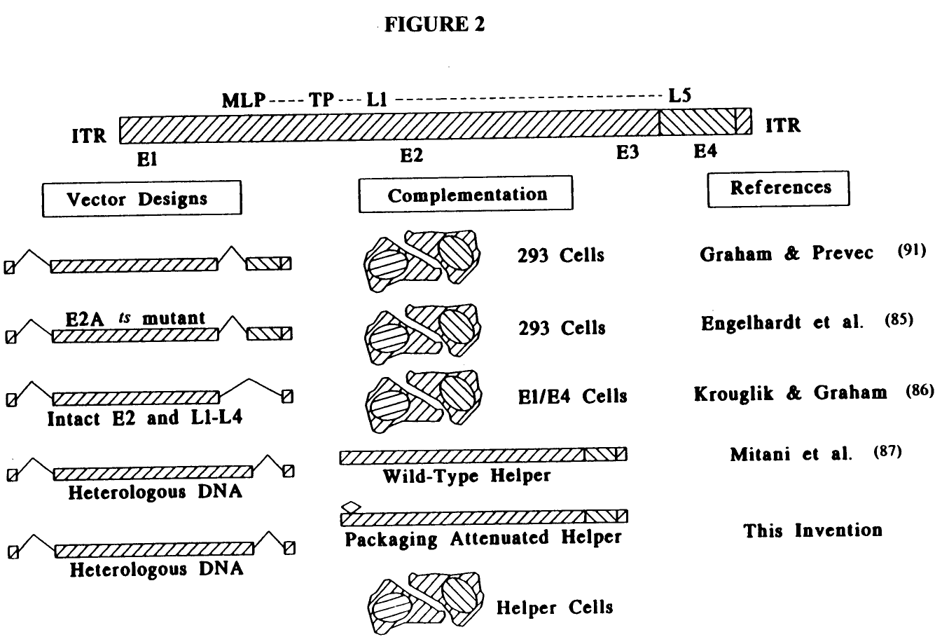

3. Capability of the system Three major features of the mini-Ad vector system of the present invention provide significant advantages over Ad vectors that are currently available to one skilled in the art FIG. 2. These features include but are not limited to the following: 1) the mini-Ad vector exhibits minimal immunogenicity to the vector itself; 2) the mini-Ad vector is virtually incapable of generating replication competent adenovirus (RCA); and, 3) the mini-Ad vector may comprise much larger segments of heterologous DNA than conventional Ad vectors. Reduced adenoviral immunogenecity and RCA generation (a major safety concern in the field of gene therapy) is possible because the mini-Ad vectors carry only a minimal amount of viral cis-element (ITRs and packaging signal), and in preferred embodiments, do not encode Ad proteins. A major source of immunogenicity and cytotoxicity of the currently available Ad vectors has thus largely been removed. The cytotoxic, inflammatory, and immunogenic responses normally resulting from expression of Ad viral proteins within a host cell or upon its cell surface are thus reduced.

The mini-Ad vector of the present invention further provides increased capacity for heterologous DNA than convention Ad vectors. Wild-type Ad has an average genome size of 36 kb. The maximal packaging capacity of Ad is roughly 105% of the genome (i.e., approximately 38 kb). The mini-Ad vector of the present invention preferably comprises less than 1 kb of Ad genetic material; therefore, the capacity of the mini-Ad vector for heterologous DNA may be 37 kb. The heterologous DNA may include but is not limited to a transgene expression cassette, a regulatory element, or a transcriptional control region operatively linked to a reporter or effector gene. The expression cassette may include but is not limited to single or multiple expression cassettes. The regulatory element may include but is not limited to a DNA sequence for controlling transgene retention, integration, transcription, and/or vector targeting.

4. The Packaging-Attenuated Helper Ad

a. The prototype structure of the helper In some embodiments, the helper Ad vector comprises a wild-type Ad genome having a manipulated packaging signal and an altered E1 gene. For safety reasons, the helper Ad must be defective in replication, such as the currently available E1-deleted or substituted viral constructs. For the purpose of controlling packaging in the presence of the mini-Ad vector, the helper must be also defective in packaging (detailed below). Therefore, the general structure of the helper can be summarized as an Ad vector having a wild-type genome except that the E1 region and packaging signal are manipulated. However, other essential regulatory genes such as E2 and E4, for example, may also be manipulated. The viral genome may be split into fragments in order to further disable the replication competence of the helper Ad or to reduce the genome size of the helper Ad in order to separate it from the mini-Ad vector using a biological, biochemical, or physical method including but not limited to ultracentrifugation through a CsCl gradient. As long as the titer of the helper Ad is not significantly affected, both a defect in viral replication and attenuation in packaging of the helper Ad may be included in the design of the helper Ad.

b. The general function of the helper Ad The primary function of the helper Ad is to supply the capsid proteins required to package the mini-Ad vector. In order to provide the proteins, the helper Ad must be able to replicate within the host cell, although less efficiently than wild-type Ad. Preferably, DNA replication and transcription of the helper genome is not affected. If synthesis of the helper Ad genome were inhibited, the yield of the late gene products (the capsid proteins) would be altered and may adversely affect the titer of the mini-Ad vector (i.e., the titer will be reduced). For certain applications, removal of the helper Ad from the mini-Ad may not be necessary. In such situations, the stringency of packaging attenuation of the helper Ad may be greatly reduced.

c. Designs for packaging attenuation The purpose for attenuation of packaging the helper Ad is to reduce the potential for helper Ad contamination in preparations of the mini-Ad vector. This is especially important when a relatively pure batch of the mini-Ad vector is required for a particular application. The packaging function of the helper Ad is designed to be defective but not completely disabled, because the helper Ad must be able to propagate, albeit slowly, in the absence of a mini-Ad vector. The following genetic manipulations may be utilized to generate a packaging-attenuated helper Ad.

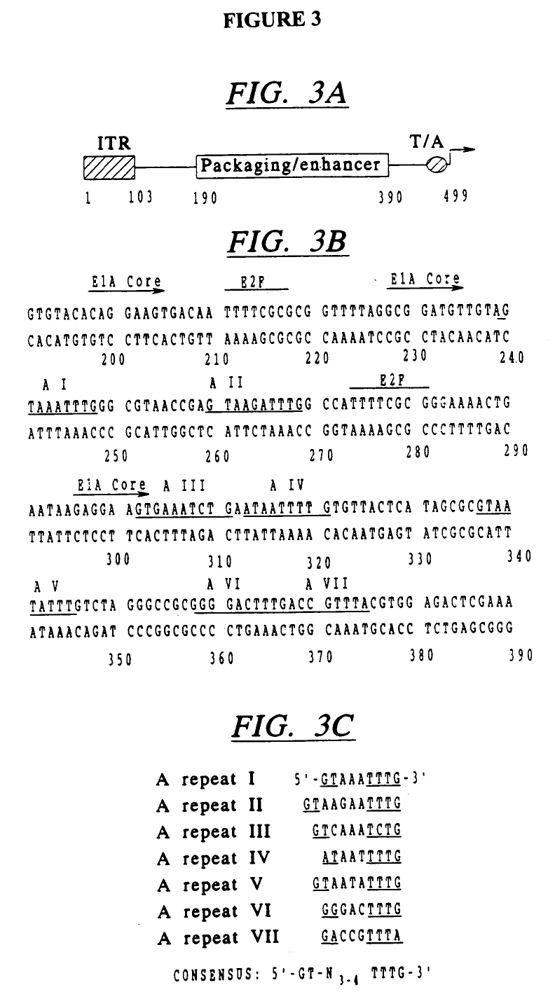

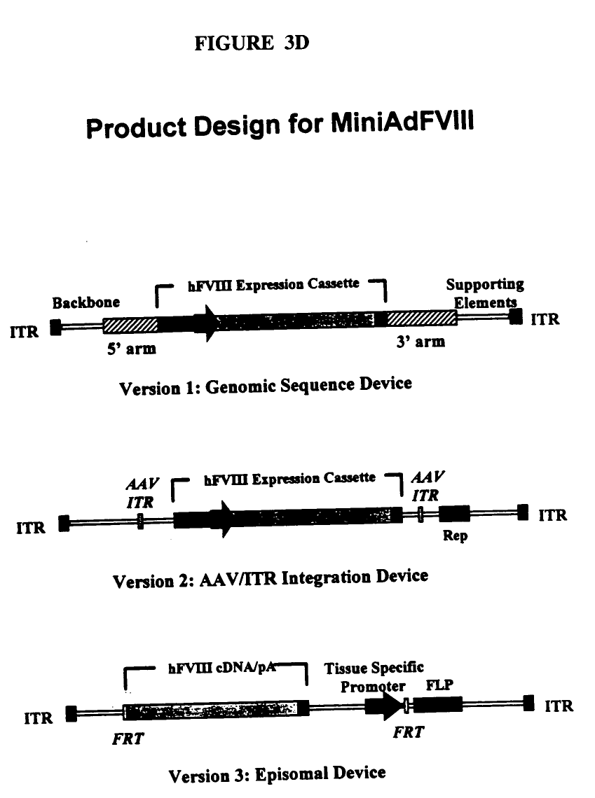

1. Packaging signal mutation The Ad5 packaging signal is composed of a repeated element that is functionally redundant (18). Partial deletions of the packaging signal elements have been shown to reduce the yield of mutant Ad from several fold to approximately a hundred fold as compared to that of Ad having a wild-type packaging signal (18). The design of the packaging signal mutation of the present invention may therefore incorporate a partial deletion of the consensus adenosine-enriched motif (e.g. “A-repeat”: TAAATTTG; FIG. 3) from the wild-type Ad packaging signal.

2. Synthetic packaging signal Since the Ad5 packaging signal has a consensus A (adenosine) enriched motif (e.g. A-repeat: TAAATTTG), incorporation of an array of tandem repeats including but not limited to a selected A-repeat or any synthetic DNA motifs that may alter the affinity of the packaging protein for the artificial packaging signal.

3. Packaging signal interference The Ad packaging signal is a specific DNA sequence that is recognized and bound by the packaging proteins. In order to interfere with the effective binding of the packaging proteins to the signal, other DNA sequences may be placed in proximity to or within the A-repeat array of the helper Ad packaging signal. The inserted DNA sequences allow binding by their cognate DNA binding proteins that may positionally compete off the binding of the Ad packaging proteins to the Ad packaging signal.

4. Packaging signal relocation The wild-type Ad packaging signal is positioned at the left end of the wild-type Ad genome. Investigators have found that the packaging signal may be located at the right end and retain its function (75) indicating that the packaging signal may be relocated. Positioning the manipulated packaging signal in a location other than wild-type may be useful to further attenuate the packaging efficiency of the helper Ad. In addition, relocation of the packaging signal to another region of the Ad genome may be helpful in minimizing the possibility of reversion of the helper Ad back to wild-type Ad through homologous recombination between the engineered packaging signal of the helper Ad and the wild-type packaging signal of the mini-Ad vectors (i.e., generation of RCA).

5. Further possibilities To attenuate packaging of the helper Ad to minimize the contamination of the helper to a preparation of the mini-Ad vectors, two factors may be considered: cis-elements and trans-acting factors. Therefore, other possible designs may be oriented towards manipulation of either or both of these two factors. An example of cis-elements that may be manipulated is the A-repeat motif. An example of a trans-acting factor that may be manipulated is a packaging protein. Further consideration should be a controllable mechanism of packaging without sacrificing the high titer output of the mini-Ad vectors by the system.

II. The Mini-Ad Vector

A. The basic structure of the mini-Ad vector Ad vectors may be utilized as circularized plasmids by fusion of the Ad ITRs (54). The simplest plasmid form of the mini-Ad vector of the present invention is a circular DNA molecule comprising an ITR fusion sequence (comprising an Ad ITR having a wild-type packaging signal), a plasmid DNA replication origin, and a polycloning site consisting of one or multiple restriction enzyme sites. The ITR fusion sequence includes the left end of the wild-type Ad, preferably from map unit 0 to 1, and the right end, preferably from map unit 99 to 100. An Ad DNA replication origin is located in each ITR and the wild-type packaging signal is located adjacent to the left ITR.

B. The structural and functional possibilities of the mini-Ad vectors Other DNA sequences and elements including but not limited to those listed below may be included in a mini-Ad vector.

1. Expression cassettes of transgenes An expression cassette is a basic transcription unit. A simple expression cassette of a given gene generally comprises a transcriptional control region, a gene of interest (i.e., heterologous DNA, insert DNA), and a polyadenylation (polyA) signal. Within an expression cassette, two or more genes may be included as bi- or polycistronic units, as long as additional elements for translation or splicing of RNA are provided between the genes. Generally, mini-Ad vectors comprise one or multiple expression cassettes.

2. Functional elements for vector DNA retention Elements that may assist in integration of the expression cassette into target cell genome (i.e., AAV integration elements) or maintain the mini-Ad vector as an episomal form in a host cell (FIG. 3B). Elements that have been shown to assist in integration are the inverted terminal repeats (ITRs) and the Rep78/68 proteins of the adeno-associated virus (AAV). AAV utilizes these elements to achieve specific integration of its genome in human chromosome 19 (19q13.3-qter) at a site named AAVS1.

Although AAV has been considered as a candidate vector for gene therapy, several limitations have been identified by investigators. AAV is limited by: 1) low capacity for exogenous DNA (4.3 kb); 2) difficulty in achieving high titers in large-scale preparations; and, 3) loss of specific integration of the recombinant AAV. Each of these have proven to be difficult challenges to those skilled in the art. The present invention combines the advantages of the mini-Ad vector with the integration capacity of AAV by incorporating the AAV-ITR sequences and Rep 78/68 expression cassette (Rep expression cassette) into the vector.

Mechanisms that may also be included in the mini-Ad genome include extrachromosomal replication sequences (70). Such sequences, comprised of either chromosomal or viral sequences, serve to enable the vector to efficiently replicate and be retained within a mammalian cell. The sequences may include a replication component such as human genomic DNA and/or a retention component such as human centromere sequence or sequence derived from the Epstein-Barr virus (EBV) such as the oriP family of repeats and/or EBNA-1 (70). The human human genomic DNA may comprise a telomere and/or alphoid DNA (70). By including such elements in a mini-Ad vector, the mini-Ad genome will replicate to a higher copy number in the host cell, thus increasing the probability that the mini-Ad genome will be packaged at a greater effiency than that helper virus. Additionally, these sequences serve to lengthen the duration of expression of the effector or reporter gene within the host cell. Such functions would be useful in utilization of the mini-Ad vector for gene therapy.

3. Regulatory elements for control of DNA transcription Elements having transcriptional regulatory function including but not limited to enhancers, repressors, activator-binding sites, introns, and 5′ or 3′-untranslated regions. Various combinations of such elements may be incorporated into the mini-Ad to enhance or control expression of a gene of interest.

4. Elements for vector and transgene targeting Targeting can be achieved by several methods including but not limited to vector surface modification and tissue-specific expression. Tissue specific promoters may be utilized to drive gene expression in a specific cell type or tissue. Many such promoters are available to one of skill in the art.

5. Further supporting elements These may include but are not limited to DNA replication origins for prokaryotic or eukaryotic cells, plasmid or vector selection markers, and vectors backbones. The skilled artisan would understand the need to incorporate one or more of such supporting elements into the mini-Ad vector as necessary.

C. Designs for high titer production of the mini-Ad vectors High-titer production of the mini-Ad vectors is another major aspect of this invention. One advantage of Ad vectors over other viral vectors is that Ad particles are conducive to preparation of high-titer preparation stocks (67). High-titer propagation of Ad is possible due mainly to the large quantity of viral capsid proteins and viral genome copies produced wtihin a host cell such as a 293 cell during infection. Listed below are some of the factors that may be considered in designing methods for generating high-titer mini-Ad vectors.

1. Enhanced DNA replication Ad has its own enzymatic system for DNA replication. The E2 region proteins are the major trans-acting elements responsible for viral DNA replication. The replication origins are the cis-elements located at the either or both ends of the viral genome. To support mini-Ad genome replication, a sufficient quantity of E2 proteins must be provided by the helper virus. High-level expression of E2 proteins (encoded within the E2 region of Ad) is ensured by proper design of the helper virus genome. Other such mechanisms for increase in copy numbers of the mini-Ad genome may also be considered. Such mechanisms may include but are not limited to insertion of the the SV40 origin of DNA replication (54) into the mini-Ad genome to increase the copy numbers of the mini-Ad, concomitant with SV40 T-Ag expression in the helper cell.

2. Enhanced packaging signal A higher number or more efficient packaging sequences may be utilized by, for example, incorporating a greater number of tandem repeats at one or both ends of the mini-Ad genome, or by incorporation of one or multiple synthetic packaging signals that function in a more efficient manner than the wild-type packaging signal.

3. Enhanced packaging process The packaging process and mechanism of Ad are not yet fully understood by those skilled in the art. Whether DNA binding proteins other than the packaging signal of Ad have synergistic roles for packaging is not certain. The sequences for which a DNA-binding protein shows affinity, referred to as “anchorage points for packaging” and naturally existing within the Ad genome, may be incorporated into the mini-Ad vector.

D. The Ad Helper Cell Lines

1. The basic elements and general function of the Ad helper cells The cell line of the present invention (that serves as the host cell) provides several important modifications that improve upon the conventionally utilized cell line, 293 (ATCC# CRL1573). In a preferred embodiment, the host cell comprises a nucleic acid sequence encoding an Ad-E1 fragment for trans-activation of the transcription program of the helper Ad genome FIG. 1. Unique from the E1 fragment of 293 cells currently available to one skilled in the art, a cell line of present invention may comprise nucleic acid sequence encoding the E1 fragment having no overlapping nucleic acid sequence with the helper Ad genome. The present invention, therefore, eliminates one of the current difficulties associated with Ad vectors: generation of wild-type Ad or replication-competent Ad (RCA) through homologous recombination. Other elements may include but are not limited to genes involved in the support of high copy-number production of the mini-Ad vector, enhancing packaging of the mini-Ad vector, and/or attenuating the packaging of the helper Ad.

2. Assistance mechanisms for packaging attenuation of the helper Ad Other methods by which packaging of the helper Ad is attenuated may include interference with the binding site for the packaging protein by placement of a binding site for a different protein nearby the packaging protein binding site within the helper Ad genome. Such a system may include but is not limited to utilization of the tetracycline-repressor (Tet-R), a recombinase, and/or an altered packaging protein. In a preferred embodiment, the different protein is expressed within a host cell. Tet-R may bind to a manipulated packaging signal of a helper virus comprising a binding site for Tet-R the tet-operon (Tet-O), and thereby repress packaging by inhibiting binding of the packaging protein. Binding of Tet-R to Tet-O is controlled by tetracycline. Addition of tetracycline into the cell culture medium results in binding of tetracycline to the Tet-R and prevents it from binding tet-O. Removal of the tetracycline frees Tet-R for binding to the engineered packaging signal and serves to further attenuate packaging of the helper virus.

Expression of a recombinase such as Cre or Flp may also inhibit packaging provided the packaging signal of the helper virus is flanked by a recombination site, such as lox-p or FRP (FIG. 3B), respectively (66, 68). Other genetic modifications within the helper virus genome may also be provided separately or in addition to those listed above to further attenuate helper virus replication.

The packaging protein may be altered by any of several methods including but not limited to utilization of a specific serotype or species difference in the packaging signal to differentiate packaging of the mini-Ad from the helper Ad provided the specific packaging protein of Ad is identified. Additionally, the packaging protein may be altered by genetic modification of the gene encoding the packaging protein. The modification may alter the packaging protein such that its binding preference for the wild-type packaging signal is increased. The modified packaging protein, then, may further provide preferred packaging of the mini-Ad genome.

3. Assistance mechanisms for high-titer production of the mini-Ad vectors Modifications of the mini-Ad vector designed to increase the copy number of the mini-Ad genome within a host cell are useful in the development of a high-titer mini-Ad vectors. Expression of the SV40 T-Ag (mutated T-Ag with no transforming activity) by the host cell may increase the copy number of the mini-Ad genome, provided a SV40 DNA replication origin is incorporated into the mini-Ad plasmid vector.

IV. Potential Applications of the Present Invention

a. Delivery of genes for therapy of genetic diseases in vivo Large capacity for exogenous nucleic acid is necessary for delivery of a large therapeutic gene or multiple genes as well as for transfer of regulatory elements and/or other related genes along with the primary therapeutic genes that will determine controllable or tissue-specific expression and may result in a more effective therapeutic effect. An example includes but is not limited to cystic fibrosis in which controllable expression of several genes is required to optimize cystic fibrosis gene therapy. Gene therapy of Duchenne muscular dystrophy (DMD) is another example of a condition for which treatment would require a large capacity vector. For treatment of this disease, genes including but not limited to muscle and nerve growth factors may be required to be co-delivered in order to generate a complete physiological effect to restore the muscle function of the patients.

b. Induction of host anti-cancer immunity through intratumoral injection of the vectors Ad vectors demonstrate high levels of infectivity in cultured tumor cells and different types of solid tumor models in vivo. This characteristic of the Ad vector has been utilized in the treatment of cancer. The efficacy of treatment depends upon the genes that are delivered by the vectors. Multiple genes including but not limited to those having combined functions of tumor suppression and immunomodulation are utilized to optimize the anti-cancer effect. The mini-Ad vector has the capacity to deliver multiple genes and is useful in constructing anti-cancer Ad vectors for intratumoral injection.

c. Modulation of host immunity by genetic modification of the graft cells or tissues. Transplantation requires transient or permanent suppression of the host immunity. To deliver immune suppression genes into cells or tissues including but not limited to graft cells or graft tissues may be an alternative approach to the administration of immunosuppressive agents. Examples of genes encoding immune suppression proteins to be utilized in the present invention may include but are not limited to TGF-β, IL-10, viral proteins HSV-ICP47 and CMV-US11, and secretable Fas-ligand proteins that may be delivered alone or in combination by the mini-Ad vectors of the present invention.

d. Modification of target cell function or regulation target cell growth in vivo by genetic modification. Ad vectors have a distinct advantage over other viral vectors in that production of high titer stocks is possible, which is useful for in vivo gene therapy. Because the mini-Ad vectors contain only minimal amounts of cis-elements of the Ad genome, the immunogenicity of mini-Ad is minimized. Therefore, the mini-Ad vector will be useful for modifying target cell function or regulating target cell growth in vivo by genetic modification.

e. Specific delivery of transgenes to target cells or tissues in vivo by surface modification of the vectors The genes encoding the adenoviral hexon and fiber proteins are engineered to fuse with certain epitopes or ligands (e.g. the protein A that binds to Fc fragment of IgG) present on the target cell surface. These modified genes are incorporated into the recombinant viral genome for generation of the viruses having surface sites that interact with ligands that function as targeting agents on the target cell surface. The viral particles thus produced have tissue or cell recognition capabilities.

f. To be used for Ad-mediated vaccination via direct in vivo approaches For the purpose of vaccination, the immunogenicity of the E1-substituted Ad vectors may provide benefits, and has been used in development of Ad-based recombinant vaccines. Mini-Ad vectors utilized in this type of application use the helper virus including but not limited to E1-substituted Ad vectors as well as co-delivery of genes encoding antigens and immunogens that provide immunization.

g. To be used for ex vivo gene delivery Transient gene retention and expression associated with the use of conventional Ad vectors has prevented Ad from being widely used in ex vivo gene delivery protocols. The mini-Ad vectors, having DNA retention mechanisms, are useful for this purpose. Also, the high infectivity of Ad in cultured cell lines make the mini-Ad vectors very effective gene delivery system for ex vivo approaches toward gene therapy.

h. To be used as tools for basic research and development of adenovirology and novel vector construction The mini-Ad vector system itself has a great value for basic adenovirology studies. The construction and demonstration of the feasibility and operation are already a breakthrough in the field. The helper Ad and the mini-Ad provide convenient tools for study of the Ad and its potential applications. This is particularly true for the mini-Ad vector. The characterization of the replication, packaging, and propagation efficiency of the mini-Ad will provide the field with important new information, which was previously unavailable.

i. To be used in combination with other methodology in the field of gene transfer and therapy Ad vectors have been used together with polylysine, liposome, and other conjugation materials as a gene delivery complex. The mini-Ad vectors can also be used with these compounds as well as any other compound that comprise the ability to serve as a gene delivery complex.

j. To be used for other purposes in the field of gene transfer and therapy The mini-Ad vector system has a great potential to be used for gene transfer and therapy in addition to what have been discussed above. The possibilities will come across along the further development of the field of gene transfer and therapy.

k. Pharmaceutical Compositions

The vectors of the present invention may be administered by any suitable route including but not limited to oral, parenteral, inhalation, rectal, topical, intravenous (i.e., the portal vein), intrarterial (i.e., hepatic artery), intrapleural, nasal, intrathecaly, or direct intaorgan injection in dosage unit formulations containing conventional pharmaceutically acceptable carriers, adjuvants, and vehicles. The term parenteral as used herein includes but is not limited to subcutaneous, intravenous, intradermal, intramuscular, intrastemal, infusion techniques or intraperitoneally, among others. Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug. Other suitable routes would be understood by one of skill in the art.

The dosage regimen for treating a disorder or a disease with the vectors of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The pharmaceutically active compounds (i.e., vectors) of this invention can be processed in accordance with conventional methods of pharmacy to produce medicinal agents for administration to patients, including humans and other mammals. For oral administration, the pharmaceutical composition may be in the form of, for example, a capsule, a tablet, a suspension, or liquid. The pharmaceutical composition is preferably made in the form of a dosage unit containing a given amount of DNA or viral vector particles (collectively referred to as “vector”). For example, these may contain an amount of vector from about 10³-10¹⁵ viral particles, preferably from about 10⁶-10¹² viral particles. A suitable daily dose for a human or other mammal may vary widely depending on the condition of the patient and other factors, but, once again, can be determined using routine methods. The vector may also be administered by injection as a composition with suitable carriers including saline, dextrose, or water.

While the nucleic acids and/or vectors of the invention can be administered as the sole active pharmaceutical agent, they can also be used in combination with one or more vectors of the invention or other agents. When administered as a combination, the therapeutic agents can be formulated as separate compositions that are given at the same time or different times, or the therapeutic agents can be given as a single composition.

IV. Mini-Ad Vector for Treatment of Liver-Related Disease

Following intravenous injection of adenoviruses, greater than 90% of the viral particles are localized in the liver. It is in the liver that expression of adenoviral genes or a transgene of an adenoviral vector is observed following administration of the viral particle. The present invention includes a methodology for directing gene expression to the liver using a transcriptional regulatory region, or promoter, capable of driving expression of a reporter or effector gene in liver tissue in combination with a modified and much improved adenoviral vector. One skilled in the art can envision a multitude of diseases caused by abnormal gene or gene product expression of a gene in the liver. Abnormal gene or gene product expression may include a level of expression above or below that normally found in the liver and may be the result of a gene deletion, duplication, insertion, or alteration of the structure or function of the gene trasncript, or other alteration of either the gene itself or its protein product. Abnormal gene or gene product expression may also result from alteration of the transcriptional or translational machinery regulating expression of the gene and gene product, respectively.

A vector for delivery of a therapeutic or reporter gene to the liver comprising the significant advantages of the present invention. It will be understood by those skilled in the art that the present invention could be utilized to treat a multitude of diseases based on a defect in either gene or protein expression in the liver. Examples of diseases and genes that may be treated or utilized, respectively, using the present invention are summarized in Table 1. Additionally, the promoter of any of these genes may prove useful in driving gene expression in the liver for the purpose of driving expression of a gene or gene product in the liver.

TABLE 1*

| Disease | Frequency | Defect protein/gene | Malfunction manifestation | Current treatment |
|---|---|---|---|---|
| Apo A-1 structural | 1:1,000 | Apo A-1 | In a few cases, causes decreased | Dietary treatment; HMG-CoA |
| mutations | | | HDL-C levels with no increase in | reductase inhibitors |
| | | | coronary heart disease. Two | |
| | | | mutations lead to amyloidosis | |
| Familial ligand-defective | ˜1:500-1,000 | Apo B-100 | Impariment of receptor-dependent | Dietary treatment; HMG-CoA |
| apo B-100 | heterozygous | | endocytosis of LDL in all nucleated | reductase inhibitors. |
| | | | cells Elevated levels of LDL in | |
| | | | plasma. Increased risk of | |
| | | | atherosclerotic heart disease. | |
| Familial | ˜1:3,000 | Apolipoprotein B-100 | Failure to secrete VLDL and | For homozygous; Restriction of |
| hypobetalipoproteinemia | heterozygous | | chylomicrons if truncations involve | dietary fat and supplementation |
| | | | more than two-thirds of apo B-100 | of Vitamin E |
| | | | sequence. Less severe truncations | For heterozygous; Moderate |
| | | | do not prevent secretion but result | doses (400-800 mg/day) of |
| | | | in abnormal plasma lipoproteins | tocopherol |
| | | | Heterozygotes show lipid | |
| | | | abnormalities but are usually | |
| | | | asymptomatic. Homozygotes may | |
| | | | have a syndrome similar to | |
| | | | homozygous abetalipoprotinemia. | |
| Familial type III | 1:1,000-5,000 | Apolipoprotein E | Accumulation in plasma of | Dietary treatment; HMG-CoA |
| hyperlipoproteinemia | | | chylomicron and VLDL remnants | reductase inhibitors; nicotinic |
| (dysbetalipoproteinemia) | | | (collectively, β-VLDL), leading to | acid plus fibric acid derivatives. |
| | | | hyperlipidemia and atherosclerosis. | |
| Familial | 1:500 in most | LDL receptor | Absent or deficient receptor- | Dietary treatment; HMG-CoA |
| hypercholesterolemia | populations | | mediated endocytosis of LDL | reductase inhibitors; nicotinic |
| | | | causes LDL to accumulate in | acid plus bile acid-binding |
| | | | plasma. Hyper-cholesterolemia and | resins. For homozygous, may |
| | | | atherosclerosis result | need probucol, portacaval |
| | | | | anastomosis, plasma exchange, |
| | | | | and liver transplantation |
| von Willebrand disease | 1:8,000 | von Willebrand factor | Abnormal platelet adhesionand | Vasopressin analogue DDAVP |
| | | | mildly to moderately reduced factor | for mild deficiency. Plasma |
| | | | VIII levels cause bleeding | infusion and vWF treatment for |
| | | | | severe patients. |
| Factor VIII deficiency | 1:10,000 males | Factor VIII | Factor VIII fails to functionas a | Prophylaxis, plasma infusion, |
| (hemophilia A) | | | cofactor for activation of factor X | and Factor VIII treatment |
| | | | and impairs clotting cascade. | |
| Factor IX deficiency | 1:70,000 | Faxtor IX | Impaired blood coagulation. | Prophylaxis, plasma infusion, |
| (hemophilia B) | | | | and Factor IX treatment. |
| Factor XI deficiency | ˜1:1,000 in | Faxtor XI | Deficiency of protein leads to | Prophylaxis, plasma infusion, |
| (hemophilia C) | Ashkenazi Jews of | | impaired contact activation and | and Factor XI treatment. |
| | Israel | | mild bleeding tendency. | |
| Antithrombin deficiency | ˜1:5,000 | Antithrombin | Impaired inhibition of coagulation | Long-term anticoagulation |
| | | | factors IIa, IXa, and Xa in plasma | therapy, plasma or antithrombin |
| | | | causes recurrent venous thrombosis. | infusion. |
| Protein C deficiency | 1:10,000 | protein C | Impaired regulation of blood | Long-term anticoagulation |
| | | | coagulation. Predispositionto | therapy, plasma or protein C |
| | | | thrombosis | infusion |
| α₁-Antitrypsin deficiency | 1:7,000 northern | α₁-Antitrypsin | Liver storage of polypeptide, | α₁-Antitrypsin replacement |
| (Z variant) | Europeans 1:3,000 | | plasma deficiency of protein | therapy. |
| | Scandinavians | | allows overactivity of elastase | |
| C2 deficiency | ˜1:10,000 | Complement component 2 | Markedly reduced activation of the | Replacement therapy |
| | | | classic pathway | |
| Phenylketonuria (PKU) | ˜1:10,000 births | Phenylalanine hydroxylase | Hepatic enzyme deficiency causes | Low-phenylalanine-diet therapy |
| due to PAH deficiency | (considerable | (PAH) | hyperphenylalaninemia, plasma | and enzyme (bacterail |
| | regional variation) | | values persistently above 1 mM | phenlalanine ammonia lyase) |
| | | | associated with impaired cognitive | treatment. |
| | | | development. Risk of maternal | |
| | | | hyperphenyl-alaninemia effect on | |
| | | | fetus carried by female proband. | |
| Transferase deficiency | 1:35,000-60,000 | Galactose 1-phosphate | Accumulation of galactose, | Elimination of dietary lactose, |
| galactosemia | | uridyltransferase | galactitol, galactose 1-phosphate, | avoidance of galactose. |
| | | | and galactonate causes cataracts, | |
| | | | mental retardation, and liver and | |
| | | | kidney dysfunction. | |
| Hereditary fructose | 1:20,000/ | Fructose 1,6-bisphosphate | Ingestion of fructose causes the | Elimination from the diet of all |
| intolerance | Switzerland | aldolase B | accumulation of fructose 1- | sources of sucrose and fructose, |
| | | | phosphate and hence multiple | with supplement of vitamin C. |
| | | | dysfunctions in small intesting, | |
| | | | liver, and kidney | |
| Glycogen storage disease | ˜1:100,000 | Glucose 6-phosphatase | Hypoglycemia, hyperlipidemia, | Dietary restriction; nocturnal |
| type Ia (von Gierke | | | hyperuricemia, and hyperlactic | nasogastric infusion in early |
| disease) | | | acidemia. Glycogen accumulation | infancy, portacaval shunts, liver |
| | | | in liver and kidney. | transplantation |
| Glycogen storage disease | ˜1:125,000 | Amylo-1,6-glucosidase | A glycogen with shorter outer | Dietary restriction |
| type III | | (debrancher enzyme) | chains (limit dextrin) in liver and/or | |
| | | | muscle. Moderate hypoglycemia | |
| | | | and hyperlipidemia. Muscle | |
| | | | weakness mostly in adults. | |
| Menkes disease | 1:250,000 | P-type ATPase (Cu²⁺) | Defective intracellular transport of | No specific or effective |
| | | transport | copper leads to deficiency of | treatment. Presymptomatic |
| | | | copper-containing enzymes and | treatment with copper |
| | | | causes arterial and brain | histidinate can modify the |
| | | | degeneration | disease substantially |
| Wilson disease | 1:50,000 | P-type ATPase | Defective biliary excretion of | Penicillamine, trientene or |
| | | [membrane cation (Cu²⁺) | copper leads to accumulation in | orraly administered zinc salts |
| | | transporter] | lever (cirrhosis), cornea (Kayser- | Liver transplantation may be |
| | | | Fleischer rings), and basal ganglia | applied for irreversible liver |
| | | | (movement disorder). | damage. |
| Carbamyl phosphate | 1:70,000-100,000 | Carbamyl phosphate | Impaired urea formation leads to | Dietary restriction, sodium |
| synthetase deficiency | | synthetase I | ammonia intoxication | phenylbutyrate, and citrulline. |
| Ornithine | 1:70,000-100,000 | Ornithine | Impaired urea formationleads to | Dietary restriction, sodium |
| transcarbamylase | | transcarbamylase | ammonia intoxication | phenylbutyrate, and arginine. |
| deficiency | | | | |
| Argininosuccinic acid | 1:70,000-100,000 | Argininosuccinic acid | Impaired urea formation leads | Dietary restriction and arginine |
| synthetase deficiency | | synthetase | tooammonia intoxication | |
| Methylmalonic acidemia | 1:20,000 | Methyldmalonyl-CoA | Accumulation of methylmalonate | Dietary protein restriction and |
| (2 allelic variants | | mutase (MUT) apoenzyme | leads to metabolic ketoacidosis and | oral antibiotic therapy |
| designated mut° and mut-) | | | developmental retardation | |
| Methylmalonic acidemia | 1:20,000 | cblA form unknown cblB | Impaired adenosylcobalamin | Cyanocobalamin or |
| (2 nonallelic forms | | form ATP cob(I)alamin | syntheses leads to deficient | hydroxocobalamin treatment |
| designated cblA and | | adenosyltranferase | methylmalonyl-CoA mutase (MUT) | with dietary protein restriction |
| cblB) | | | activity; clinical and chemical | |
| | | | findings resemble those in | |
| | | | apoprotein MUT deficiency | |
| Xanthinuria | 1:45,000 | Xanthine dehydrogenase | Accumulated substrate (xanthine) | No specific or effective therapy |
| | | (xanthine oxidase) | can crystallize inkidney, urinary | is available. Avoidance of |
| | | | tract, or muscle, causing renal | purine-rich foods is advised. |
| | | | failure, nephrolithiases, or | |
| | | | myopathy. Impaired/distrubed drug | |
| | | | metabolism. | |

*Table 1 is composed of the data and information from Ref. No. 55.

Yet another embodiment of the present invention includes a pharmaceutical composition comprising a mini-Ad vector of the present invention. The pharmaceutical compositions may be made up in a solid form (including granules, powders or suppositories) or in a liquid form (e.g., solutions, suspensions, or emulsions). Solid dosage forms for oral administration may include capsules, tablets, pills, powders, and granules. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting sweetening, flavoring, and perfuming agents. The compounds of the present invention can be used in the form of salts derived from inorganic or organic acids.

While the vectors of the invention can be administered as the sole active pharmaceutical composition, they can also be used in combination with one or more vectors of the invention or other agents. When administered as a combination, the therapeutic agents can be formulated as separate compositions that are given at the same time or different times, or the therapeutic agents can be given as a single composition.

IV. A Mini-Ad Vector for Replacement of FVIII Activity

An E1-substituted adenoviral vector comprising the FVIII gene has been utilized to achieve therapeutic levels of FVIII expression in the host animal (13). Multiple difficulties have been encountered, however, by investigators attempting to utilize adenoviral vectors in gene therapy. The present invention provides a solution for many of these problems, as described below.

As the essential cis-acting elements for Ad DNA replication and packaging are located at the ends of the viral genome (ITRs plus the packaging signal, less than 1 kb), the backbone of the mini-Ad vector has been modified to comprise only the essential cis-elements. The remainder of the mini-Ad genome, up to its packaging limit of approximately 38 kb, may be comprised of heterologous DNA. In the present invention, the heterologous DNA comprises a nucleic acid sequence encoding a protein having activity similar to that of human FVIII. FVIII is normally produced in the liver and is comprised of heavy chain polypeptides with a range of apparent molecular weights of from 92 kDa to 210 kDa derived from the amino terminus of the nascent polypeptide and a C-terminal light chain of 80 kDa (53). The activated form functions in the blood clotting cascade as a cofactor along with activated factor IX (FIXa), negatively charged phospholipids and calcium ions to convert factor X to its activated form, Xa. The human cDNA is 9 kb in length and encodes a polypeptide of 2351 amino acids comprised of several domains in the order A1, A2, B, A3, C1 and C2 (5-7). The A and C domains are critical for functional activity whereas the majority of the B domain, consisting of approximately 980 amino acids, is dispensable for activity (8). The present invention, as an example of a mini-Ad vector having FVIII-like activity, provides a mini-Ad vector comprising the human FVIII gene. Various naturally occurring and recombinant forms of Factor VIII have been described in patent and scientific literature such as U.S. Pat. No. 5,563,045, U.S. Pat. No. 5,451,521, U.S. Pat. No. 5,422,260, U.S. Pat. No. 5,004,803, U.S. Pat. No. 4,757,006, U.S. Pat. No. 5,661,008, U.S. Pat. No. 5,789,203, U.S. Pat. No. 5,681,746, U.S. Pat. No. 5,595,886, U.S. Pat. No. 5,045,455, U.S. Pat. No. 5,668,108, U.S. Pat. No. 5,633,150, U.S. Pat. No. 5,693,499, U.S. Pat. No. 5,587,310, U.S. Pat. No. 5,171,844, U.S. Pat. No. 5,149,637, U.S. Pat. No. 5,112,950, U.S. Pat. No. 4,886,876, WO 94/11503, WO 87/07144, WO 92/16557, WO 91/09122, WO 97/03195, WO 96/21035, WO 91/07490, EP 0 672 138, EP 0 270 618, EP 0 182 448, EP 0 162 067, EP 0 786 474, EP 0 533 862, EP 0 506 757, EP 0 874 057, EP 0 795 021, EP 0 670 332, EP 0 500 734, EP 0 232 112, EP 0 160 457, Sanberg et al., XXth Int. Congress of the World Fed. Of Hemophilia (1992), and Lind et al., Eur. J. Biochem., 232:19 (1995).

Nucleic acid sequences encoding Factor VIII may be prepared by recombinant methods, such as by screening cDNA and/or genomic DNA libraries from cells expressing Factor VIII or by deriving the sequence from a vector known to include the same. The desired sequence may also be isolated directly from cells and tissues expressing FVIII mRNA, using standard techniques, such as PCR of cDNA and/or genomic DNA (See e.g., Sambrook et al., supra, for a description of techniques used to obtain and isolate DNA). Such nucleotide sequences may also be produced synthetically. For instance, the sequence may be assembled from overlapping oligonucleotides and assembly of the oligos into a complete coding sequence (i.e., Edge, Nature 292:756 (1981); Nambair et al., Science 223:1299 (1984); and Jay et al., J. Biol. Chem., 259:6311 (1984)). It should be understood by those skilled in the art that a mini-Ad vector comprising genetic material that encodes a protein having FVIII-like activity (i.e., the ability to function as a cofactor in the conversion of factor X to its activated form, Xa) is encompassed by the present invention.

The FVIII mini-Ad of the present invention comprises the human FVIII cDNA as well as DNA elements (i.e., homologous recombination arms, AAV/ITR sequences, and a transcriptional control region) that support gene integration into a host cell genome and expression of human FVIII within a host cell. The viral proteins required for the DNA replication and encapsidation of the FVIII mini-Ad vector are provided in trans from a helper Ad (trans-complementation). To generate a relatively pure preparation of the FVIII mini-Ad vectors as viral particles, packaging of the helper Ad genome is attenuated by modification of its packaging signal. This allows for preferential packaging of the FVIII mini-Ad vector genome in the helper cell line.

In one embodiment, the FVIII mini-Ad comprises a site-specific integration mechanism. The mechanism may comprise a homologous recombination sequence or an AAV ITR targeted to a human integration sequence (AAVS1 site). In order to test the integration mechanism, the AAVS1 site must be transferred into the mouse genome. One method with this may be accomplished in through the use of transgenic technology such as embryonic stem cell transformation or by direct DNA injection of a transgene comprising the AAVS1 site into the male pronucleus of a mouse single-cell ova (57-60). A transgenic mouse developed by such methodology may be utilized to test the integration efficiency and specificity of the mini-Ad vector.

A further embodiment of the present invention addresses the potential anti-human FVIII immune response that may occur within a host animal. Such an immune response may interfere with utilization of the mini-Ad vector or analysis of the efficiency of the vector. Since the mini-Ad will deliver and drive expression of human FVIII within a mouse, an immune response of the treated mouse may complicate assessment of the duration and level of the FVIII expression. For these and other reasons, an FVIII-deficient transgenic mouse model is provided by the present invention. It is to be understood that the present invention comprises a non-human transgenic animal into which has been transferred a reporter or effector gene of a mini-Ad vector such that the animal is tolerized to the gene product of the reporter or effector gene.

Examples of each of the above-described transgenic mouse models are provided by the present invention and generated through microinjection of genetically-modified embryonic stem (ES) cells. One embodiment of the present invention provides a non-human transgenic animal incorporating a double stranded human FVIII DNA sequence operably linked to developmentally regulated cis-acting control elements to allow transient gene expression. The promoter may be derived from any developmentally regulated genetic unit capable of directing the expression of the human FVIII gene sequence transiently during development such that tolerization to the expressed exogenous protein occurs. Following tolerization and maturation of the animal, the FVIII transgene is no longer expressed. In the present invention, the α-fetoprotein promoter, was operably linked to the human FVIII cDNA and utilized to generate a transgenic mouse harboring the human FVIII cDNA within its genome. Such mice express hFVIII in a developmentally regulated manner, and as such, are tolerized to human FVIII. Another model comprises a transgenic animal containing the double stranded AAVS1 sequence which is the target for site specific integration of adeno-associated virus (AAV) gene therapy vectors. The human FVIII and AAVS1 transgenic animals can be bred to hemophiliac animals to create human factor FVIII tolerized hemophiliac animal models.

The following examples illustrate particular embodiments of the present invention and are not limiting of the specification and claims in any way.

### EXAMPLES

### Example 1

### Construction and Characterization of the Packaging-Signal Mutated Helper Ad and Mini-Ad Vectors That Carry Green Fluorescence Protein (GFP) Reporter Gene

1.1 Generation of the Packaging-Signal Mutated Helper Ad

Several packaging signal deletion-mutants of Ad5 have been described (90). Mutant dl10/28 (also described as dl309-194/243:274/358) contains a deletion between nt 194 to 243 and between 274 to 358 of Ad5. dl10/28 virus was generated by the method of Stow (89) by ligation of a plasmid containing the left end of Ad5 with this double mutation (pE1A-10/28) and the rest of Ad5 genome (90). dl10/28 showed a 143-fold decrease in virus yield in a single virus infection and, when co-infected with wild type virus, was not detected. We reasoned that with a helper virus containing the same mutation as dl10/28 we should be able to amplify the virus, although at low yields, and in the presence of mini-viral vector containing the wild type packaging signal the helper virus should remain unpacked.

The packaging signal was amplified by PCR from pE1A-10/28 using the following primers:

| | | |
|---|---|---|
| R7: | 5′-GGAACACATGTAAGCGACGG | (SEQ ID NO: 3) |

(nt 137 to 163 of Ad5 with AflIII site underlined)
and

| | | |
|---|---|---|
| R8: | 5′-CCATCGATAATAATAAAACGCCAACTTTG | (SEQ ID NO: 4) |
| | ACCCG | |

(nt 449 to 421 with Cla I site attached).

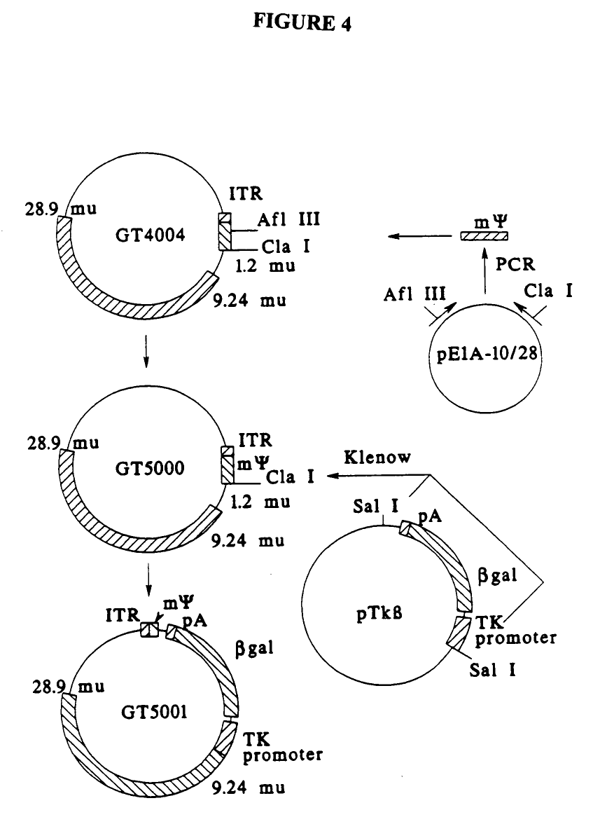

The amplified 133 bp fragment was cut with AflIII and ClaI and used to substitute the corresponding sequence of the shuttle vector GT4004 (see FIG. 4 for this construction scheme). GT4004 derives from pXCX2 (91) by extending the Ad5 left region from XhoI site (nt 5792, 16 mu) until SnaBI site (nt 10307, 28 mu), therefore GT4004 contains the left end of Ad5 from 0 mu to 1.2 mu with the Afl III site at 0.38 mu, an E1 deletion from 1.2 mu to 9.2 mu with Cla I site in this deletion point and the rest of the left arm of Ad5 until 28 mu. This extended left arm increases the frequency of homologous recombination used to generate recombinant virus. GT4004 with the wild type packaging signal substituted by the deleted one was named as GT5000. The β-gal expression cassette from pTkβ (Clontech, Ca.) was cut as a SalI fragment, blunted with Klenow enzyme and inserted into the blunted ClaI site of GT5000. The resulting plasmid, GT5001, contains therefore the double-deleted packaging signal and the E1 region of Ad5 replaced by the B-gal gene driven by the Tk promoter (FIG. 4). This construct allows for detection of helper virus by X-gal staining.

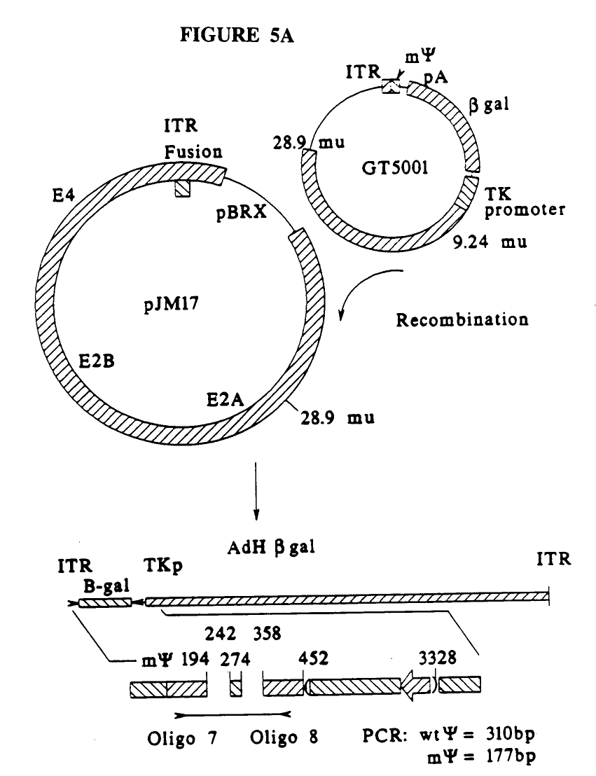

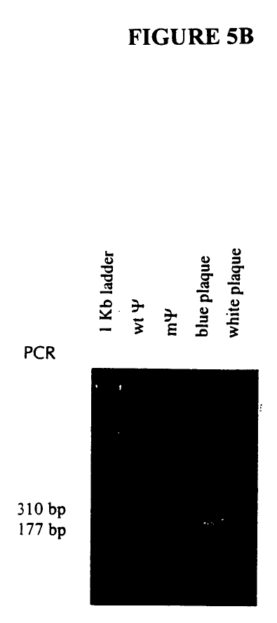

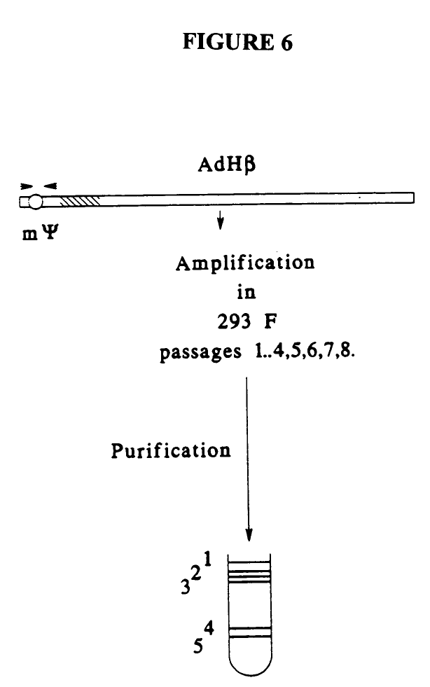

To generate the helper virus the method described by Graham and Prevec was used (91) (FIG. 5). An early passage of 293 cells obtained from ATCC, were grown in MEM-10% Horse Serum and seeded in 60 mm plates. At 30% confluence cells were cotransfected by CaPO₄ using 2 mg of GT5001 and 4 mg of pJM17 (91) per plate. Three days after cotransfection cells were overlaid with medium containing 0.5% agarose and thereafter the medium above the overlay was changed every-other day. When plaques became visible, X-gal (40 mg/ml in DMSO) was directly added to the medium to 100 mg/ml an let incubating overnight. Plaques producing the desired helper virus were identified by the blue color (FIG. 6). Blue plaques were fished and the agarose plugs were resuspended in 1 ml MEM-10% FBS. 370 ml of this medium were processed for PCR amplification of the packaging signal as follows: 40 ml of 10× DNase I buffer (400 mM Tris-HCl pH 7.5, 60 mM Cl₂Mg, 20 mM Cl₂Ca) (as a control of the treatment a tube with 0.5 mg of the shuttle vector was used) and 1 ml of DNase I (Boehringer M, 10 u/ml) were added and incubated for 1 h at 37° C. DNase I was inactivated and viral capsids were opened by adding: 32 ml EDTA (0.25 M), EGTA (0.25 M), 10 ml SDS (20%), 5 ml Proteinase K (16 mg/ml) and incubating at 56° C. for 2 h. After one phenol:chlorophorm:isoamyl alcohol (1:1:1/24) extraction, 1 ml of yeast tRNA (10 mg/ml) was added to help precipitation of viral DNA which was collected by centrifugation at 12000 rpm in a microcentrifuge and resuspended in 20 ml of H₂O. 5 ml were used for a PCR reaction with primers R7 and R8.

One blue plaque with the desired deleted packaging signal was further amplified in 293 cells grown in DMEM-10% FBS. This helper Ad is hereafter termed “AdHelper-βgal” or “AdHβ”. The virus was extracted at 48 h post-infection by centrifugation of the collected cells at 800 g for 5 min and three cycles of quick freeze and thaw of the cell pellet. This crude extract from X cells was used to infect 3× cells (amplification scale was 1 to 3 in contrast to 1 to 20 for a virus with wild type packaging signal) and plaques identified by staining with X-gal. At every passage the deleted size of the packaging signal was verified by PCR of the supernatant. This deletion and the β-gal expression were stable in all the passages analyzed. At passage 9 AdHβwas purified by CsCl. Purification was done by three cycles of freeze-thawing, layering the lysate onto a step gradient of 0.5 ml CsCl 1.5 mg/ml+2.5 ml CsCl 1.35 mg/ml+2.5 ml CsCl 1.25 mg/ml, and centrifuging in a SW41 Beckman rotor at 10° C., 35000 rpm, 1 h. The collected virus band was mixed with CsCl 1.35 mg/ml and centrifuged for 18 h as before. The virus band was dialyzed twice against PBS and once against PBS-10% glycerol, and stored at −80° C. Five different bands were seen after the second gradient and every one was purified separately. Viral DNA was extracted from purified virus by EDTA/SDS/Proteinase K treatment, phenol/chloroform extraction, and ethanol precipitation (same conditions as described above for PCR). In ethidium bromide gels, no DNA was detected in the three upper bands and were considered to be mostly empty capsids. The two lower bands contained viral DNA and therefore were full capsids that, by restriction map analysis, were shown to correspond with AdHβ. By PCR with R7+R8 oligos, the deleted packaging signal was amplified from all bands, indicating that virus contained the desired attenuation. With this purified helper virus we test the packaging of different mini-viral vectors. To determine the titer, which is expressed as a number of plaque forming units (PFU) per milliliter of virus in solution, the virus-containing solution was serially diluted in D-MEM 10% FBS (1:10 dilution until 10⁻¹²) and used to infect 293 cells at 90% confluence (0.5 ml/well in 6 well-plates). After 1 h infection at 37° C., the viral suspension was replaced by fresh medium. The next day, cells were overlaid with medium containing 0.5% agarose, 0.025% yeast extract and 5 mM Hepes pH 7.4. Plaques were counted after 6 to 10 days. The titer obtained after amplification and purification of AdHβ was about 10⁹ PFU/ml (virus purified from 20 plates of 150 mm² and resuspended in a final volume of 1 ml). This titer is about 100× lower than that obtained with a similar viral vector containing the wt packaging signal.

1.2 Construction of the Plasmids for the Mini-Viral Vectors.

It has been shown that the linear adenovirus DNA, when covalently circularized head-to tail by its terminal ITRs can be grown as a plasmid in bacteria but it will replicate and produce virus when transferred into permissive human cells (92). Functional junctions have been naturally selected by transforming bacteria with circular DNA extracted from infected cells. Small deletions in the joints were observed which presumably conferred stability to the plasmids by destroying the perfect palindrome that would result from the head-to-tail fusion of the ITRs of adenovirus DNA. The basic minivirus structure is therefore a plasmid that contains the left end of Ad5 (including the 103 nt-ITR and the packaging signal until nt 358) fused to the right end of Ad5 (at least including the 103 nt-ITR). The initial approach used to test the mini-viral vector system included the generation of progressive deletions in plasmid pJM17 that contains a functional ITR fusion. pJM17 is a plasmid that contains the entire genome of Ad5 as a DNA molecule circularized at the ITR sequences and a pBR322 derivative, pBRX, inserted in E1A (providing the bacterial replication origin and ampicilin and tetracycline resistant genes) (93). When transfected in 293 cells, which complement the E1A defect, pJM17 replicates but is not packaged because is too large (40.3 kb) to be packaged into the adenovirus capsid (maximum is 38 kb).

Examples of various mini-viral vectors demonstrated in the current literature as well as that of the present invention are illustrated in FIG. 1. pJM17 was cut with AscI and religated obtaining pBRX-AscI. This removed from mu 43.5 to 70.2 of Ad5 which completely deletes E2A (DNA binding protein) and L3 (hexon, hexon-associated proteins and 23K protease), and partially deletes L2 (penton base and core proteins) and L4 (hexon-associated protein, hexon-trimer scaffold protein, and 33K protein). This deletion abrogates replication and capsid formation from the circular viral DNA, rendering it completely dependent on a helper virus that provides in trans a sufficient quantity of the required replication proteins. pBRX-AscI contains a unique Spe I site at 75.2 mu (L4) into which a 2.7 kb DNA fragment comprising a green fluorescence-protein (GFP) expression cassette was inserted to give M32 (Minivirus of 32 kB). This GFP-cassette is composed of a CMV enhancer/β-actin promoter (CA promoter), the Aequorea victoria GFP cDNA, and a SV40 polyA signal. The use of GFP in the mini-viral vector constructs was utilized in order to determine the presence of the vector in cells using the fluorescence microscopy. Flourescent microscopy represents one of several methods including but not limited to flow cytometry that may be utilized to detect cells expressing GFP. The presence of AdHβ can be detected by the blue color of X-gal staining. To generate M31, M32 was cut with MluI and religated, this removes from 31.4 to 34.5 mu which partially deletes L1 (52K, 55K and penton-associated proteins). To generate M28, M32 was cut with MluI and AscI and religated, this removes from 31.4 to 43.5 mu which completely deletes L1 and the L2 portion that still remained in M32. To generate M26, M28 was cut with Rsr II and Spe I and religated, this removes from 30.9 to 75.2 mu extending the L1 and L4 deletions. To generate M23, M32 was digested with Nsi I and religated. The Nsi I fragment from 32.2 mu to the CA promoter (with a NsiI site next to the fusion with 75.2 mu), containing the GFP cassette, was religated so the Nsi I site of the CA promoter ligated to 5.5 mu and the Nsi I site at 32.2 ligated at 75.3 mu. This abrogates expression of all proteins between 5.5 to 75.3 mu including E2b (terminal protein, DNA polymerase) and IVa2 proteins. To generate M20, M23 was cut with Mlu I and Asc I, which removes the region from 34.5 to 43.5 mu of the Nsi I fragment of M23, and religated.

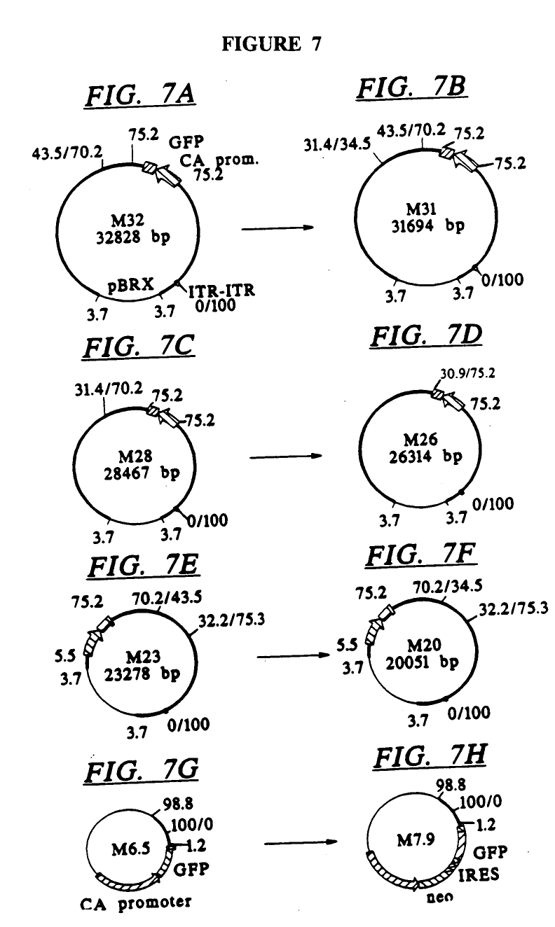

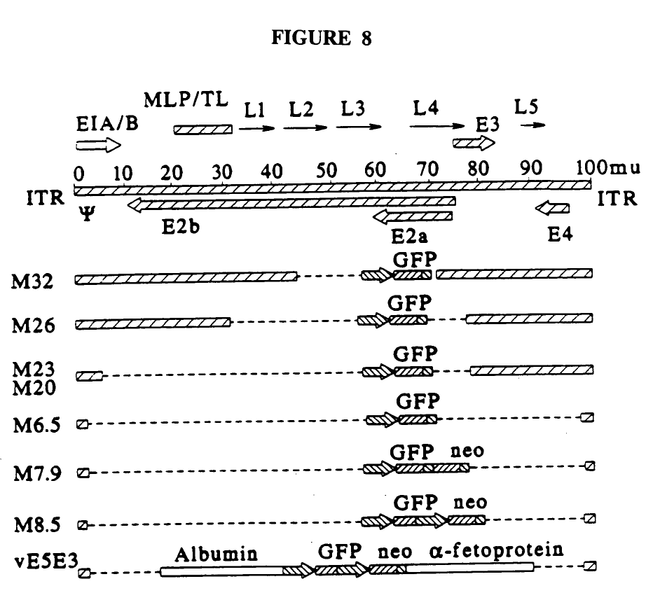

Instead of trimming down a circularized full-length viral genome such as that in pJM17, other mini-viral vectors were constructed by subcloning the minimal cis elements necessary for replication and packaging, including the ITR sequences and the packaging signal, into a small plasmid such as pBluescript (Stratagene) and progressively adding the transgene cassettes and other elements that could improve the therapeutic potential of the viral vector such as elements for episomal maintenance or chromosomal integration (FIGS. 3B and 7, bottom). The head-to-tail fused ITRs and the packaging signal next to the left ITR (ITR ITR+pac) were cut from pBRX-AscI with Eco47III (98.7 mu) and PvuII (1.26 mu) blunted and subcloned into SmaI-EcoR V of pBluescript, respectively. The resulting plasmid, pBS/MiniITR or GT4007, is a 3.8 minivirus plasmid with no expression cassette and several unique restriction sites flanking the ITR/ITR+pac. Using Xho I and Kpn I, the GFP-expression cassette described above was subcloned into pBS/MiniITR to generate M6.5. An internal ribosome entry site (IRES) and a neomycin (neo) cDNA were then subcloned between the CA promoter and the GFP gene to produce M7.9. A similar minivirus was generated comprising neo and GFP in two separate cassettes, M8.5: the Nru I-BstE II fragment from pREP9 (Invitrogen) containing the Tk promoter, neo cDNA and Tk pA, was blunted and subcloned into Stu I-EcoR I of M6.5. M8.5 was used to construct a larger miniAd plasmid in order to test the packaging of miniAd vector with a complete substitution of the adenoviral genome by exogenous DNA. As inserts we used genomic fragments corresponding to the 3′ half of the albumin gene and the 5′ half of the alpha-fetoprotein gene. These fragments were chosen as potential arms with the prospect of studying homologous recombination in this site. We inserted these fragments upstream and downstream of the double GFP/neo expression cassette of M8.5. Therefore, in the resulting construct, pGnE5E3 (23.8 Kb), the GFP and neo transgenes substitute the corresponding 10 Kb albumin-fetoprotein intergenic region present in the human genome. FIG. 8 shows the structure of the minivirus to be obtained with the plasmids described above.

1.3 Generation and Amplification of the Mini-AdGFP Vectors.

AdHβ was utilized to support the replication and packaging of the various mini-Ad plasmids. It was important to determine whether the minivirus could be packaged. It was also important to determine whether the size of the minivirus affected the packaging efficiency.

In adenovirus, 100% of the wild type length of DNA is most efficiently packaged, and as the genomic size increases to a maximum of 105% or decreases below 100%, packaging becomes less efficient. A lower limit of 69% (25 kb) has been suggested (94) when wild type adenovirus was used to complement the defective minivirus, but the use of an attenuated helper virus allowed the amplification of a shorter minivirus.

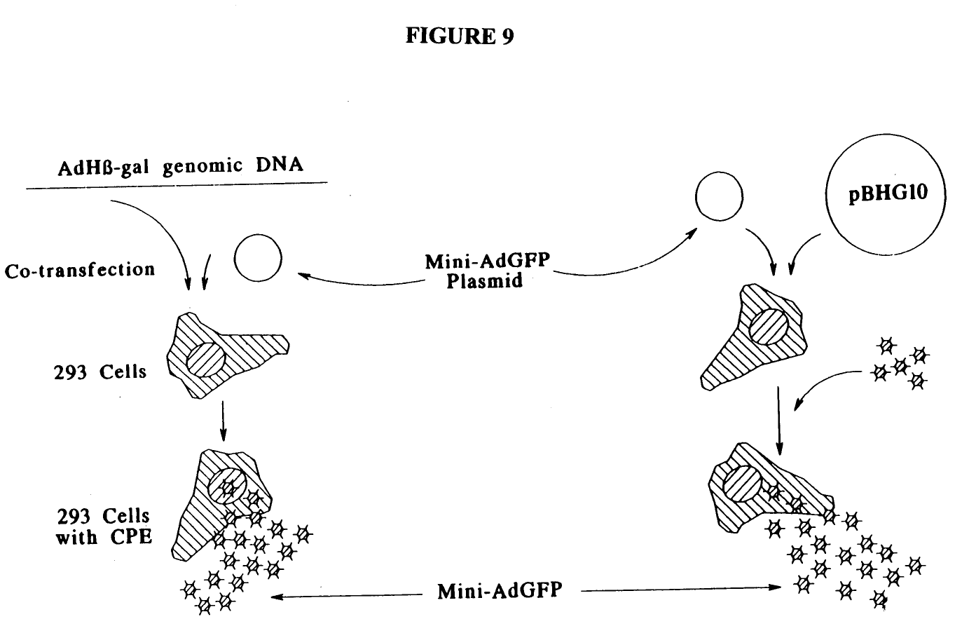

To complement the mini-viruses of the present invention, two methods were utilized that each function with a similar efficacy FIG. 9. In the first method, a CsCl-purified minivirus plasmid was cotransfected with the linear viral DNA extracted from purified AdHβ. Note that the method utilized to purify the viral DNA is subjected to SDS and Proteinase K which destroys the terminal protein responsible for priming replication. This method was utilized to avoid giving the helper virus a replicative advantage over the minivirus plasmid which also lacks the terminal protein. Accordingly, complementation by direct infection with AdHβ did not rescue minivirus. Cotransfection was accomplished using Ca₂PO₄ and 2 mg of mini-viral plasmid and 1 mg of viral DNA per well in a 6 well-plate with 293 cells at 50% confluence. After an overnight incubation in the transfection mixture., the medium was changed and the efficiency of transfection was assessed by examination of cells using fluorescence microscopy. With CsCl-purified plasmids this efficiency reached 100% irrespective of the size of the plasmids. Six days post-cotransfection, CPE was observed and virus was harvested from the cells by three cycles of freeze and thaw. In the second method of complementation the minivirus plasmid was cotransfected with pBHG10, a circularized adenovirus plasmid similar to pJM17 incapable of being packaged due to a complete deletion of the packaging signal (95). This plasmid produces all the early proteins necessary for replication as well as the late proteins that form the capsid. When the minivirus is present in the same cell as pBHG10, it will also replicate and, as the minivirus contains the wild-type packaging signal, the miniviral vector will be the major nucleic acid encapsidated. However, when the minivirus is released to the neighbor cells it will not be amplified because is defective. Therefore, to amplify the minivirus, three days after the cotransfection, the cell monolayer was infected with AdHβ at a multiplicity of infection (moi) of 10 plaque forming units (pfu)/cell. Three days after co-transfection, CPE was observed and virus was harvested by three cycles of freeze and thaw.

Regardless of the method of complementation, the lysate (passage 0 of the produced minivirus) was used to infect a fresh monolayer of 90% confluent 293 cells (using 1 to 3 amplification scale). The day after infection, the presence of minivirus was observed by fluorescence and the presence of helper confirmed by X-gal staining. If any helper virus was present in the lysate, further incubation of the cells would lead to the amplification of the mini-virus+helper mixture with the appearance of CPE (the new lysate of this monolayer will be considered as passage 1 of the minivirus). If no helper was present in the lysate, the minivirus alone would not be packaged and only by the addition of new helper would the CPE appear. Therefore the presence of the helper was assessed by X-gal staining and, with much higher sensitivity, by the appearance of CPE.

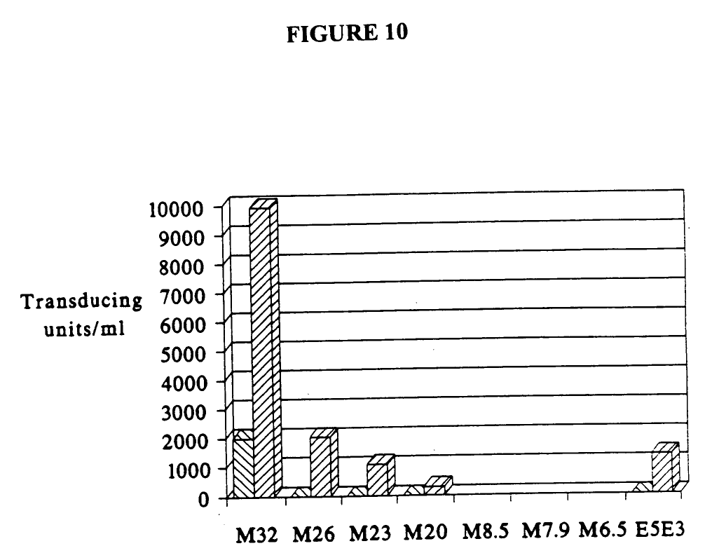

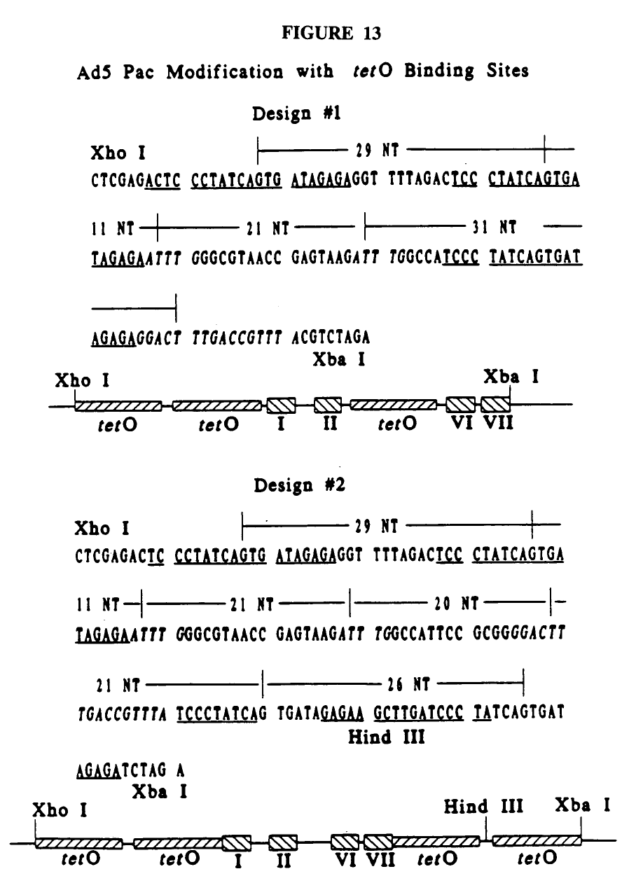

Following separate transfections with each of the minivirus constructs (M32, M31, M28, M26, M23, and M20), the appearance of plaques with GFP was observed by fluorescence microscopy. This indicated that the complementation was possible for each of the plasmids tested. Following infection of fresh 293 cells monolayers with the crude extracts of each virus, CPE was observed after 2 days indicating the presence of helper virus. The results of further passage of the minivirus demonstrated that in every passage a 5-fold amplification was produced, and that the packaging efficiency was proportional to the minivirus size (FIG. 10). A drop in efficiency of 2 fold per every 3 kb decreased vector size was observed. For example, the efficiency of packaging of M20 would be 2.48% of the wild type (being (36−x kb)/3=n the efficiency is 0.5ⁿ). However, with M6.5, M7.9 and M8.5 no fluorescent plaques were found, indicating very inefficient or absent packaging (FIG. 13). This could reflect a packaging lower limit somewhere between 8.5 Kb and 20 Kb. However, it seems more probable that packaging still might take place between these limits but, according to the linear decrease observed, the 11.5 Kb size difference would result in a 7.6 fold less packaging efficiency and amplification may not then be possible.

Complete substitution of the viral genome by exogenous DNA was possible and whether this would affect the packaging efficiency was tested. A completely substituted miniAd of 23.8 Kb containing only the ITRs and the packaging signal of Ad5 was constructed. As exogenous DNA two expression cassettes in tandem, one for GFP and another one for neomycin, were flanqued by two long arms of albumin and an α-fetoprotein genomic DNA (FIG. 9). Packaging was demonstrated by the increasing number of GFP-positive 293 cells when the virus obtained after an initial complementation with AdHb v DNA was passed. This transducing units titer was similar to that obtained with M23 (FIG. 10), indicating that exogenous DNA did not have a detrimental effect in the packaging efficiency when compared to Ad5 DNA.

1.4 Purification of the Mini-AdGFP Virus (M32)

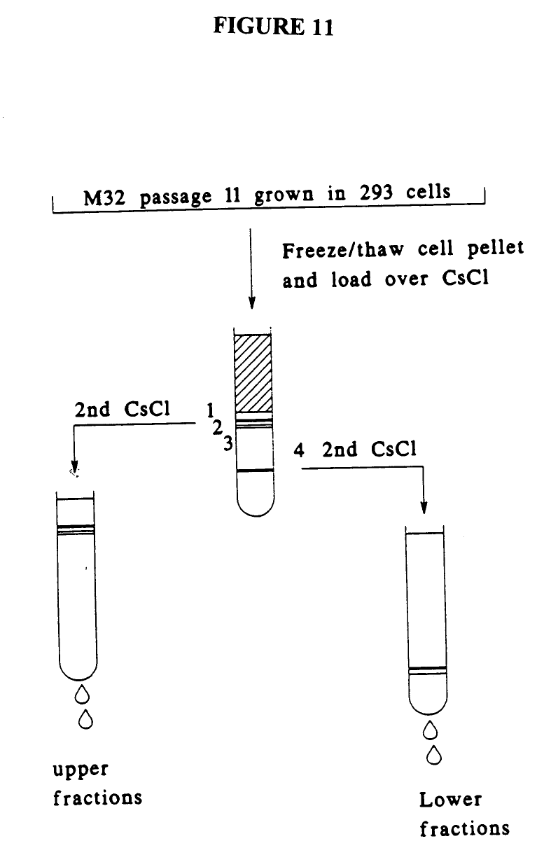

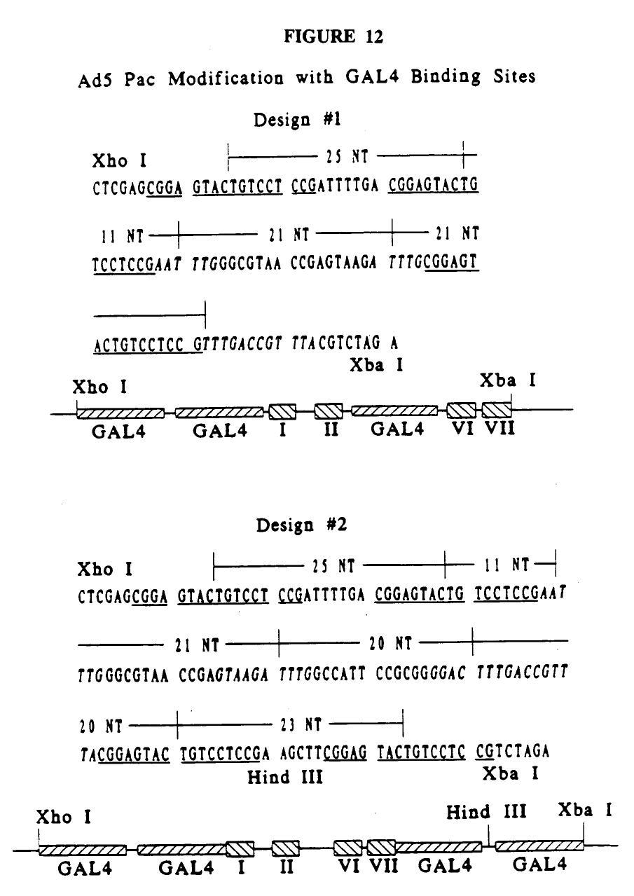

Following passage of the minivirus, the titer increased until all cells became fluorescent following infection. This occurred, for example, at passage 4 of M32. When passage 8 was reached by continuously passing M32 at 1 to 3 amplification scale, enough virus was obtained to infect 75 plates of 150 mm². When CPE was apparent, the virus was extracted by three freeze/thaw cycles and purified by a CsCl gradient as described above. In the gradient four bands were observed, three upper (and therefore lighter) bands and one thicker band in the middle of the centrifuge tube (see scheme in FIG. 11). Every band was collected separately by aspiration from the top of the tube, and dialyzed. Infection of 293 cells with every band and fluorescence observation or X-gal staining demonstrated that the mini-virus and helper virus were both present on the higher density band. Based on the number of green and blue cells, the amount of minivirus and helper was determined to be within the same range. The different size of the viral DNA present in M32 (32 Kb) and in AdHβ (37.1 Kb) should make M32 slightly less dense than AdHβ. To increase the mini-Ad vector to helper ratio the higher density band was separated in a 1.35 g/ml continuous CsCl gradient and fractions were collected from the bottom of the tube. 0.5 ml of every fraction was used directly to infect 293 cells at sub-confluency to check for fluorescent and blue cells after 24 h.

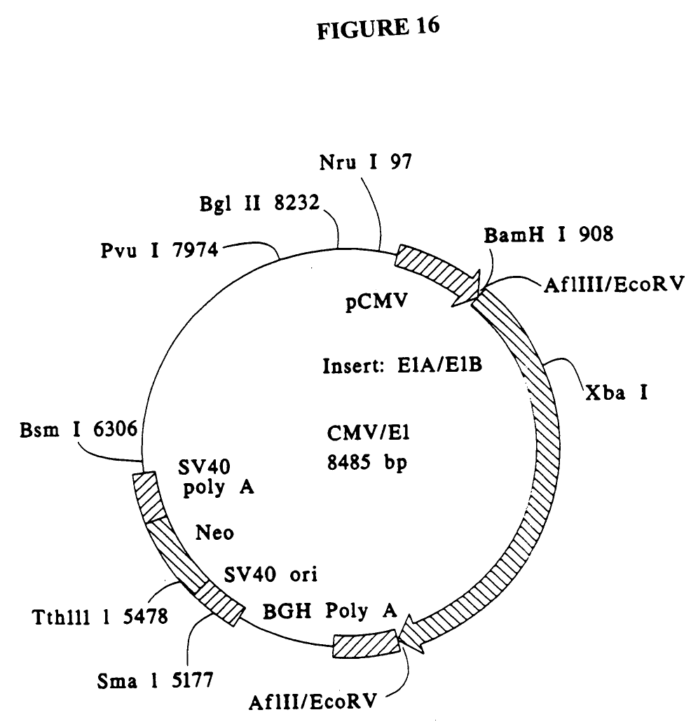

An aliquot of 0.5 μl of every fraction was used to infect one well of a 96 well/plate with 293 cells at 60% confluency. Initial fractions (1 to 6) did not contain M32 or AdHβ (these fractions represent up to 3 ml of the gradient). 100 μl samples of fractions 7 to 16 reveal a large amount of M32 and AdHβ (see panel B for β-gal staining of the same fractions shown under fluorescence in panel A). Subsequent fractions (17 to 29) show a level of M32 similar to the previous fractions but the level of AdHβ is approximately 10 times lower. Therefore, fractions 17-29 represent a 10-fold enrichment of M32 with respect to AdHβ. Following a peak containing large amounts of AdHβ (FIG. 16, fractions 7 to 16), a lighter fraction followed that revealed a 10-fold enrichment for M32 with respect to AdHβ (fractions 17 to 29). Fractionation through CsCl may therefore be utilized to decrease the amount of helper virus present in the mini-Ad preparations.

In summary, the results indicate that the helper used with the partial deletion in the packaging signal taken from the dl18/28 virus is able to complement the large deletions in the mini-viral vector system but it is still packaged in the presence of minivirus. This helper can be used when a pure population of minivirus is not critical, for example in an antitumoral vector system where a minivirus containing several therapeutic genes (for example, interleukins and tumor-suppresser genes) can be combined with this helper containing another therapeutic gene. When higher mimi-Ad to helper ratio is required, this helper needs to be further attenuated in its packaging.

### Example 2

### Design of Packaging-Signal Interfered Helper Ad

Since the packaging of adenovirus requires packaging proteins to bind the packaging elements (A repeats) (90, 96), and this invention introduces several specific DNA binding sequences adjacent to Ad5 packaging signals (A repeats) to further physically interfere helper virus packaging function. Two DNA binding sequences have been chosen: A. GAL 4 binding sequence (97); B. tetracycline operator sequence (tetO) (98, 99). GAL4 is a sequence-specific DNA-binding protein that activates transcription in the yeast Saccharomyces cerevisiae. The first 147 amino acids of GAL4 binds to four sites in the galactose upstream activating region UAS_{G} or a near consensus of the naturally occurring sites, the “17-mer” 5′-CGGAGTACTGTCCTCCG-3′ or 5′-CGGAGGACTGTCCTCCG-3′ (97). tetO comes from the Tn10-specified tetracycline-resistance operon of E. coli, in which transcription of resistance-mediating genes is negatively regulated by the tetracycline repressor (tet R) which binds a 19-bp inverted repeat sequence 5′-TCCCTATCAGTGATAGAGA-3′ in tet O (98, 99).

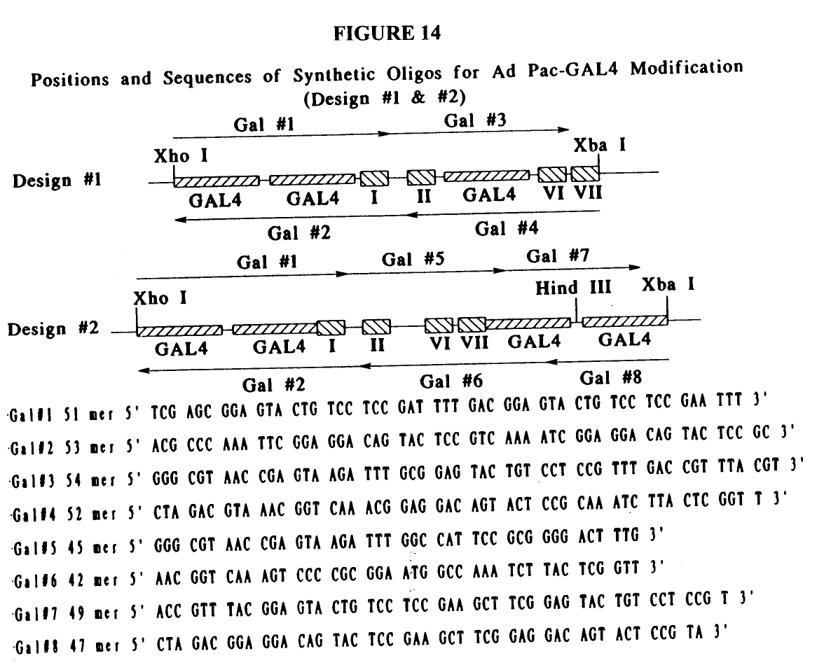

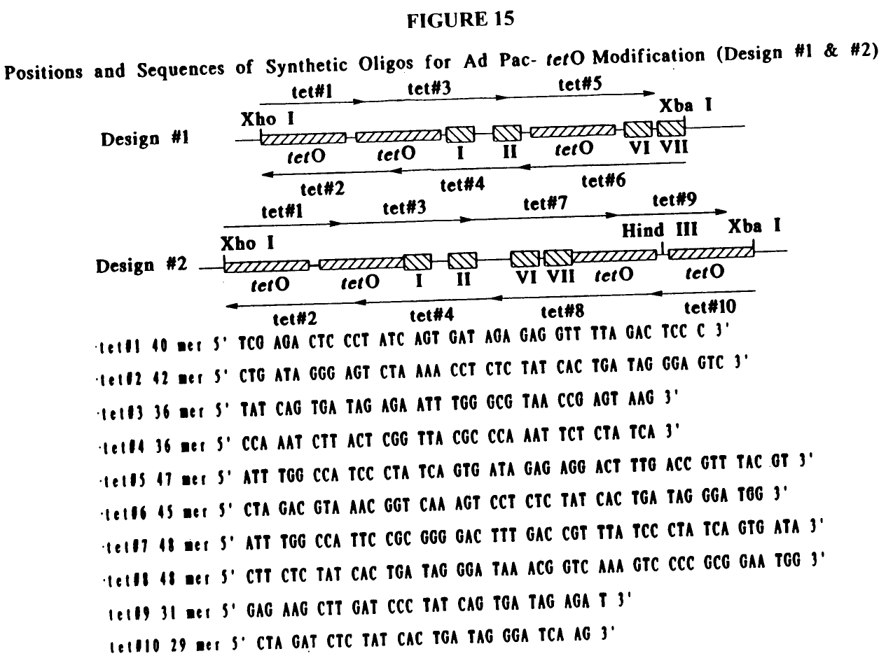

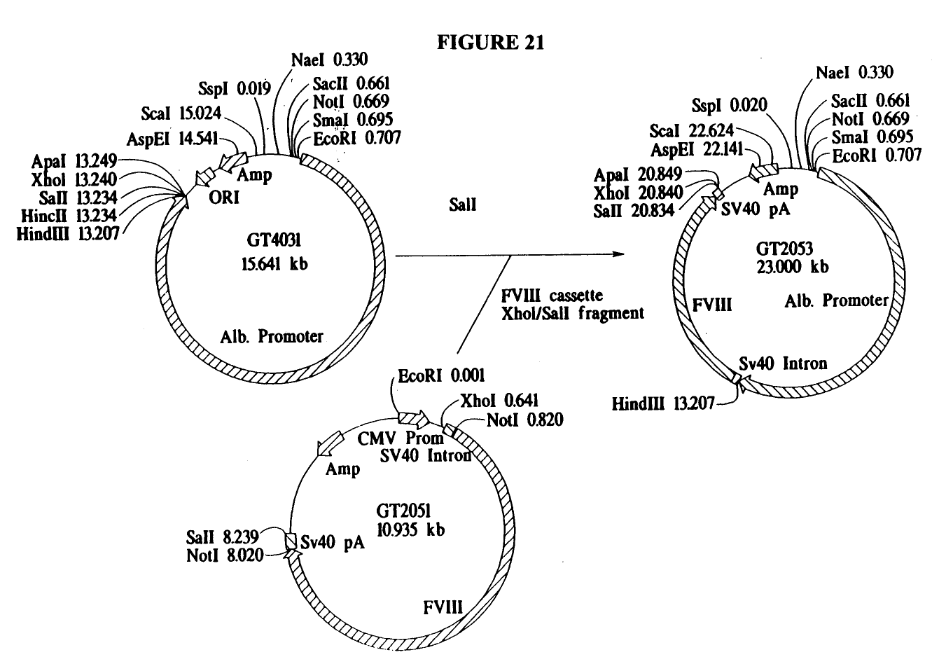

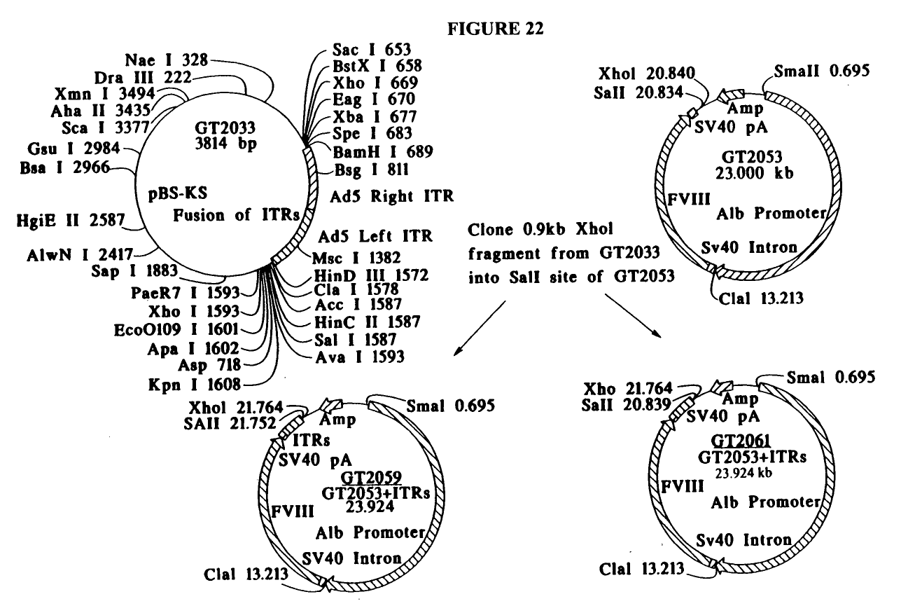

Based on the packaging signal mutation construct GT5000 (Example 1, section 1), a synthetic sequence has been utilized to replace the sequence between Xho I and Xba I (nt 194, 0.5 mu to nt 452, 1.25 mu) of GT5000. Four synthetic sequences (FIGS. 21 and 22) have been designed. All four synthetic sequence contain the Ad5 packaging element (A repeats) I, II, VI and VII. Three or four repeats of 17-mer GAL4 binding sequences (5′-CGGAGTACTGTCCTCCG-3′) (97) or 19-mer tet sequences (5′-TCCCTATCAGTGATAGAGA-3′) (100, 102) were introduced around or between these A repeats (FIGS. 13 and 14). Since the region between A repeats can affect packaging efficiency (90, 96), the distance between each A repeat is maintained as nearly integral turns of the helix, i.e. 10, 21 or 31 bp. FIGS. 14 and 15 show the synthetic oligo sequences and positions.

### Example 3

### Construction and Characterization of Ad-E1 Helper Cell Lines

The majority of adenoviral vectors used in gene therapy applications were designed to have deletions in the E1 region of the adenovirus 5 (Ad5) genome. The E1 region, not including region IX, consists of 9% of the left end of Ad5 (1.2-9.8 map units), and encodes two early region proteins, E1A and E1B. Expression of E1A/E1B is required for virus replication and for expression of all other Ad5 proteins such as E2-E4 and late proteins (100). Deletion of E1 creates a replication-incompetent virus that, in theory, is silent for expression of all Ad5 proteins and expresses only the transgene of interest. Deletion of E1A and E1B is also of interest for safety reasons, since these two proteins, in combination, have been implicated in oncogenic transformation of mammalian cells (101-103). All of the Class I adenovirus vectors used to date in human clinical trials, as well as, the novel packaging-deficient helper virus described in Example 1 are deleted for E1.

E1-deficient adenoviral vectors are propagated in an Ad5 helper cell line called 293 (104). 293 cells were derived by transforming human embryonic kidney cells with sheared fragments of Ad5 DNA. Genomic analysis revealed that 293 cells contain four to five copies per cell of the left 12% of the viral genome (including the entire E1 region) and approximately one copy per cell of 9% of the right end, the E4 region (105). While 293 cells are very efficient at producing high titers of E1-deficient adenovirus, they have the disadvantage that, due to the presence of extraneous Ad5 sequences integrated into the 293 genome (other than the E1 region), recombination can occur with sequences in the E1-deficient adenovirus vector causing the production of E1-containing, replication-competent adenovirus (RCA). Depending on how early a passage the aberrant recombination event occurs during the amplification and propagation of the E1-deficient adenovirus, and which passage is used for large-scale production of the adenovirus stock, production of RCA in 293 cells can present severe ramifications for the safety of human gene therapy trials (106). In addition to production of RCA, recombination in 293 cells can also cause deletions and rearrangements that effect transgene expression, thereby decreasing the titer of functional adenovirus particles. Recently, cell lines have been developed using defined Ad5 DNA fragments, including the E1 region, however these cell lines retain significant sequence overlap with homologous sequences in the E1-deleted adenovirus vectors, which allows for undesirable homologous recombination events and the possibility for generation of RCA (107, 108).

3.1 Construction of Plasmids for Cell Line Generation

To eliminate the possibility of recombination with the adenoviral vector, a novel Ad5 helper cell line has been developed which harbors only the E1A/E1B sequences required for complementation, and does not contain any homologous sequences that overlap with regions in the E1-deficient adenovirus. A 3.1 kb DNA fragment between AfI III (462 bp) and AfI II (3537 bp) sites, which contains sequences encoding for Ad5 E1A and E1B, was cloned in two pieces, sequentially, into the superlinker vector, pSL301 (Invitrogen), as follows: First, an 881 bp Afl III to XbaI fragment (Ad5 bp 462-1343) was cloned from pBRXad5 KpnIC1 (a subclone of pJM17) into pSL301 (Afl III/XbaI). Second, a contiguous 2194 bp XbaI to Afl II (Ad5 bp 1343-3537) was cloned from pBRXad5XhoIC1 into the same vector. The resultant 3075 bp E1 fragment (in pSL301) contains the TATA box and RNA cap site for E1A, E1A coding sequence, complete E1B promoter, and E1B coding sequence, including the stop codon for E1B p55 protein, but not including region IX. The 3075 bp Afl III-Afl II E1A/E1B fragment (Ad5 bp 462-3537) was isolated, blunted with Klenow enzyme, and blunt-end ligated into the EcoRV site of the mammalian expression vector, pcDNA3 (Invitrogen), under control of the CMV promoter/enhancer. This process generated an Ad5E1 expression vector, CMV-E1 (FIG. 16).

3.2 Generation and Characterization of the New Cell Lines

The CMV-E1 expression vector (including the G418 resistance gene, neo) was transfected using Lipofectamine (Gibco/BRL) into A549 human lung carcinoma cells and G418^{R} colonies were isolated. Single-cell clones were screened for functional E1A/E1B expression; An E1-deleted adenovirus containing a green florescence protein (GFP) expression cassette under CMV/β-actin (CA) promoter, Ad5CA-GFP, was used to infect the A549-E1 clones. Three days post-infection, clones were screened for production of E1-complemented Ad5CA-GFP adenovirus by visual examination for cytopathic effect (CPE). One clone, A549E1-68, displayed 100% CPE in 3 days (similar to that observed for 293 cells). This clone also showed high infectivity, in that virtually 100% of the cells fluoresced green, as determined microscopically, 24 hrs. post-infection (FIG. 22). Infection with the E1-deleted adenovirus, Ad5CA-GFP generated a clear area in the center of its plaque, which is evidence of the CPE caused by E1-complemented virus amplification.

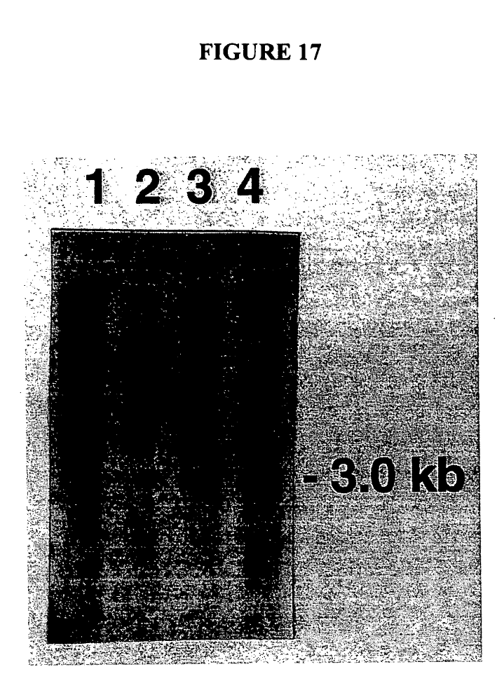

The high infection rate as well as rapid generation of CPE induced in this cell line is strong evidence that functional E1A/E1B proteins are being produced which are capable of promoting the replication and amplification of the E1-deleted Ad5CA-GFP virus. Southern Blot analysis using an E1 sequence-specific probe demonstrated the presence of the CMV-E1 transgene in A549E1-68, a subclone of A549E1-68 (E1-68.3), and 293 cells, but not in the parental A549 cell line (FIG. 17). The morphology of the E1-transfected cells was significantly different from the parental A549 cell line. A549 cells, at sub-confluent density, grow as distinct single cells with an elongated, fibroblast-like morphology, whereas, the E1 cell line, A549E1-68, grows as colonies of cells with a more cuboidal morphology.

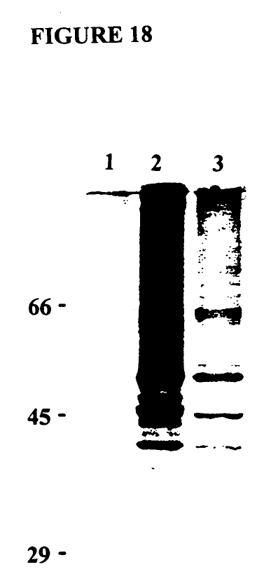

A549E1-68 was compared with 293 cells for production of E1-deleted adenovirus (Ad5CA-GFP) by plaque assay and found to produce an equivalent titer of complemented virus (7×10⁹ PFU for A549E1-68 vs. 9×10⁹ PFU for 293). Immunoprecipitation and Western blot analysis using an E1A specific antibody (M73, Oncogene Science), revealed two E1A-specific bands with apparent molecular weights of 46 kd and 42 kd, corresponding to products expected from E1A 13S and 12S mRNAs (6), and identical in size to those observed in 293 cells (FIG. 18). A549E1-68 produced a band of approximately 55 kd using a monoclonal Ab specific for E1B p55. This 55 kd, E1B-specific band, as well as secondary background bands, were observed in 293 cells also (FIG. 18). Extra “background” bands found in both experimental and control lanes have been observed by other authors and have been attributed to co-immunoprecipitation of a variety of proteins including, cyclins, p53, and Rb. Unlike A549E1-68 and 293 cells, the parental A549 cell line showed no expression of 46 kd, 42 kd, or 55 kd E1A/E1B proteins. It is clear that A549E1-68 not only expresses E1A and E1B, but that they are functional, since this cell line can complement for production of high titer, E1-deleted, recombinant adenovirus. To prove that this new Ad5 helper cell line can complement without production of RCA, we are serially passaging E1-deleted adenovirus on A549E1-68 cells and testing the virus amplified during passaging, on parental A549 cells for production of E1-containing, replication-competent adenovirus (RCA) by CPE, as well as by using PCR primers specific for E1A/E1B sequences. This cell line will be used during propagation and scale-up of all E1-deleted adenovirus vectors, to ensure that production lots are free of RCA.

The cells were further optimized and developed by a process of subcloning. Two subclones, A549E1-8 and A549E1-14, were selected. The doubling time of the subclones in serum-free culture is about 48 hr. The clone A549E1-8 was adapted to serum-free culture. Burst sizes of the A549E1-8 cells (in terms of vp/cell) in the culture conditions with or without serum were found to be comparable to that of 293 cells in conventional culture condition. The A549E1-8 cells were also successfully adapted into anchorage-independent culture for a production process using a suspension stirred-tank bioreactor. Consecutive and scale-up propagation of Ad5GFP in A549E1-8 cells was carried out to determine the frequency of RCA generation. Through monitoring numerous manual scale-up productions and more than three runs using the CellCube system for production, no RCA incidence has been found by a bioassay with the sensitivity of 1 RCA per 10⁹ pfu for the Ad5GFP vector.

### Example 4

### Expression Cassette Comprising the FVIII cDNA

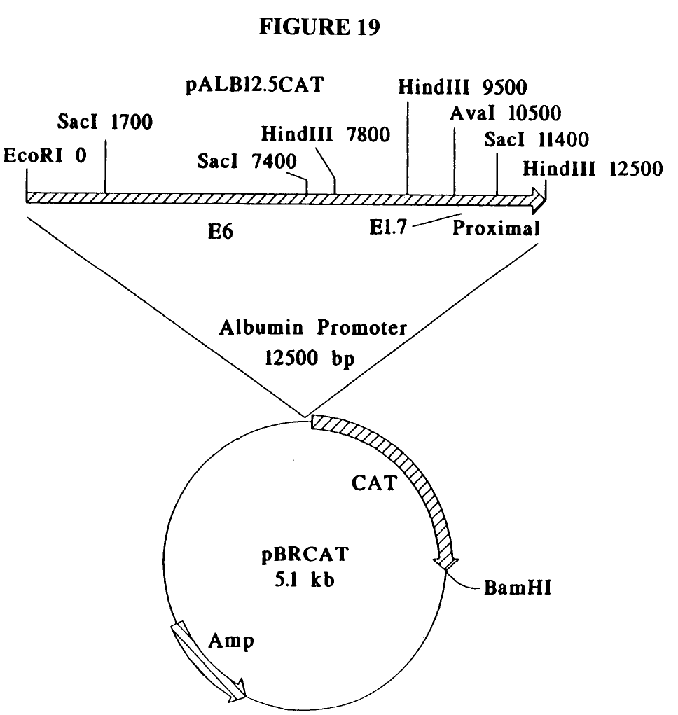

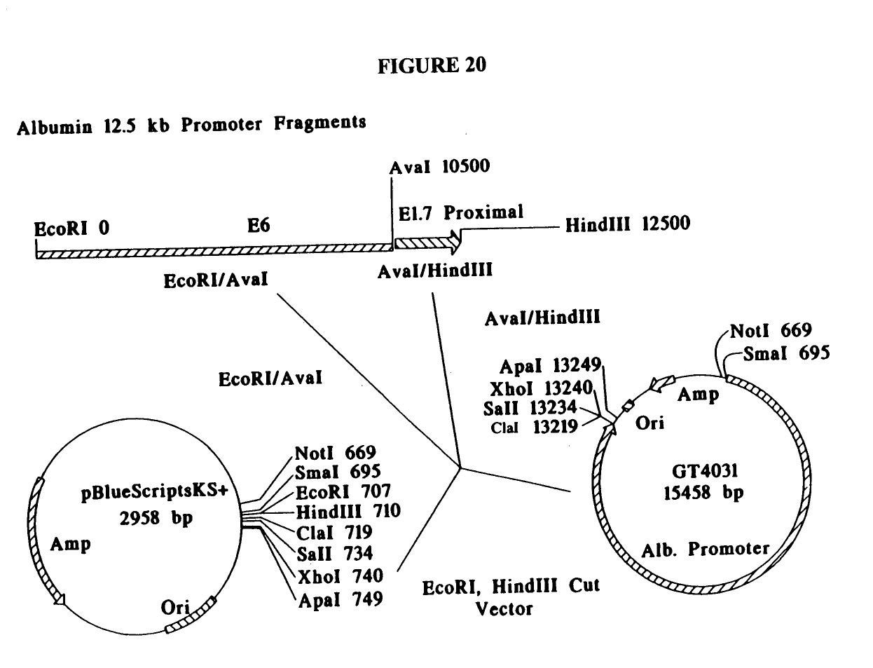

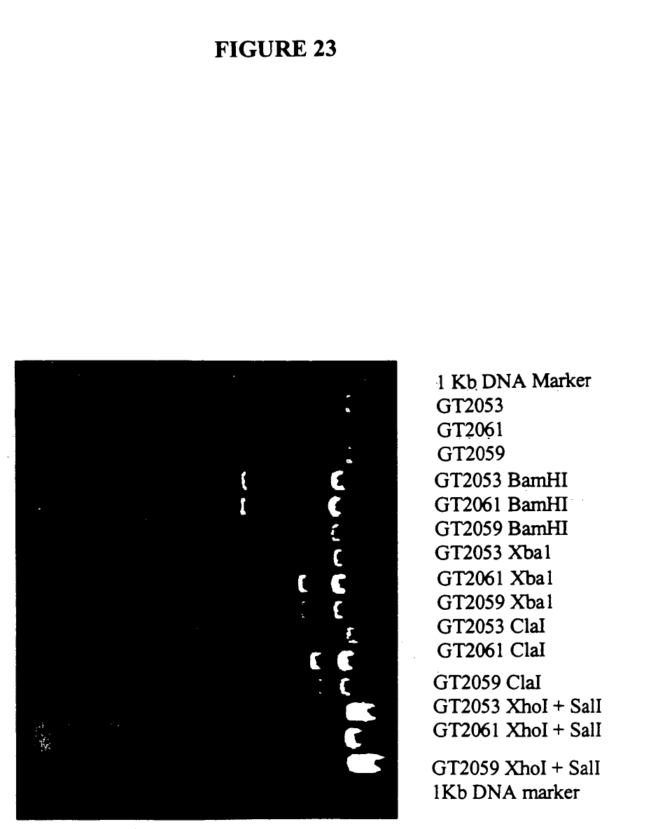

The large capacity of the Ad-mini vector of the present invention for the gene of interest allows for insertion of large promoter and protein coding regions that far exceed the size capacity of the conventional Ad vector. It is preferred, for the purposes of the present invention, that the FVIII mini-Ad vector delivers the FVIII gene to the liver. It is, therefore, preferred to utilize a highly active promoter that functions in the liver. One such promoter is the human albumin gene promoter (32). A 12.5 kb region of the human albumin promoter was obtained from the Dr. Tamaoki from the University of Calgary. Three regions within the 12.5 kb promoter segment have been determined to significantly influence promoter activity (32): 1.) the proximal region comprising the TATA box (550 bp); 2.) an enhancer region at −1.7 kb; and, 3.) a second enhancer region at −6.0 kb FIG. 19. Combined, these regions approximate the strength of the entire 12.5 kb human albumin promoter. The 10.5 kb EcoRI/AvaI fragment of pAlb12.5CAT was co-ligated with the AvaI/HindIII proximal human albumin promoter fragment into the EcoRI/HindIII site of the pBluescript-KS⁺vector, to generate the recombinant plasmid GT4031 (FIG. 20). The 7.2 kb full-length human FVIII cDNA with a 5′ flanking SV40 immediate early intron and a 3′ flanking SV-40 poly-adenylation signal was excised from plasmid GT2051 by XhoI/SalI digestion and w as cloned into the SalI site of GT4031 to generate plasmid GT2053 (FIG. 21). The XhoI fragment derived from plasmid GT2033 containing the minimal ITR region and Ad packaging signal was then cloned into the SalI site of GT2053 in either the forward or reverse orientation to generate the albumin/hFVIII minivirus plasmids GT2059 and GT2061, respectively (FIG. 22). The restriction enzyme digest patterns of the GT2059 and GT2061 minivirus plasmids are shown in FIG. 23.

The FVIII cDNA may be operably linked to a promoter or transcriptional control element that may be synthetic, controllable or regulatable, or tissue/cell type specific. Preferably, expression of the FVIII cDNA in the producer or helper cell is suppressed during viral production and activated following delivery to a target cell. In this manner, differential expression of the reporter or effector gene of the mini-Ad vector is achieved. Such differentiated expression is accomplished by constructing a DNA molecule having the FVIII cDNA under the transcriptional control of a synthetic promoter such as one having a liver-specific enhancer operably linked to an α₁-antitrypsin (α₁-AT) promoter or one in which the tetracycline operon (tetO) is operably linked to the cytomegalovirus (CMV) promoter (tetO-CMV), in which case a cell line is utilized that expresses the tet-KRAB transcriptional repressor protein.

### Example 5

### Homologous Recombination Arms of the FVIII Expression Cassette

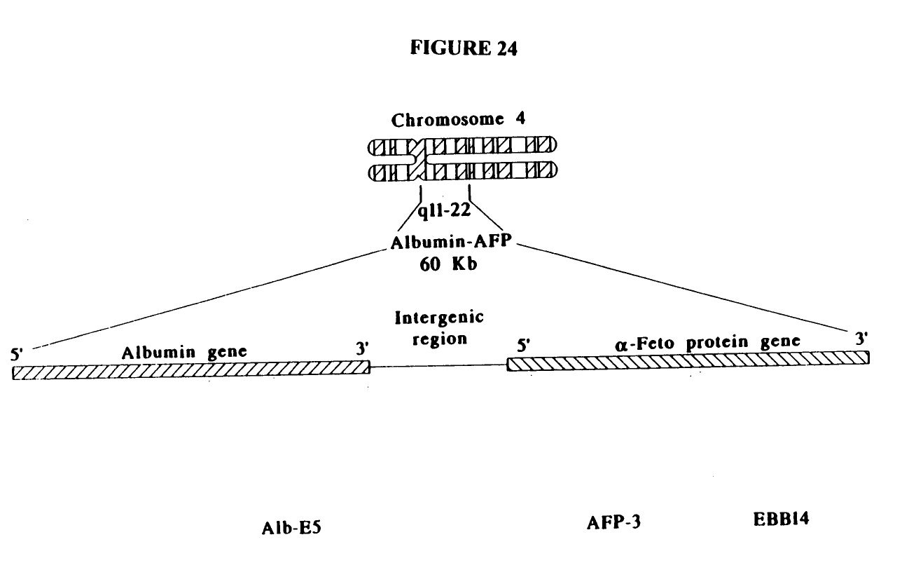

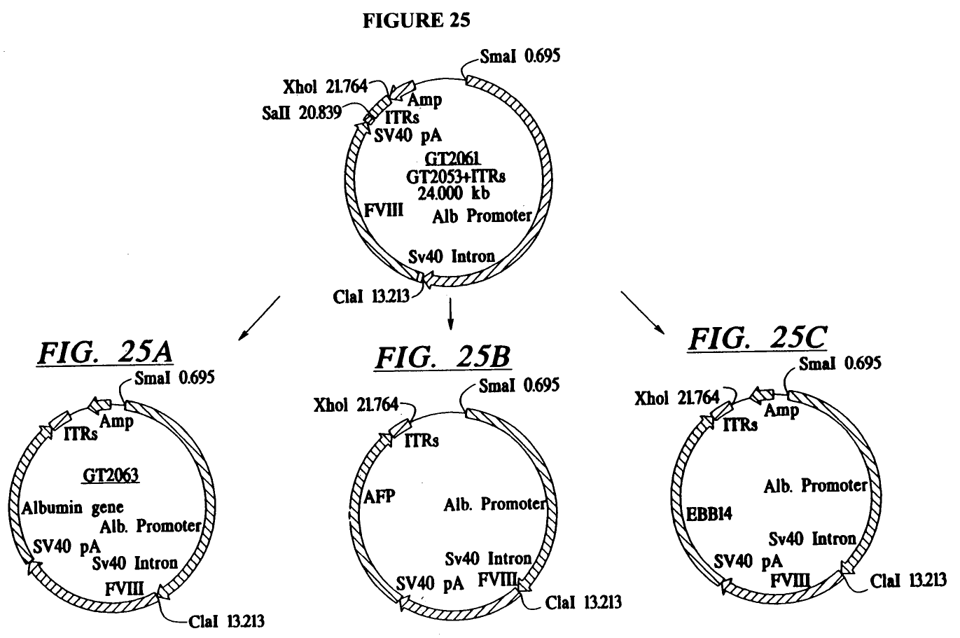

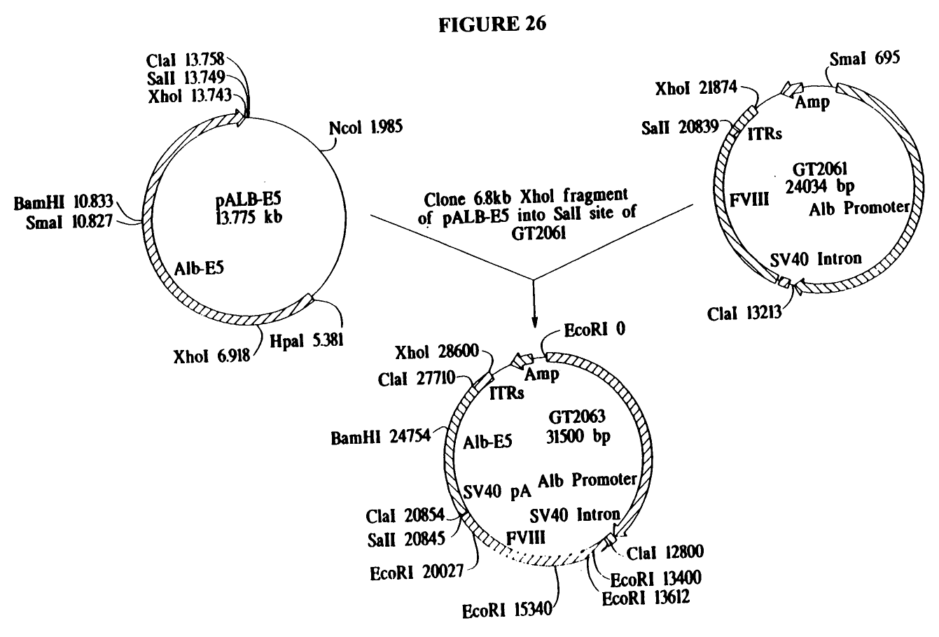

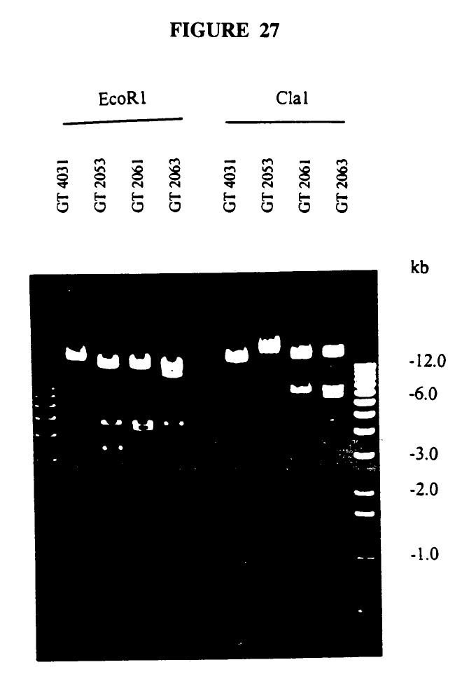

Homologous recombination may be employed to insert an exogenous gene into a the genome of a target cell resulting in stable gene expression. Using this technique, the human FVIII cDNA may be targeted to the genomic DNA of a target cell. Large segments of cellular DNA derived from the human albumin gene or human α-fetoprotein were utilized (32, 33). The 12.5 kb albumin promoter in the FVIII mini-Ad vector functions as the upstream homologous recombination arm while a number of downstream fragments of greater than 6 kb were prepared as potential 3′ recombination arms. A figure of the albumin gene, an intergenic region and the α-fetoprotein gene regions utilized in the present invention is shown in FIG. 24. The structure of the expression cassette in plasmid GT2061 comprising the 12.5 kb albumin promoter at the 5′ end and several regions serving as 3′ homologous recombination arms is presented in FIG. 25. These vectors serve as homologous recombination replacement vectors since the orientation of the arms are in identical orientation as the sequences in the normal human genome. A construct (GT2063) comprising the 3′ XhoI recombination arm derived from the human albumin gene and the pAlb-E5 segment cloned into the unique SalI site of GT2061 is shown in FIG. 25A. Restriction enzyme digestion of the appropriate vectors for construction of the human albumin promoter-driven hFVIII adjacent to a 3′ albumin homologous recombination arm is shown in FIG. 26. Plasmid GT2063 was constructed by insertion of the XhoI albumin gene fragment of plasmid pE5 into the unique Sal I site of GT2061.

### Example 6

### Generation of the FVIII Mini-Ad Vector

The 12.5 kb EcoRI/HindIII human albumin promoter fragment was inserted into pBluescriptKS⁺ (Stratagene, La Jolla, Calif.). The human albumin promoter vector, GT4031, thus contains a unique SalI site into which the human FVIII cDNA (the region in GT2051 from XhoI to SalI comprising the SV40 early intron at the 5′ end and the SV40 polyadenylation signal at the 3 end) was inserted. The resulting plasmid, GT2053, contains unique Sal I and XhoI sites located 3′ to the polyadenylation site (FIG. 21). The Ad minimal ITR and wild type packaging sequence was excised from plasmid GT2033 by XhoI digestion and cloned into the SalI site of plasmid GT2053 to generate plasmid GT2061. The 6.8 kb arm of the albumin gene was isolated from pAlb-E5 and cloned into the unique SalI site of GT2061 to generate plasmid GT2063. GT2063 was transfected into 293 cells together with the helper virus DNA to generate the mini-Ad FVIII minivirus designated GTV2063.

To generate the FVIII minivirus, the helper-virus genome (2 μg) was purified from virus particles and co-transfected with the helper Ad genome (0.2 μg) into 293 cells by calcium phosphate-mediated transfection (81). Following the appearance of CPE, cell-free freeze thaw lysates were prepared and utilized to infect fresh 293 cells. Human FVIII, indicating the presence of the GT2063 mini-Ad, was detected in the cell supernatants using the Coatest FVIII chromogenic assay (Pharmacia). The data are consistent with propagation of a helper/GT2063 mini-Ad vector mixture.

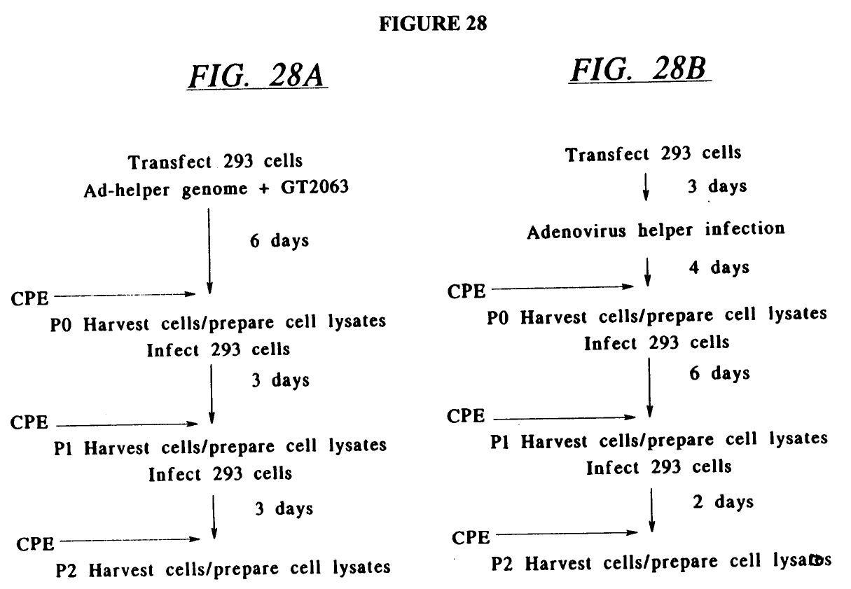

In yet another approach (FIG. 28A), the adenoviral helper plasmid, pBHG10, which lacks the Ad packaging signal and E1 region but encodes the remainder of the Ad proteins, was co-transfected with the mini-Ad clone GT2063 into 293 cells. Rescue of the Ad-minivirus genome was achieved following infection of 293 cells with an E1-substituted helper virus having attenuated packaging function. Both the Ad-helper and mini-Ad genomes may be packaged, and adenoviral particles carrying either genome may be generated using the methodologies of the present invention, although the helper Ad mini-Ad ratios is variable.

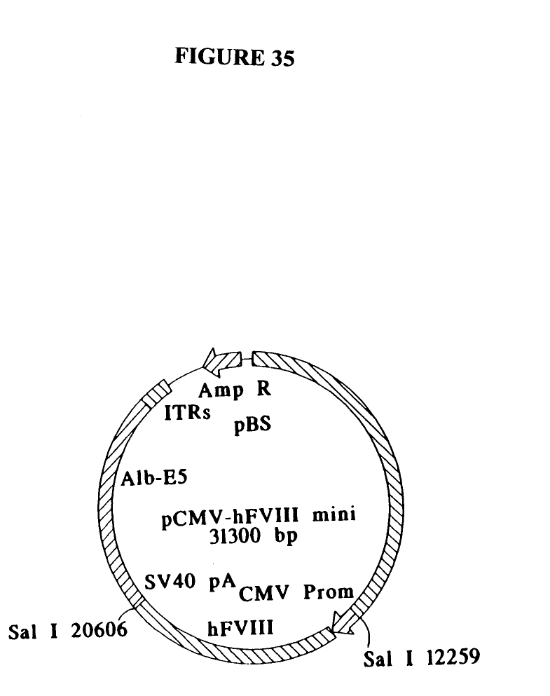

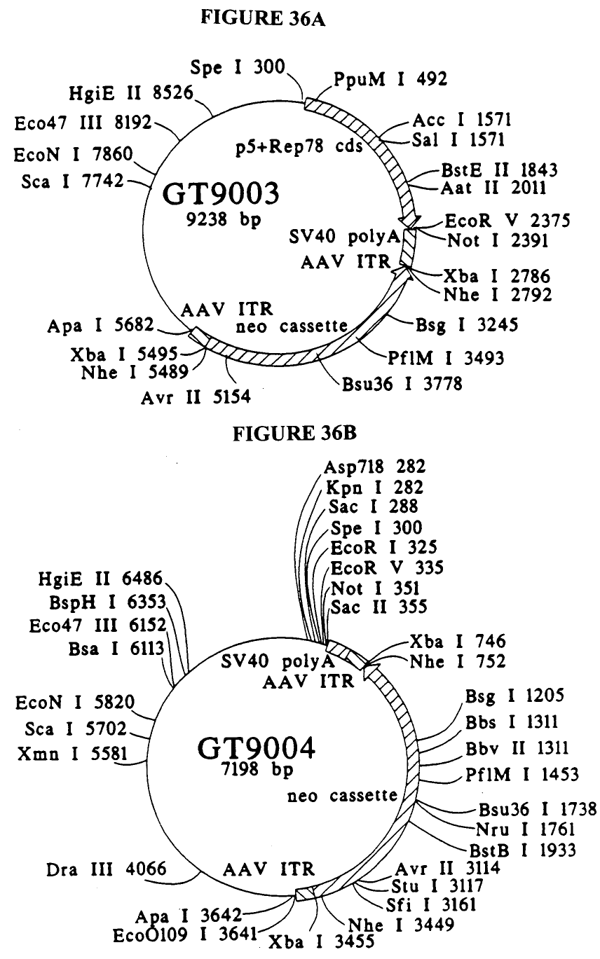

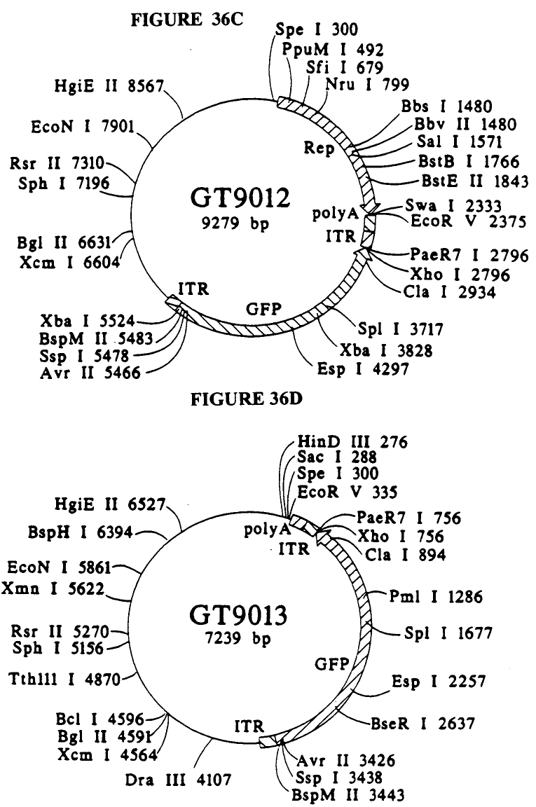

Generation and detection of the FVIII mini-Ad is illustrated in FIGS. 35 and 36, respectively. Helper plasmid pBHG10 (0 μg) and the mini-Ad vector comprising the human FVIII gene (GT2063; 2 μg) were co-transfected into 293 cells by calcium phosphate transfection (81). Transfection into 293 cells may be performed using any of the well-known and widely available techniques such as lipofection (i.e., using Lipofectamine from GIBCO/BRL) or electroporation (i.e., using reagents and electroporator available from Bio-Rad). Infection of the transfected 293 cells with an attenuated helper virus was performed three days after transfection (FIG. 28B). A cytopathic effect (CPE) in FIG. 28B, indicating adenoviral infection has progressed sufficiently, was observed four days after infection. Viral stocks (passage 0 or P0) were then prepared by multiple freeze-thaw of the infected cell pellets. 293 cells were then infected with P0 stocks (1:1) and supernatants collected 24 hours post-infection were positive by PCR specific for the presence of hFVIII sequence. Six days later, a CPE was detected and freeze-thaw lysates (P2) prepared. The P2 lysate was then tested by PCR for the presence of packaged GT2063 mini-Ad and for functional hFVIII. Expression of hFVIII in 293 cells was expected to be minimal because the human albumin promoter is not very active in these cells. This has been determined using both CAT assays (69) and an FVIII chromogenic assay (Helena Laboratories, Pharmacia) following transfection of 293 cells with GT2061 using the calcium phosphate precipitation transfection method.

### Example 7

Amplification and Purification of the Mini-AdFVIII

The applicants have previously filed U.S. patent application Ser. No. 08/658,961 on May 31, 1996 and provided within that application the reagents and methodologies for generating the mini-AdFVIII vector GT2063 and performing the initial passages of propagation (79, 80). In these propagation rounds, the applicants found that the ratio of mini-AdFIII vector to helper virus, AdHβ, increased as propagation progressed. The present application provides an analysis of the vector to helper ratios performed using PCR and Southern blot, both conventional techniques well known to one skilled in the art. For every passage, 500 μl of crude extract of virus (obtained by three freeze/thaw cycles of infected cells) was used to infect a new subconfluent monolayer of 293 cells in a well of a six-well plate. 1 hour after infection 1.5 ml of fresh medium (DMEM/10% FBS) was added. Upon completion of cytophatic effect (CPE), the cells were harvested and the virus extracted again. In each passage, 0.5 ml of the 2 ml were used so the amplification scale of this propagation was 1 to 4. The cleared crude lysate of every passage was used to purify viral DNA by SDS/EDTA/Proteinase K digestion and ethanol precipitation. The viral DNA was used for PCR and Southern blot to detect mini-Ad and, independently, helper Ad virus DNA.

PCR was performed using primers specific to human FVIII cDNA and amplifications were performed on virus subjected to DNAse treatment prior to DNA extraction to remove any residual non-viral contaminating plasmid DNA. PCR was performed using isolated viral DNA as template (1/20 of the viral DNA isolated), FVIII primer #1 at a final concentration of 1 μM (SEQ ID NO:1; ACCAGTCAAAGGGAGAAAGAAGA), FVIII primer #2 at a final concentration of 1 μM (SEQ ID NO:2; CGATGGTTCCTCACAAGAAATGT), and the following conditions: annealing for one minute at 55° C., polymerization for one minute at 72° C., denaturation for one minute at 94° C. for a total of 35 cycles. The results indicated that the FVIII minivirus was present in early passages (passage 3; data not shown).

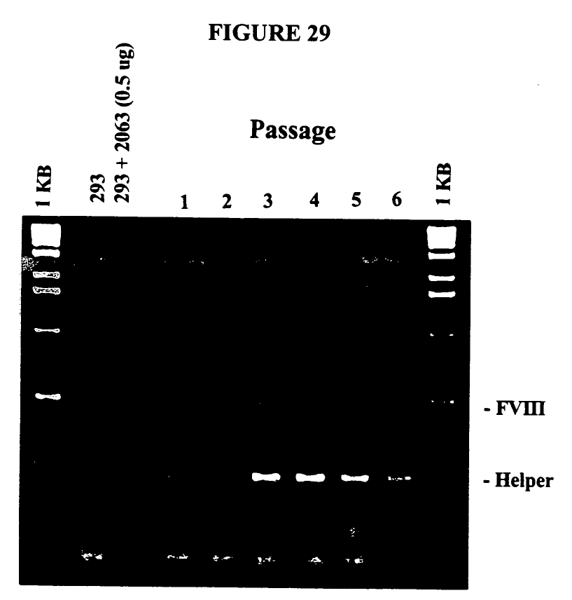

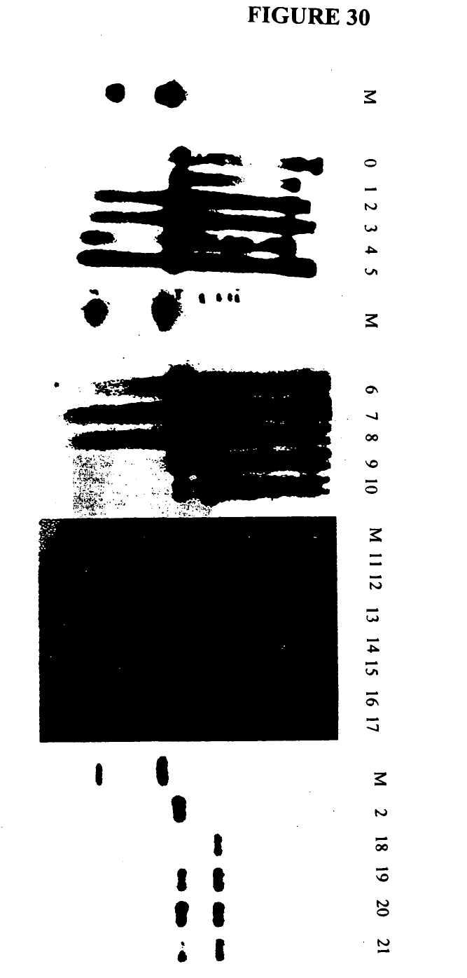

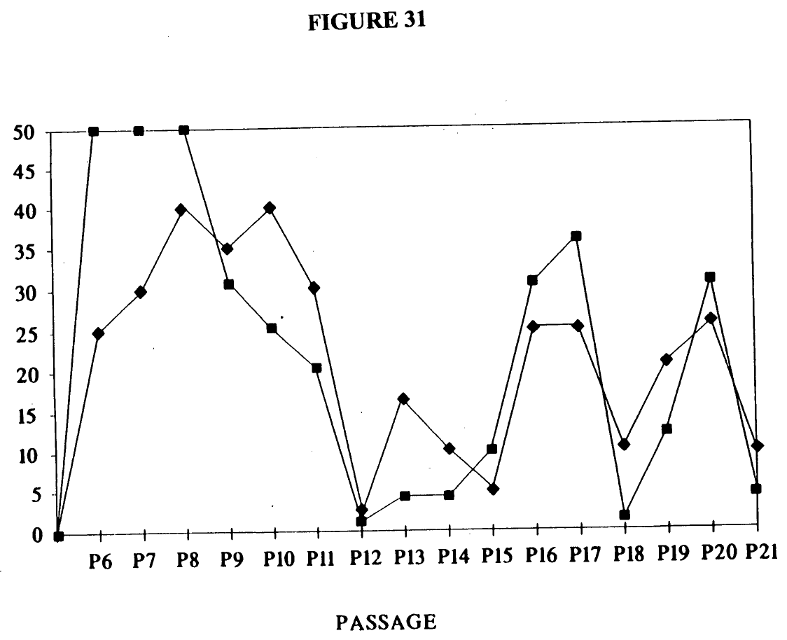

FIG. 29 demonstrates the results of PCR amplification of the packaging signal of the FVIII mini-Ad and, independently, the helper Ad. PCR was also performed on the packaging signal region. PCR was performed using isolated viral DNA (1/20 of the total viral DNA isolated) as template, packaging signal primer #1 (SEQ ID NO:3; GGAACACATGTAAGCGACGG) at a final concentration of 1 μM, packaging signal primer #2 (SEQ ID NO:4; CCATCGATAATAATAAAACGCCAACTTTGACCCG) at a final concentration of 1 μM and the following conditions: annealing for one minute at 55° C., polymerization for one minute at 72° C., denaturation for one minute at 94° C. for a total of 35 cycles. As the packaging signal of the helper Ad is partially deleted, the PCR product from the packaging signal deleted helper is shorter (177 bp) than that of the mini-Ad having a wild-type packaging signal (approximately 310 bp). In the initial passages, the FVIII miniAd was not detected but its presence was increasingly detected in passages 3 to 6. Identical results were obtained using Southern blot analysis (FIG. 30). As a probe in the Southern blot analysis, an Ad DNA fragment adjacent to the right ITR present in both the FVIII mini-Ad and the helper Ad was used. The expected length of the detected fragments after Pst I digestion of the mini-Ad GT2063 and the AdHβ is 3.3 and 2.2 Kb, respectively. FIG. 30 is a compilation of four Southern blots (A-D) of FVIII mini-Ad DNA independently isolated from passages 1 to 21. The 3.3 Kb band corresponding to mini-AdFVIII was detected in DNA isolated from passage 5-21. A steady increase in FVIII mini-Ad DNA was detected until passage 10 which was followed by progressive decrease in FVIII mini-Ad until passage 12. This cycle of increasing and decreasing levels of FVIII mini-Ad DNA was observed to occur approximately every four passages and was accompanied by a parallel cycle of the level of helper Ad DNA, which has a slightly earlier onset. To better define these cycles, the amount of FVIII mini-Ad DNA and helper Ad DNA was quantified densitometrically from the Southern blots and plotted in FIG. 31. The intensity of the bands from equal amounts of marker (1 Kb ladder marker from Gibco, Gaithersburg, Md.) were used to normalize the results of the different blots. The observed cycles match with the well known dynamics of a virus population generated in association with a defective interfering virus (in the system of the present invention, the virus population comprises the FVIII mini-Ad) and a helper virus. The understanding and control of these cycles is important to determine at which passage the mini-Ad vectors should be purified to obtain optimal titers. Passages such as #18 (P18) result in a vector preparation enriched for the FVIII mini-Ad (i.e., P18 appears to contain 10 times more FVIII mini-Ad than helper Ad), albeit at a low titer. Passages such as #20 (p20), comprise high levels of FVIII mini-Ad and helper Ad, although at an undesirable FVIII mini-Ad to helper Ad ratio of 1:1.

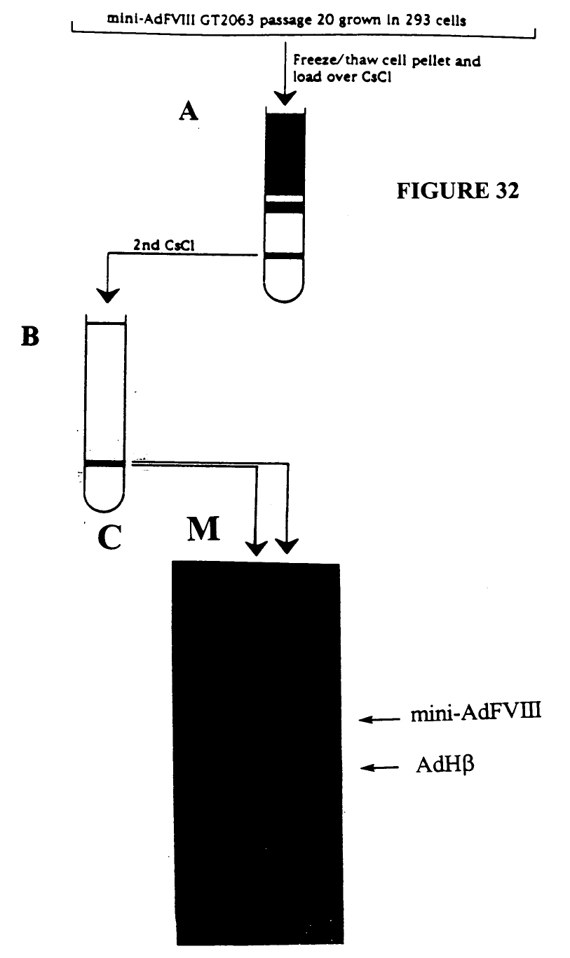

A large scale amplification was performed at p20. One hundred 15-cm dishes, each comprising approximately approximate 109 infected 293 cells (ATCC# CRL1573) were harvested upon completion of the CPE. A crude lysate was then prepared by three freeze thaw cycles to extract the virus. The crude lysate was cleared by centrifugation, loaded onto a step density gradient of CsCl (three layers of 1.5, 1.35, and 1.25 g/ml) and centrifuged at 35000×g for 1 h. The band corresponding to the mixture of mini-Ad and helper Ad was further purified using a second continuous CsCl gradient of 1.35 g/ml. After 16 h centrifugation at 35,000 rpm (150,000×g), two bands of similar intensity were observed, isolated separately, and dialyzed into phosphate buffered saline (PBS) containing 10% glycerol. Viral DNA was purified from an aliquot of each band and the amounts of mini-AdFVIII and Helper analyzed by Southern blot (FIG. 32). The upper band (lighter) appeared to comprise mainly mini-Ad and the lower band mainly helper Ad. These results were expected as the FVIII mini-Ad genome (GT2063=31 Kb) is smaller than the helper Ad genome (AdHβ=37.1 Kb), consistent with previous CsCl fractionation results for the M32 mini-Ad demonstrated in the parent application (U.S. Ser. No. 08/659,961 filed on May 31, 1996) of the present application.

### Example 8

### Test of the Mini-AdFVIII in Cell Lines

The FVIII mini-Ad (GT2063) was purified by CsCl as described above and utilized to demonstrate production of FVIII in host cells infected with the vector. To this end, 293 and HepG2 cells were utilized due to their known ability to utilize the albumin promoter. FVIII production in these cells was assayed by immunohistochemistry and functional assays 24 h after infection. Purified FVIII mini-Ad vector was added to 0.5 ml of medium and used to infect 6×10⁵ 293 and HepG2 cells in a 4 cm² well. After a 4 h incubation to allow for adsorption of the viral particles to the host cells, the infection medium was replaced with fresh medium. Following infection of 293 cells with 0.1 μl of a 1/100 dilution of the lighter fraction comprising the FVIII mini-Ad and immunohistochemical analysis of the cells for the presence of human FVIII, approximately 10% of the cells stained positive for FVIII expression.

293 cells were grown in chamber slides and infected with a diluted (1/100) 1 μl aliquot of the upper or lower fractions as shown in FIG. 18C. Twenty-four hours following infection, the cells were fixed and stained with a FVIII specific mAb (Cedar Lane Sheep anti-human FVIIIC, #CL20035A, Accurate Chemical and Scientific Corporation, Westbury, N.Y.) and subsequently a secondary antibody (biotinylated donkey anti-sheep IgG. Jackson Immunoresearch, #713-065-147) and DAB (resulting in a reddish-brown color. SIGMA Cat. No. D7679). A. Mock, uninfected control 293 cells. Transduction with two independent preparations of the upper fraction as shown in FIG. 32 (mini-Ad FVIII enriched). Ten percent of the cells stained positively for FVIII expression. Transduction with the lower fraction as shown in FIG. 32 (helper virus enriched) resulted in 0.1% of the cells staining positive for FVIII expression.

The estimated titer in transducing units per milliliter was determined to be 6×10⁹ transducing units/ml. If an adsorption time of 4 h, an adsorption volume of 0.5 ml in 4 cm², and a non-rocking adsorption are taken into account, the estimated titer may be reduced by a factor of 0.42, 0.56, and 0.53, respectively (49). The actual titer of mini-AdFVIII vector would therefore be estimated to be 4.6×10¹⁰ transducing unit/ml. The titer determined by optical absorbance at 260 nm, which reflects the number of viral particles per milliliter was determined to be 3.6×10¹² particles/ml for the lighter fraction of FVIII mini-Ad. Therefore, the bioactivity of the FVIII mini-Ad can be calculated to be one FVIII-transducing unit per every 78 viral particles, which falls within the levels of acceptability recommended by the Food and Drug Administration (49).

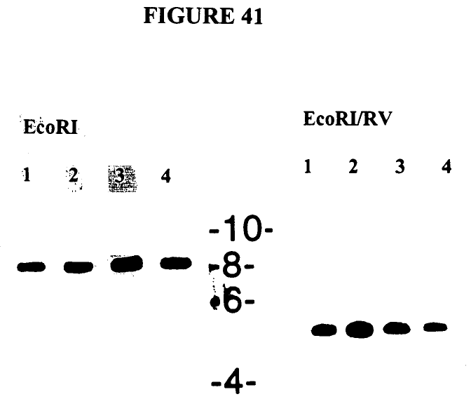

The amount of functional FVIII in the supernatant of transduced cells was determined using the chromogenic Coatest FVIII Test (Pharmacia Piscataway, N.J.). A Coatest chromogenic assay for functional FVIII was performed. A standard curve in triplicate from 4000 ng/ml to 62.5 ng/ml (rows A to G) was plotted to obtain the equation to extrapolate the readings from the samples. Experiments were performed in triplicates. 10 μl aliquots of miniAdFVIII in 293 cells; 1 μl aliquots of mini-AdFVIII in 293 cells; 10 μl aliquots of mini-AdFVIII in HepG 2 cells; 1 μl aliquots of miniAdFVIII in HepG 2 cells; conditioned medium from untransduced 293 cells; and conditioned medium from untransduced HepG 2 cells were independently tested. One million 293 cells or, independently, HepG2 cells, were infected with an excess amount of purified vector in order to achieve 100% transduction. Infection conditions were as described above and the supernatants (2 ml) were collected 24 h after infection. To generate a standard curve, a standard human plasma sample was serially diluted in cell culture medium to obtain a final FVIII concentration range from 62.5 to 4000 ng/ml. The results are shown in FIG. 41. The amount of FVIII detected in HepG2 and 293 supernatants were 0.8 and 0.23 ng/ml respectively. Therefore, the total amount of FVIII produced in 24 h was 1.6 ng per million HepG2 cells and 0.46 ng per million 293 cells.

To further analyze the structure of the hFVIII protein produced by MiniAdFVIII, HepG2 cells were infected with the vector and the conditioned medium was used for immunoblot analysis. Detection with a specific antibody to the heavy chain revealed several protein species ranging in size from 100 kDa to 200 kDa. A unique band of 80 kDa was detected with specific antibodies to the light chain. The pattern obtained from infected cells was comparable to that of recombinant FVIII (Hyland-Immuno, Baxter Healthcare Corp.). An observed slight difference in the intensity of the bands of the heavy chain may reflect a difference in post-translational processing in different cell lines, since the recombinant FVIII was produced from CHO cells. Taken together, the results demonstrate that hFVIII produced in vitro by the MiniAdFVIII-infected cells is biologically active and has the expected protein structure.

### Example 9

### In Vivo Utilization of the Mini-Ad FVIII Vector

A. Methods

MiniAdFVIII activity studies. Six to eight week old C57BL/6 and Balb/c mice were purchased from Harlan. Hemophilic mice (exon 16-disrupted FVIII knock-out mice) were obtained from the University of Pennsylvania²⁸ and bred in house. For dose-response studies, groups of three mice were injected via tail vein with different doses of MiniAdFVIII. At the designated timepoints, blood was collected by retro-orbital puncture in 0.1 volume of 0.1M sodium citrate. Cells were removed by centrifugation and the plasma was tested for hFVIII by ELISA or functional assay.

Phenotypic correction studies. Groups of 12 mice were injected on day 1 with 2.5×10¹¹ vp of MiniAdFVIII or vehicle (PBS), respectively. On day 3, blood was collected by retro-orbital puncture with a glass capillary tube. Tubes were broken at one minute intervals to check for clot formation and the clotting time was recorded. On day 6, a 2-cm section of the tail was clipped from each mouse to measure the bleeding time (time until bleeding stopped) and the volume of blood shed.

Toxicity studies. Groups of six C57BL/6 mice were injected via tail vein with the selected doses of MiniAdFVIII or with vehicle. Three and 14 days after injection, 3 mice from each group were sacrificed. Blood was collected by cardiac puncture and serum samples were tested for ALT levels (Boehringer Manheim). Tissues (liver, spleen, kidney, lung and heart) were fixed in formalin for 24 hr, paraffin-embedded and processed for histopathology.

Analysis of Vector DNA in vivo. PCR assays with hFVIII specific primers (FIG. 6A) were used for detecting DNA of the MiniAdFVIII and ancillary vectors. Liver tissue (0.25 g per sample) from MiniAdFVIII-treated mice was ground to a powder in a liquid nitrogen-cooled mortar and DNA was extracted using a Qiagen Tissue Kit (Qiagen Inc., Valencia, Calif.). Each PCR reaction contained 1× reaction buffer, 200 μM each deoxynucleotide triphosphate, 2.25-4.0 Mg⁺⁺ (the concentration varied with different primers), 0.4 μM of each primer and 2.5 units of Qiagen HotStar Taq polymerase in a total volume of 50 μl. Amplification was carried out for one cycle of 15 min at 95° C. followed by 35 cycles of 30 sec at 94° C., 30 sec at 52° C. and 45 sec at 72° C. with a final extension of 7 min at 72° C. Aliquots of the PCR reactions (15 μl) were analyzed on 1.5% agarose gels. Amplified DNA fragments were semi-quantitated by comparing Gelstar-stained band intensities from tissue samples to those for the control plasmid (Gelstar, FMC BioProducts, Rockland, Me.). Division of the copies by pg of input DNA yielded copies/pg input DNA. These values were normalized to Copies/Cell Equivalent by assuming 6 pg/cell nucleus then adjusting values accordingly.

Analysis of anti-hFVIII and anti-MiniAdFVIII antibodies. ELISA assays were used to determine the antibodies against the hFVIII protein and the MiniAdFVIII vector. The assay plates, either coated with purified hFVIII protein or the ancillary vector in carbonate buffer, were blocked with PBS containing 4% FBS for 1 hour at room temperature, washed once, and incubated overnight with serially diluted test plasma samples (100 μl/well). After 5 washes, each well was incubated with 1001 μl of 1:2000 diluted goat anti-mouse IgG (H+L) conjugated to HRP (Southern Biotechnology Associates) at 37° C. for 1 h. After 5 washes, 100 μl of peroxidase substrate solution (o-Phenylenediamine) was added to each well. Color development was monitored for 7 min, and the reactions were stopped with addition of 3N HCl (50 μl/well). The optical density at 490 nm (OD₄₉₀) was read using an EL340 spectrophotometer (Bio-tek Instruments, Winooski, Vt.).

B. Safety Studies of MiniAdFVIII Vector and Ancillary Ad

Safety studies of MiniAdFVIII were carried out in mouse and dog models. Three different doses (3×10′ vp, 1×10¹¹ vp and 2.5×10¹⁰ vp) around the therapeutic range were evaluated for histopathology of heart, lung, liver, spleen, and kidney tissues, 3 and 14 days post-injection. Other markers of liver injury such as liver-specific transaminase levels in serum, or liver apoptosis and regeneration were also studied. A summary of the histopathology analysis is shown in Table 2.

TABLE 2
Summary of Histopathology Studies

| | Sampling Time | | | | | | | |
|---|---|---|---|---|---|---|---|---|
| | 3 Days After Injection | | | | 14 Days After Injection | | | |
| | Test Group | | | | | | | |
| | A | B | C | D | A | B | C | D |
| Heart | N | N | N | N | N | N | N | N |
| Lung | N | N | N | N | N | N | N | N |
| Liver | 0.25 | 0.17 | 0.08 | 0.25 | 0.5 | 0.25 | 0.25 | 0.25 |
| Inflammation/ | | | | | | | | |
| Necrosis | | | | | | | | |
| Spleen | 2.0 | 2.0 | 1.67 | 0 | 2.0 | 2.0 | 0.33 | 0 |
| Extramedullary | | | | | | | | |
| hematopoiesis | | | | | | | | |
| Spleen | 0.5 | 0.5 | 0.5 | 0.25 | 0.5 | 0.5 | 0.42 | 0 |
| Apoptosis | | | | | | | | |
| Kidney | 0.75 | 0.5 | 1.33 | 0.63 | 0.88 | 0.58 | 0.67 | 0 |
| Degeneration/ | | | | | | | | |
| Necrosis | | | | | | | | |
| Kidney | 1.00 | 0 | 0 | 0 | 0 | 0 | 0.08 | 0 |
| Inflammation | | | | | | | | |

Severity grading: 1 = minimal; 2 = mild; 3 = moderate; 4 = marked. The results are the average number for all animals examined with a correction factor for lestion appearance (focal, multifocal, or locally extensive) N = no change

As shown in Table 2, no microscopic changes were observed in the heart and lungs of test animals. Other minimal changes in the liver and kidneys of both experimental and control groups were regarded as unrelated to the administration of MiniAdFVIII. A mild-grade increased extramedullary hematopoeisis was found in all the injected mice irrespective of the virus dose. A characteristic feature was the presence of clusters of proliferating cells in the spleen. There was no dose-dependent ALT increase at any timepoint, indicating that injection of the highest dose did not cause significant liver injury. Other markers of liver injury were studied, such as hepatocyte apoptosis and expression of proliferating cell nuclear antigen (PCNA), which reflects the proliferating status of the liver. In both cases, liver sections for the highest dose group revealed similar numbers of apoptotic and proliferating cells as the control group for both timepoints in accordance with the ALT and histopathology data. Biodistribution of MiniAdFVIII determined by PCR analysis on the tissue samples revealed that the injected vector was mainly distributed to the liver with lesser amounts in spleen, kidney, and lung. The vector DNA was not detectable in other organs and tissues, particularly not in testis.

In hemophilic dog models, two dogs at 4 weeks of age were given 1.1×10² and 2.6×10¹² vp/dog intravenously. A third dog was given the same volume of vehicle (10% glycerol in PBS) as a negative control. At various time points prior to injection and from day 1 up to day 85 postinfection, general conditions of the treated animals were recorded and blood samples were collected for monitoring levels of hFVIII expression and liver enzyme (ALT). No differences were observed in body weight and other physical conditions among the treated dogs and the control dog. The ALT was at a range from 20-50 mg/ml in the treated animals and had no difference from the measurements performed on the blood samples prior to the injection. In this study hFVIII expression was not detected. One possible reason for this is that the human albumin promoter is not fully active in dogs or the doses given were not high enough, which reflects that there may be a threshold dose level for a given vector to generate detectable amount of transgene product. The MiniAdFVIII-treated dogs were sacrificed on days 57 and 85 postinjection and tissue samples were collected. PCR analyses demonstrated that vector DNA (hFVIII sequence) was detectable in liver samples, indicating that gene delivery was successful.

To determine the potential risk of contamination of the ancillary Ad in the MiniAdFVIII preparation, a toxicity study with the ancillary Ad alone was carried out in Balb/c mice. Five dose groups with 5 mice per group were given tail vein injections of the ancillary Ad at 5×10⁷, 5×10⁸, 5×10⁹, 5×10¹⁰, and 4×10¹¹ vp/mouse respectively. Vehicle (PBS) was used as a negative control. From days 1-14 postinjection, no differences in general condition and body weight were observed between the treated mice and the PBS control. On day 14 postinjection, the mice were sacrificed. Tissue (liver, spleen, heart, lung, and kidney) samples were collected and analyzed for histopathology changes. Again, no difference was detected in the treated mice compared to those with the PBS control. These data indicate that the potential clinical risk of MiniAdFVIII is very low and there is a significantly large margin of safety for the ancillary Ad.

To further evaluate the safety of the vector in mice, an acute toxicity study was performed in C57BL/6 mice via systemic administration of MiniAdFVIII. Three groups of mice were injected with a single dose of the vector at 3×10¹¹ vp, 1×10¹¹ vp, or 2.5×10¹⁰ vp per mouse. Tissue samples of heart, lung, liver, spleen, and kidney were collected on days 3 and 14 p.i. for histopathology analysis. A summary of the analysis is shown in Table 3. No microscopic changes were observed in the heart or lung samples of the mice. Other minimal changes (scoring 1) in the liver and kidneys of both groups were regarded as unrelated to the administration of MiniAdFVIII. A mild-grade extramedullary hematopoeisis characterized by the presence of clusters of proliferating cells in the spleen was found in all the injected mice irrespective of the vector dose used.

Levels of serum alanine aminotransferase (ALT), a specific liver injury marker, were determined and compared among the test animals. No dose-dependent ALT increase was detected at any timepoint, indicating that injection of the highest dose did not cause significant liver injury (data not shown). Other markers of liver injury were studied such as hepatocyte apoptosis and expression of proliferating cell nuclear antigen (PCNA) which reflects the proliferating status of the liver. In both analyses, liver sections for the highest dose group revealed similar numbers of apoptotic and proliferating cells as were found in the control group for both timepoints (data not shown).

C. Studies of MiniAdFVIII Activity in Mice

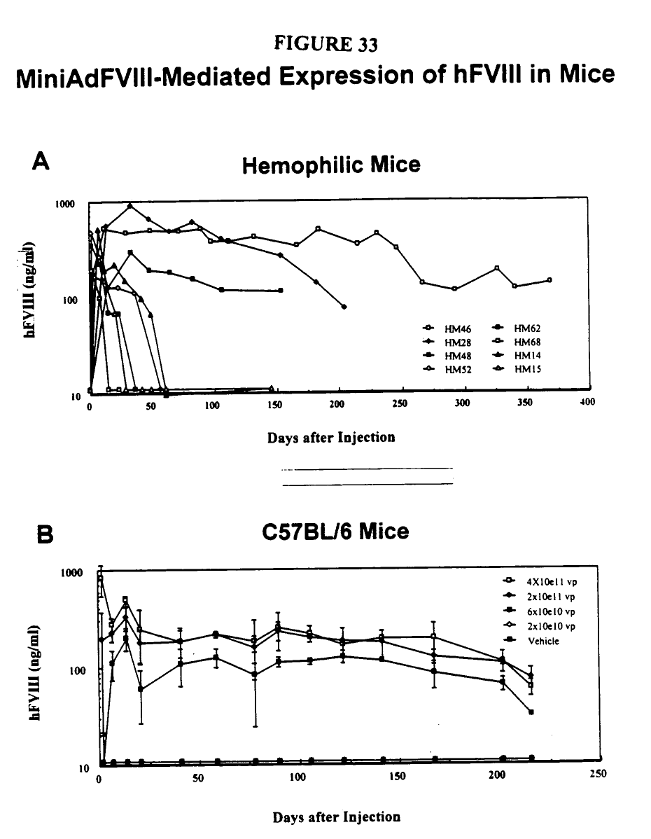

To assess the gene transduction efficiency of MiniAdFVIII and the subsequent hFVIII production in vivo, a dose-response study of the vector was performed in C57BL/6 mice (FIG. 33B). Groups of three mice were injected via tail vein with different doses of MiniAdFVIII. At various timepoints, plasma was collected and assayed for hFVIII expression by ELISA. Doses of 4×10¹¹ and 2×10¹¹ vp MiniAdFVIII mediated hFVIII expression in mice at levels above the human physiological levels (>200 ng/ml). Accordingly, 6×10¹⁰ vp produced relatively lower levels of hFVIII (˜100 ng/ml) and at 2×10¹ vp hFVIII levels were below the assay detection limit. The expression of hFVIII in the mice persisted for 308 days (last timepoint tested) although a gradual decline in hFVIII expression was observed after day 160 post-injection (p.i.). A dose-response study in Balb/c mice revealed that the same doses yielded initial levels of hFVIII similar to those in C57BL/6. However, expression levels declined sharply after day 14 p.i.

In another experiment, hemophilic mice (Bi, et al. Targeted disruption of the mouse factor VIII gene produces a model of haemophilia A. Nat. Genet. 10:119-121, 1995) were injected with a single dose (2×10¹¹ vp) of MiniAdFVIII and monitored for expression of hFVIII by ELISA and/or a Coatest chromogenic assay (FIG. 33A). Even though all of the mice were shown to express hFVIII at human physiological levels following administration, the persistence of hFVIII expression varied greatly among individuals. In 3 out of 16 mice injected, hFVIII expression could be detected at all timepoints tested ranging from 5 months through over one year; in the remaining 13, hFVIII levels decreased to undetectable levels 3-8 weeks after injection.

A phenotypic correction study was carried out to evaluate whether the MiniAdFVIII-mediated expression of hFVIII was able to correct the deficient hemostasis in hemophilic mice. Two groups of 12 hemophilic mice were injected with either 2.5×10¹¹ vp MiniAdFVIII or vehicle (PBS), respectively. A clotting test assay was performed on day 3 and a tail clipping experiment was carried out on day 6 p.i. to measure the bleeding time and blood loss. As a control, the same tests were performed in 10 untreated C57BL/6 mice. All of the MiniAdFVIII-treated mice expressed the hFVIII (104±26 ng/ml) as measured by a Coatest functional assay. Clotting time was reduced from 5.3±1.87 min in the vehicle-injected mice to 2.58±0.51 min in the MiniAdFIII-treated mice (p<0.002). The clotting time in the MiniAdFVIII-treated mice was shorter than that (3.40±0.69 min) in C57BL/6 controls.

Upon tail clipping, bleeding times in the MiniAdFVII-vector-treated hemophilic mouse group were significantly improved (˜30 min) compared to the continuous bleeding with no survival in the vehicle-treated hemophilic mice and the blood flow rate was reduced from 20.9±4.39 μl/min to 8.6±1.07 μl/min. This compares favorably to the blood flow rate of 4.8 μl/min in C57BL/6 controls. Although the bleeding phenotype was corrected by the hFVIII, in contrast to the result obtained using vehicle-treated control mice, the clotting time was about 3 times that of normal C57BL/6. The reason behind this phenomena is to be understood by further studies. Altogether, these results indicate that the hFVIII expressed from MiniAdFVIII in vivo was functional and able to correct the bleeding phenotype of hemophilic mice.

D. Assessment of MiniAdFVIII Immunogenicity in Mice

To understand the immunogenicity of MiniAdFVIII and the nature of the variations observed in the duration of hFVIII expression in the MiniAdFVIII-treated mice, anti-hFVIII as well as anti-Ad antibodies were analyzed by ELISA. Those mice with the shortest duration of hFVIII expression had humoral immune responses to the transgene product. In all cases, the rise in antibody titer inversely correlated with the level and duration of hFVIII expression. Administration of MiniAdFVIII induced a stronger and faster humoral immune response in Balb/c mice. In these mice the titers of the anti-hFVIII antibodies rose sharply within a week after injection of the vector (data not shown).

Although the C57BL/6 strain was previously shown not to produce anti-hFVIII antibodies upon tail vein adenovirus delivery (Connelly, et al. Sustained expression of therapeutic levels of human factor VIII in mice. Blood 87:4671-4677, 1996), a low titer of anti-hFVIII antibodies was detected in the plasma of these mice starting around day 122 p.i. This low titer may be sufficient to account for the observed decline in hFVIII expression (data not shown).

Analyses of the anti-Ad antibodies indicated that all of the MiniAdFVIII-treated mice generated humoral immune responses to the adenoviral proteins, regardless of the strain. However, the levels and duration of the antibodies in individual mice varied significantly. In contrast, the presence of anti-Ad antibodies did not affect the persistence of the hFVIII expression, even in the hemophilic mice that had long-term gene expression of hFVIII. The anti-viral antibodies gradually declined to low levels after three months post injection. Taken together, the in vivo data indicate that in the absence of an antibody response to the transgene product, MiniAdFVIII is able to mediate a long-term expression of hFVIII in mice at human physiological levels. Humoral immune responses to the MiniAdFVIII vector were detectable, but apparently did not affect the level or duration of hFVIII expression in either C57BL/6 or hemophilic mice.

E. Characterization of hFVIII Produced In Vivo

The hFVIII protein secreted into the plasma of injected mice was analyzed by immunoprecipitation and western blotting using specific antibodies to hFVIII. As in the in vitro assay, both heavy and light chains were detected with molecular masses of 200 kDa and 80 kDa, respectively, indicating correct post-transcriptional processing of the human protein in the murine host.

In order to assess the integrity and functional properties of the hFVIII expressed in the mouse model, the hFVIII produced from the vector was compared to the recombinant hFVIII protein in plasma samples of the treated hemophilic mice. The plasma samples were collected either on days 3 and 6 p.i. of MiniAdFVIII (2.5×10¹¹ vp/mouse) or at 1.5 hr post-injection of the recombinant protein (3.5 or 14 units/mouse). Specific activities of the proteins derived from the two sources were calculated by dividing the values derived from the chromogenic assay for FVIII function by those derived by the ELISA assay for FVIII protein. The results indicate that the specific activities of the transgene product and the recombinant hFVIII in mouse models are comparable. The relatively low activities of the protein detected in the vector-treated mice (both days 3 and 6 post-injection) and the high-dose group of recombinant hFVIII-treated mice need to be further studied. However, the specific activities of the hFVIII protein generated from the MiniAdFVIII-infected cells in vitro were within the same range as that of recombinant hFVIII at above 4 to 8 Units/[g protein.

F. Biodistribution and Status of MiniAdFVIII DNA In Vivo

PCR analysis was used to detect biodistribution of the vector following the systemic administration of MiniAdFVIII. The vector DNA was detectable in the tissue samples from liver, spleen, lungs, kidneys, and heart of the treated mice (data not shown), with the majority detected in the liver, as previously reported (Vrancken Peeters et al. Method for multiple portal vein infusions in mice: quantitation of adenovirus-mediated hepatic gene transfer. BioTechniques 20:278-285, 1996; Huard, et al. The route of administration is a major determinant of the transduction efficiency of rat tissues by adenoviral recombinants. Gene Ther. 2:107-115, 1995; Li, et al. Assessment of recombinant adenoviral vectors for hepatic gene therapy. Hum. Gene Ther. 4:403-409, 1993). No signal was detected in gonadal (testis) tissue with the limit of sensitivity of the PCR assay being 1 copy per 1 μg tissue.

The sustained expression of hFVIII indicates the presence and activity of the vector DNA in treated animals. To analyze the DNA status of MiniAdFVIII, a set of five pairs of PCR primers were designed to detect different segments along the MiniAdFVIII genome, as shown in Table 3.

TABLE 3
Primers Used to Detect MiniAd FVIII DNA

| | | |
|---|---|---|
| Primers | Positions | Sequences |

| | | |
|---|---|---|
| A-forward | 0.135-0.154 | GGAACACATGTAAGCGACGC (SEQ ID NO. 12) |
| A-reverse | 0.516-0.535 | GGTGCTCTTCTGATTATGGA (SEQ ID NO. 13) |
| B-forward | 7.620-7.640 | GTATCTTATCATGTCTGGATC (SEQ ID NO. 14) |
| B-reverse | 7.880-7.898 | CACCGTTACTGACTCGCTA (SEQ ID NO. 15) |
| C-forward | 14.841-14.858 | CTATTTGCATGGCTGGCG (SEQ ID NO. 16) |
| C-reverse | 15.542-15.559 | CATGCGTGAGTACTTGTG (SEQ ID NO. 17) |
| D-forward | 27.609-27.629 | AGGATATACACTAGGCTTAAG (SEQ ID NO. 18) |
| D-reverse | 28.868-28.887 | GACACGGAAATGTTGAATAC (SEQ ID NO. 19) |
| E-forward | 30.372-30.390 | AATACGCAAACCGCCTCTC (SEQ ID NO. 20) |
| E-reverse | 30.751-30.767 | CCGTGTCGAGTGGTGTT (SEQ ID NO. 21) |

The three treated mice with long-term transgene expression (HM48, HM28, and HM46) were sacrificed and liver DNA was extracted for the PCR assays. Specific PCR products of the predicted sizes were obtained and the copy numbers of the representative fragments were quantified on a cell equivalent basis. Although the copy numbers varied between individual mice, an approximate equal number of vector copies/cell were detected within each mouse, regardless of the fragment tested. Thus, the complete vector genome persisted in the transduced cells without apparent loss or amplification of any specific portion. In contrast, no specific products were detected for either of the two control mice (data not shown).

These results show that the MiniAdFVIII vector was mainly distributed in liver via i.v. administration and the vector DNA stayed likely in an episomal form in the mouse liver.

### Example 10

### Improvement of the Mini-AdFVIII In Vivo

Improvements in the vector system were accomplished by generation of a vector into which various expression cassettes may be cloned. The vector GT2063 was modified by excising the proximal albumin promoter region and human FVIII gene localized between the Pme I and Sal I sites. This was accomplished by first converting the Pme I site of GT2063 to a Sal I site by ligating a SalI linker to the Pme site. The resulting clone, GT2072, was treated with Sal I and religated to remove the proximal albumin promoter/hFVIII gene region thereby creating a mini-Ad vector having a unique Sal I cloning site for insertion of various expression cassettes. The expression from such cassettes may be affected by albumin gene enhancers located upstream. Each clone was analyzed to determine the level of expression of the transgene.

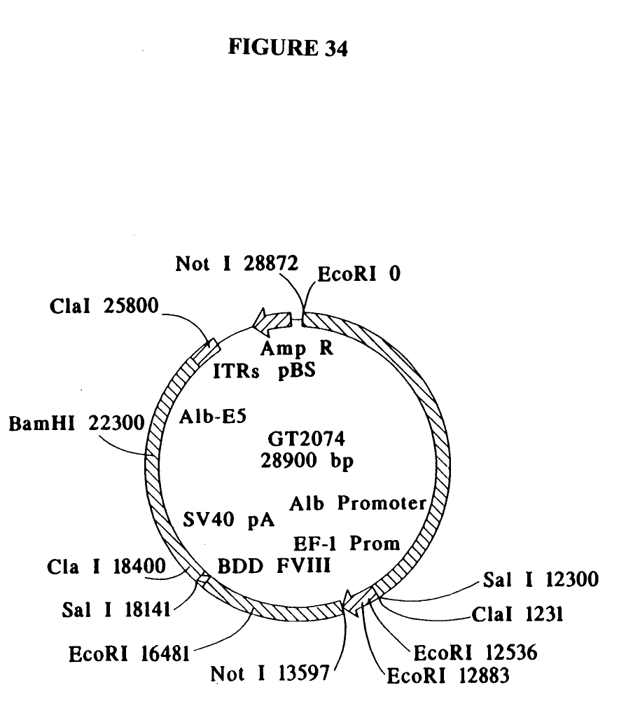

Expression cassettes were prepared for insertion into the improved vector, GT2072. The expression cassettes of this example comprises the cytomegalovirus (CMV) immediate early promoter, the elongation factor I (EF-I) promoter (which are known to function in a wide variety of cell types) or the liver-specific promoter for the phosphoenol pyruvate carboxykinase (PEPCK) gene. The EF-I and CMV promoters were each separately utilized to drive expression of either the full-length FVIII cDNA or the B-domain deleted (BDD) factor VIII cDNA (FIGS. 34 and 35, respectively). An EF-1 BDD FVIII cassette flanked by Sal I sites was then cloned into the Sal I site of GT2073 resulting in generation of the plasmid illustrated in FIG. 34. An expression vector comprising the full length human FVIII coding region under control of the CMV promoter was also constructed and is illustrated in FIG. 35.

### Example 11

### Construction of an Integratable AAV-ITR/Rep System-Based Vector

Adeno-associated virus is a human non-pathogenic single-stranded linear parvovirus that replicates only in the presence of a helper virus like adenovirus or herpes virus. However, in the absence of helper, AAV can integrate specifically in the host genome and be maintained as a latent provirus (34). The particular locus where AAV integrates has been located to chromosome 19q13.3-qter and named AAVS1 (22-25, 35).

The mechanism of AAV integration has not been fully elucidated. However, two viral elements have been implicated in this process: the AAV ITRs and two forms of the Rep viral proteins (Rep78 and Rep68). The AAV ITRs (Inverted Terminal Repeats) are palindromic sequences present in both ends of the AAV genome, that fold into hairpin structures and function as origins of replication. Several activities have been described for Rep78/68 proteins including sequence-specific DNA binding (36, 37), sequence and strand-specific endonuclease activity (38), and ATP-dependent helicase activity (38-40). These proteins can bind to a specific sequence in the ITR DNA and promote the process named terminal resolution by which the ITR hairpin is nicked and replicated. A Rep-binding motif and a terminal resolution site (trs) have been identified in both the AAV ITR and AAVS1 and demonstrated to promote in vitro DNA replication in the presence of Rep (28). It has also been shown that Rep68 protein can mediate complex formation between the AAV ITR DNA and AAVS1 site in vitro (41). These findings suggest a model in which the DNA binding and endonuclease activity of Rep along with limited DNA synthesis at the ITRs and AAVS1 sites would allow targeted integration of the sequences contained between the ITRs (27).

AAV has been considered as a candidate vector for gene therapy. However, the limited size of exogenous DNA that it can accept (4.2 Kb), the difficulty in getting high titers in large-scale preparations, and the loss of specific integration of the recombinant AAV have posed problems for the use of this virus as a gene therapy vector.

11.1 Construction of Plasmids to Test AAV/ITR-Rep Integration System

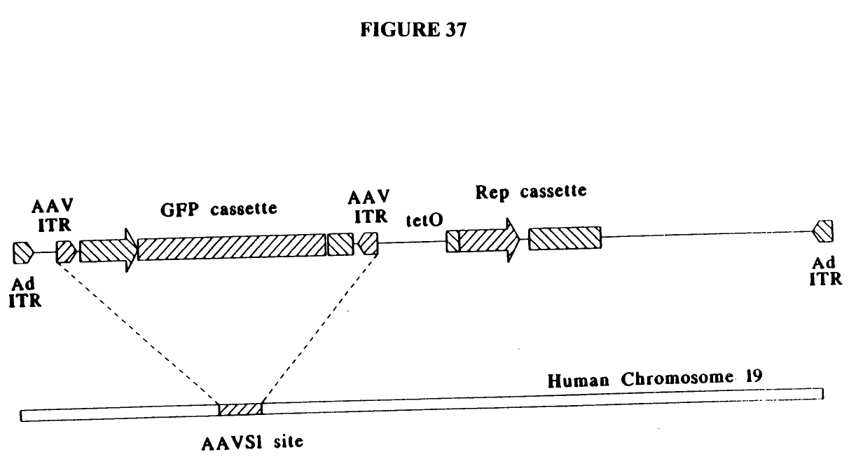

Towards the incorporation of the AAV integration machinery in a mini-Ad vector FIG. 35, Applicants have developed and tested a plasmid vector that contains the Adeno-associated viral elements necessary for integration. In a design of the present invention (FIG. 37), the vector consists of a Rep expression cassette (containing the viral endogenous promoter), as well as a cassette for expression of a reporter gene flanked by two AAV ITRs. The Rep expression cassette was obtained after PCR amplification of sequences 193 to 2216 in the AAV genome from plasmid pSUB201 (41). This fragment starts right after the ITR and extends through the p5 promoter and the Rep78 coding sequence.

11.2 Test of AAV/ITR-Rep Integration System in Culture Cells

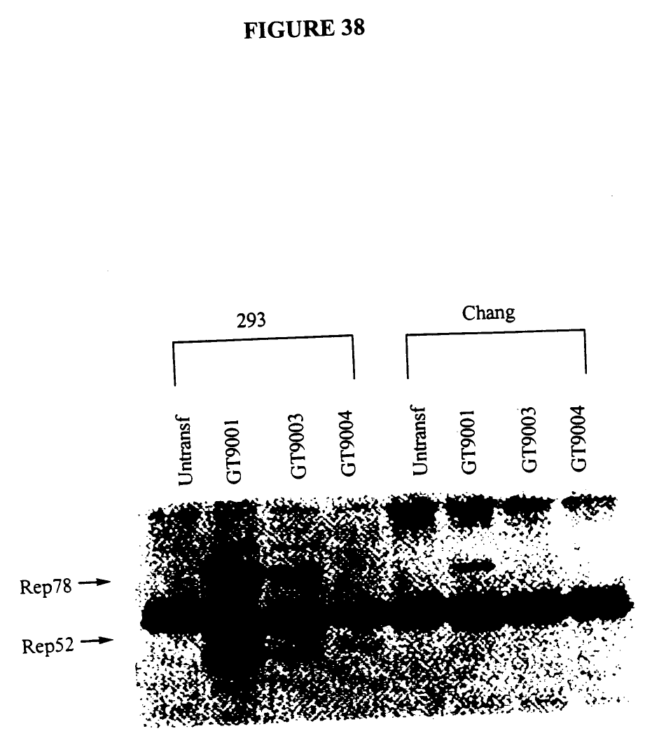

Expression of Rep from this plasmid in transiently-transfected 293 cells and an E1 non-expressing cell line (Chang liver cells) was tested by immunoprecipitation plus Western blot with specific antibodies. The results (FIG. 38) show that two different forms of Rep are produced in 29′ and Chang liver cells. Rep78 appears as a doublet while Rep52, product of p19 promoter, appears as a single band. In Chang liver cells, two major forms are detected, Rep78 also as a doublet and Rep52, although the signal is stronger in 293 cells.

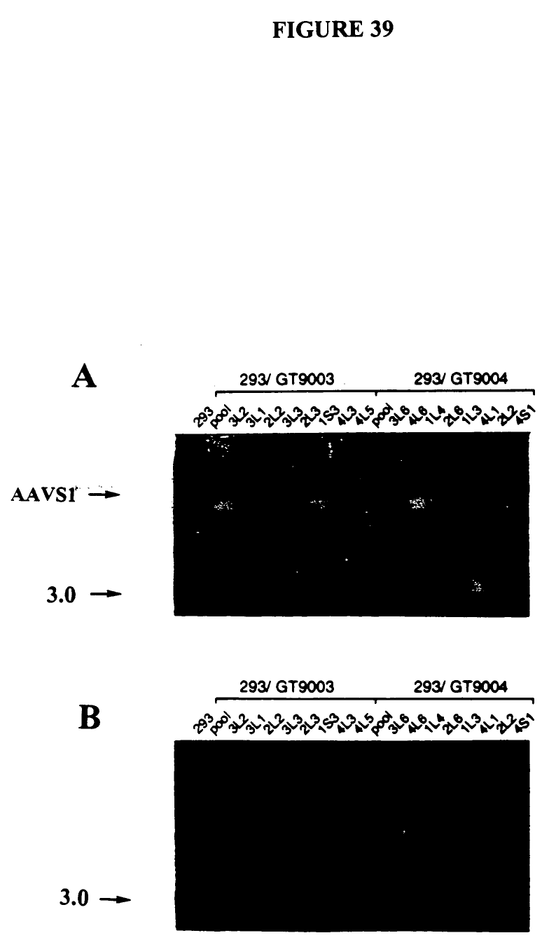

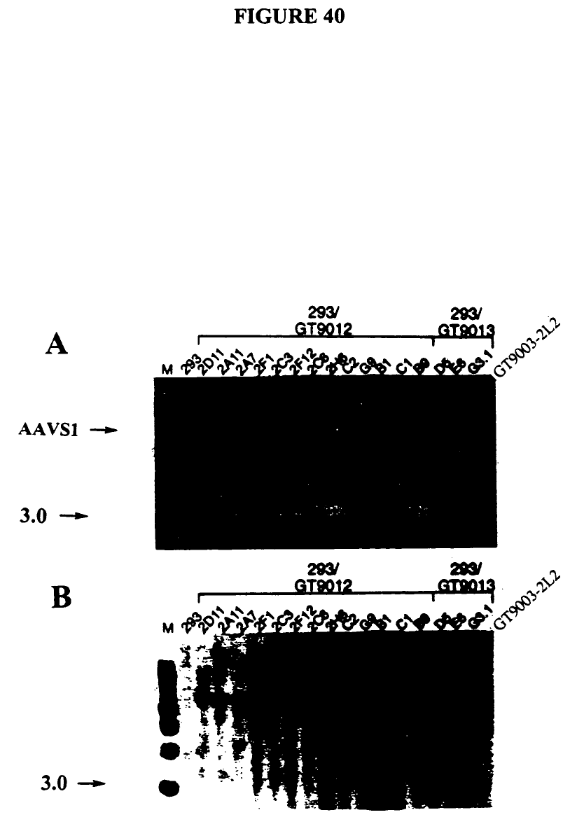

In order to test for the specific integration capability of these plasmids, a control plasmid was constructed by removing the Rep expression cassette, but keeping the reporter gene expression cassette placed between two AAV ITRs. 293 cells were transfected with plasmids GT9003 or GT9004 and then selected for 12 days with G418 (0.5 mg/ml). G418-resistant colonies were isolated, expanded, and genomic DNA was extracted from different colonies by the salt precipitation method (125). Genomic DNA was digested with EcoRI and analyzed by Southern blot with a probe for AAVS1. EcoRI was chosen because the AAVS1 locus is contained within an 8 Kb EcoRI-EcoRI fragment. FIG. 39 (panel A) shows that 50% of the resistant colonies analyzed which derived from plasmid GT9003 (Rep-expressing plasmid) revealed rearrangements of at least one AAVS1 locus, as indicated by the presence of a shifted band in addition to the 8 kb band corresponding to the normal sequence. Rearrangements were not observed in the colonies derived from plasmid GT9004, indicating that this phenomenon is dependent on the expression of Rep. These results suggested that Rep was able to drive specific integration of the transgene. The membrane was then rehybridized to a specific probe for neo (FIG. 40, panel B). The pattern of bands obtained indicated that some AAVS1 rearrangements correspond to neo (ex. clone 2L2) but also suggested that random integration events occurred frequently in the clones analyzed, possibly favored by the selective pressure applied.

11.3 Test of AAV/ITR-Rep Integration System Without Selective Pressure

In order to rule out this possibility, we performed another set of experiments with plasmids GT9012 and GT9013. In these plasmids the reporter gene is GFP (Aequorea Victoria green fluorescent protein). This reporter makes cells suitable for isolation using methods including but not limited to sorting and single-cell cloning by flow cytometry, thereby eliminating effects of selective pressure imparted by the neo expression cassette. 293 cells were transfected with either plasmid. One day after transfection, cells falling into a given range of fluorescence (thus eliminating variability due to differences in transfectability) were sorted by flow cytometry and single-cell cloned in 96-well plates. Two to three weeks after sorting, colonies were scored for fluorescence. Three independent experiments were performed and the results are shown in Table 2-1. The cloning efficiency (number of colonies developed per total number of seeded wells) showed some variability for GT9013-derived cells, but was generally constant for those transfected with plasmid GT9012. Approximately 50% of the colonies derived from plasmid GT9012 were fluorescent and maintained their fluorescence in subsequent passages, whereas 8% of those derived from plasmid GT9013 showed any fluorescence. The fluorescence intensity was dim, an observation consistent with the integration of one or few copies of the GFP expression cassette into the host cell genome. Interestingly, some colonies showed a mosaicism in GFP expression. One explanation for this could be that the integration event occurred after the sorted cell started division giving rise to two different populations. In a parallel experiment cells were transfected with plasmid pCA-GFP that contains a GFP expression cassette alone (no viral sequences). Fluorescent colonies were not detected after sorting plus single-cell cloning (Table 2-1). Taken together, these results indicate that the efficiency of integration is enhanced by the presence of AAV ITRs but is 4-5 fold higher when Rep is expressed. To further analyze targeted integration of GFP in AAVS1, several colonies (fluorescent and non-fluorescent) were grown and genomic DNA was extracted as described above. FIG. 40 (panel A) shows results obtained by Southern blot with a probe for AAVS1. Rearrangements of AAVS1 are indeed detected in several colonies derived from plasmid GT9012 whereas no rearrangement is observed in GT9013-derived colonies, thus indicating that the presence of Rep is necessary for targeted integration. This membrane was then probed for GFP to check the correspondence with the rearranged bands (FIG. 40, panel B). Parental cell line 293 was negative, as expected. Five clones showed bands over 8 Kb matching those obtained with AAVS1, therefore indicating specific integration of GFP in AAVS1. Altogether, the results shown above indicate that plasmids containing AAV ITRs and Rep can integrate at high frequency in the host genomic DNA and suggest that this design is useful for the integration of sequences delivered by adenoviral vectors.

TABLE 2-1
Results of single-cell cloning experiments.

| | | GT9012 | GT9013 | pCAGFP |
|---|---|---|---|---|
| Experiment 1 | Cloning | 92/192 | 116/192 | |
| | efficiency* | (48%) | (60%) | |
| | Integration | 43/92 | 5/116 | |
| | efficiency** | (47%) | (4%) | |
| Experiment 2 | Cloning | 42/96 | 26/96 | |
| | efficiency | (44%) | (27%) | |
| | Integration | 23/42 | 2/26 | |
| | efficiency | (55%) | (8%) | |
| Experiment 3 | Cloning | 93/192 | 25/192 | 51/96 |
| | efficiency | (48%) | (13%) | (53%) |
| | Integration | 37/93 | 1/25 | 0/51 |
| | efficiency | (40%) | (4%) | (0%) |

*indicates number of colonies per number of wells seeded.
**indicates number of colonies showing fluorescent cells two weeks after sorting per number of colonies.

### Example 12

### Site-Specific Integration of the FVIII Expression Cassette

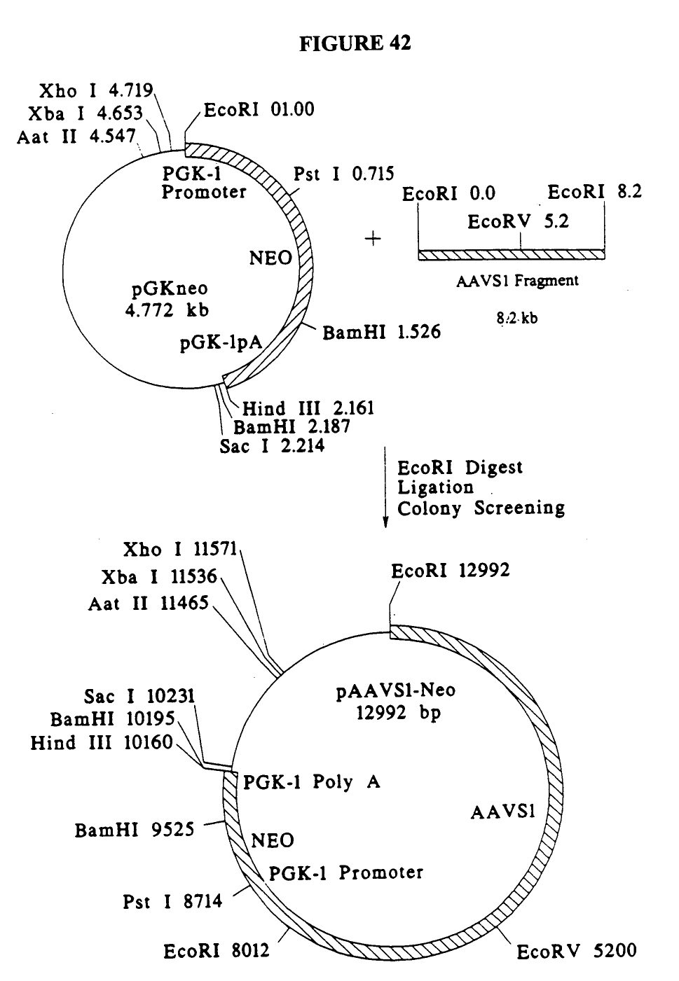

It has been previously shown by the applicants in the parent application (U.S. Ser. No. 08/659,961 filed on May 31, 1996) of the present application that introduction of ITR DNA sequences in a plasmid coupled with Rep78 expression enhances the integration of DNA sequences of interest into the cellular genome (56). The previous invention comprises multiple plasmids comprising an expression cassette having a reporter gene ([i.e., neo or the gene encoding green fluorescent protein (GFP)]) flanked by AAV ITR sequence (hereafter referred to as the integration cassette), in combination with an upstream Rep expression cassette (FIG. 42, panels A-D). Experimental results demonstrate that the integration frequency of these plasmids (i.e., GT9003 and GT9012) is approximately 10 times higher than those plasmids lacking the Rep cassette (i.e., GT9004 and GT9013). The data further indicates that Rep is essential for efficient, targeted integration of exogenous DNA into a host cell genome. In light of these results, the present invention provides a hybrid vector that combines the advantages of the Ad vector (high titer preparation, large capacity for exogenous DNA, high level infectivity of multiple cell types) and the integration capabilities of AAV. This hybrid virus of the present invention replicates as an adenovirus and comprises the AAV elements sufficient for integration.

The present invention comprises a mini-Ad vector having a Rep expression cassette and a FVIII expression cassette flanked by AAV ITRs. Additional exogenous DNA (up to 36 kb) may be inserted into the vector. Additional exogenous DNA of this vector corresponds to human albumin genomic sequences (non-coding). The Rep expression cassette encompasses bp 193 to 2216 bp of the AAV genome. This fragment originates immediately following the ITR and extends through the p5 promoter and the Rep78 coding sequence. For the reasons listed below, a fragment comprising seven tet operators was introduced upstream of the p5 promoter was included to allow for transcriptional repression of the rep gene by the tet-KRAB repressor (42).

It is possible that high levels of Rep protein within a cell would be cytostatic and possibly interfere with replication of the mini-Ad vector. The tet-KRAB repressor, then, may be provided as a transcriptional switch in order inhibit expression of Rep during viral vector generation. To this end, the present invention provides a 293 cell line stably expressing the tet-KRAB repressor protein. Upon entry of virus into the host cell that does not express the tet-KRAB repressor protein, Rep expression occurs due to the absence of the repressor in those cells, thus promoting integration of the sequences flanked by AAV ITRs into the cellular genome. The viral vector thus generated may be tested in vitro and in vivo for the frequency and specificity of integration.

### Example 13

### AAVS1 Cloning and Vector Construction

An embodiment of the present invention is a methodology for the generation of a transgenic mouse harboring the human AAVS1 integration site for use as an in vivo animal model. The animal model is to be used for testing site-specific integration of a viral vector containing the AAV integration mechanism described above. The first step towards development of the animal model was cloning of the AAVS1 site and insertion of the sequence into a mammalian expression vector.

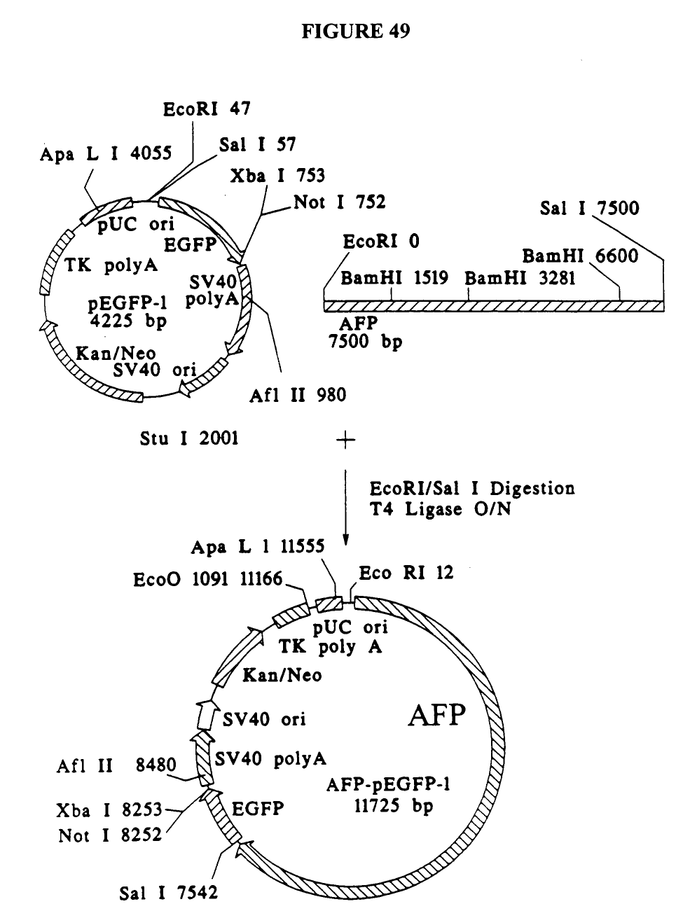

The AAVS1 human integration site was originally cloned by Kotin et.al. (50) as an 8.2 kb EcoRI fragment, of which the first 4067 bp have been sequenced. This DNA sequence information was used to design two oligonucleotide primers, which were subsequently used to generate a 253 bp PCR product for use as an AAVS1-specific probe. The upper primer, U2492 (SEQ ID NO:5: GCTGTCTGGTGCGTTTCACTGAT), is a 23-mer that extends from basepairs 2492-2515 of the AAVS1 sequence and the lower primer, L2722 (SEQ ID NO:6: TCACAAAGGGAGTTTTCCACACG), also a 23-mer extends from basepairs 2722-2745 of the AAVS1 sequence. PCR amplification was performed using 100 ng human genomic DNA as template and a 1 μM final concentration of the U2492 and L2722 primers (SEQ ID NO:5 and SEQ ID NO;6, respectively) as follows: 95° C., 4 min-1 cycle; 95° C., 0.5 min, 55° C., 0.5 min, 72° C., 1 min-35 cycles; 72° C., 7 min-1 cycle. The 253 bp AAVS1-specific PCR product was sent to Genome Systems (St.Louis, Mo.) where it was used to screen a human P1 genomic DNA library. Four P1 clones (#6576, 6577, 6578, 6579), which ranged in size from 80-120 kb, were identified which yielded the correct 253 bp AAVS1 PCR fragment. To confirm that these clones contained the AAVS1 sequence, DNA was isolated from the 4 P1 clones, digested with either EcoRI only or EcoRI in combination with EcoRV, and used for Southern blot analysis. Hybridization of the blot using the 253 bp AAVS1-specific PCR product as a probe, revealed that all clones contained the expected 8.2 kb EcoRI fragment (FIG. 49A) and the expected 5.2 kb EcoRI/EcoRV fragment (FIG. 49B), indicating that they contained an intact, full-length copy of the human AAVS1 sequence. The 8.2 kb EcoRI fragment was isolated from P1 clone #6576 and cloned into the EcoRI site of the mammalian expression vector. pGKneo. The resulting 12.9 kb plasmid, pAAVS1-Neo, harbors the neomycin resistance gene (Neo) for selection in mammalian cells, as well as the human AAVS1 sequence (FIG. 42).

### Example 14

### Generation and Characterization of AAVS1 (+) ES Cell Lines

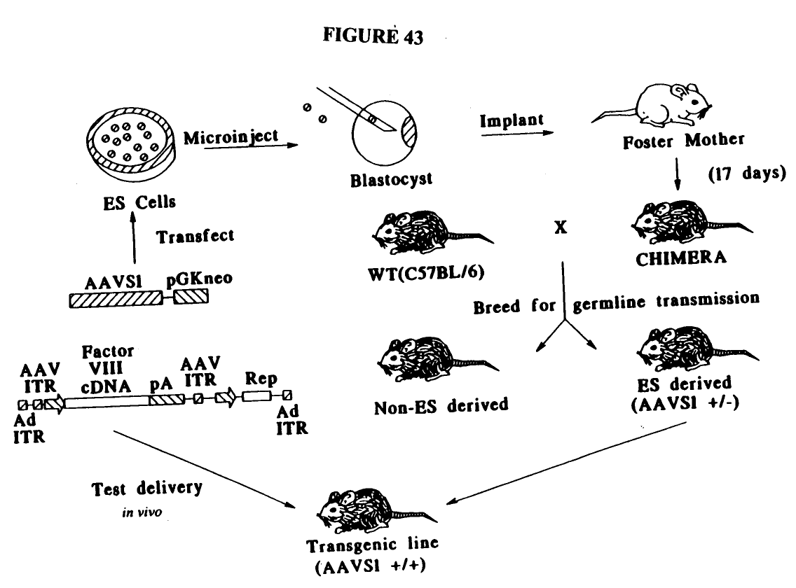

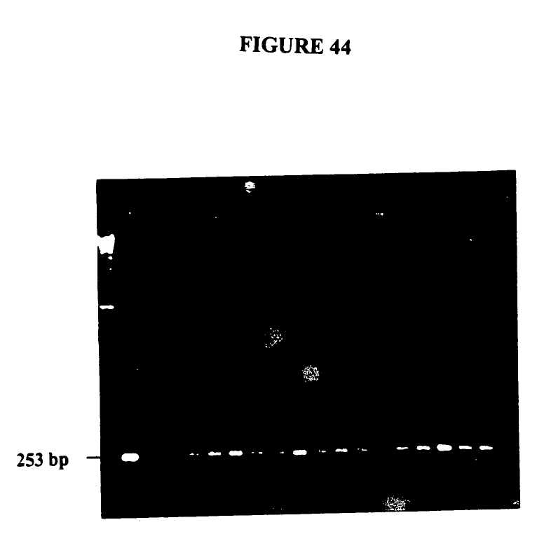

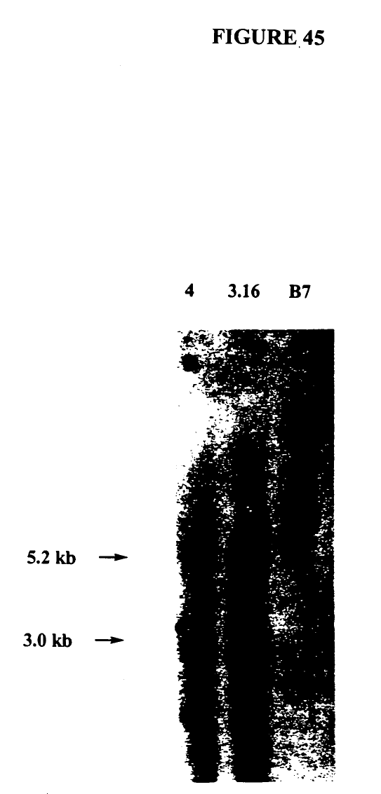

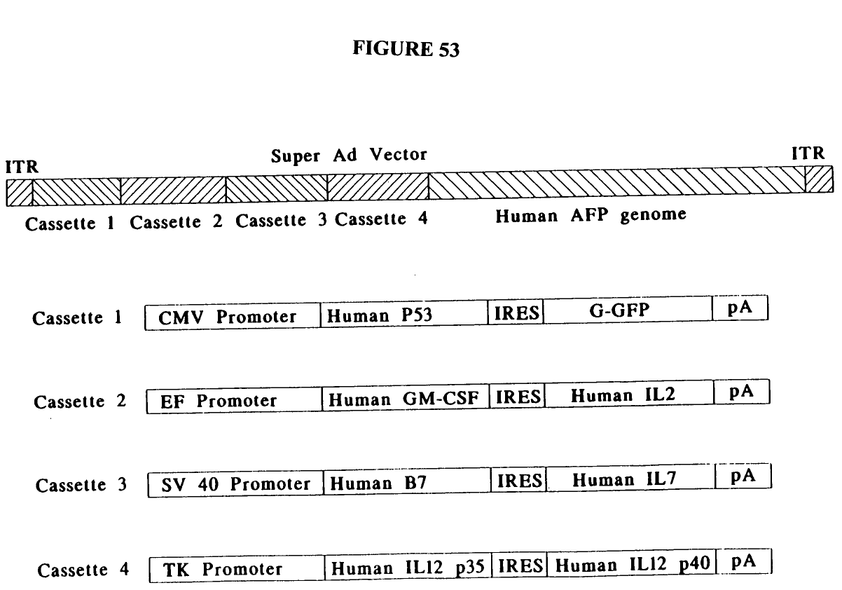

To generate a mouse embryonic stem (ES) cell line comprising the AAVS1 sequence for use in the generation of an AAVS1 transgenic mouse, the pAAVS1-Neo plasmid was transfected into mouse ES cells (129 Sv agouti, Genome Systems) (FIG. 43). 25 ug of pAAVS1-Neo plasmid DNA was linearized with XbaI and transfected by electroporation (975 uFd, 252 v.) into ES cells using a Biorad Gene Pulser. Transfected cells were selected for one week in 250 ug/ml G418. 24 neo-resistant (Neo^{R}) colonies were isolated, expanded, and characterized by morphology to obtain clones which were >95% “un-differentiated” in order to enrich for cell lines that maintained a totipotent phenotype. Genomic DNA was isolated from 17 Neo^{R} ES clones, as well as from the untransfected, parental ES cell line, and 100 ng of the DNA utilized as template using primers U2492 and L2722 (SEQ ID NO:5 and SEQ ID NO;6, respectively; final concentration of 1 μM) for AAVS1-specific PCR. The conditions for PCR were: 95° C., 4 min-1 cycle; 95° C., 0.5 min, 55° C., 0.5 min, 72° C., 1 min-35 cycles; 72° C., 7 min-1 cycle. As shown in FIG. 44, PCR of DNA from 17/17 Neo^{R} ES clones generated the expected 253 bp AAVS1 PCR product, while PCR analysis of DNA from the untransfected control ES cells did not generate detectable PCR product. Southern blot analysis was performed on control and AAVS1 (+) ES cell lines to confirm that a functional AAVS1 sequence had been preserved following transfection and genomic integration (FIG. 45). Genomic DNA from each of the AAVS1 positive (as assessed by PCR) ES cell lines (AAVS1 ES#4 and ES#3.16) was digested with EcoRI in combination with EcoRV, electrophoresed, blotted, and hybridized with an 8.2 kb AAVS1 probe. Both ES#4 and ES#3.16 cell lines contained the expected 5.2 kb and 3.0 kb EcoRI/EcoRV fragments, indicating integration of the entire 8.2 kb AAVS1 sequence (FIG. 53). The untransfected parental ES cells showed no hybridizing bands using this human AAVS1-specific probe. It was expected that some hybridization would be detected in the control mouse ES cells, since it is known that the AAV virus integrates into the mouse genome as well as in human, however, the mouse homologue for AAVS1 (which has not yet been identified) must be significantly divergent from the human sequence in that it does not cross-hybridize with a human AAVS1 probe. Each of these AAVS1 (+) cell lines, ES#4 and ES#3.16, were used for subsequent blastocyst microinjection experiments towards the production of AAVS1 transgenic mice.

### Example 15

### Production of AAVS1 Transgenic Mice

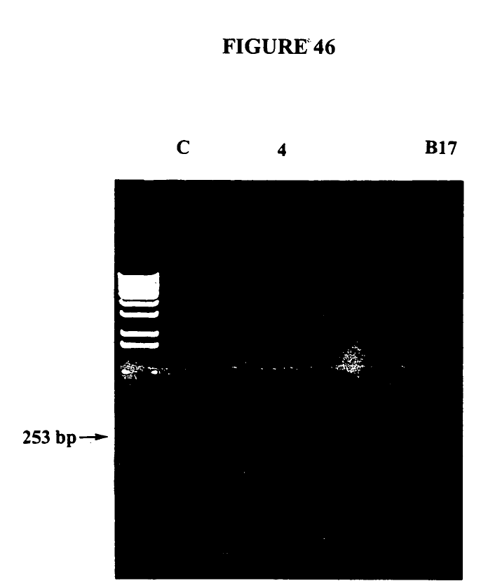

The AAVS1-positive ES clones, ES#4 and ES#3.16, were grown on 1° murine embryonic fibroblast feeder layers, in the presence of Leukemic Inhibitory Factor (LIF—an anti-differentiation factor), and maintained at very low passage (P.2-P.7) in order to preserve an undifferentiated, totipotent phenotype. Blastocyst-stage embryos were collected at Day 3.5 p.c. from superovulated, C57BL/6 mice, maintained in M16 embryo medium. 15-20 ES cells (AAVS1 ES#4 or ES#3.16) and microinjected into the blastocoel cavity of the 3.5 day embryos using a Leitz DM-ILB Microinjection Workstation. Following microinjection, the embryos were returned to M16 medium and incubated in 5% CO₂, 37° C. for 2 hours to allow the blastocysts to re-cavitate. 10-15 injected blastocysts were subsequently transferred into the uterus of Day 2.5 post-coitus (p.c.), pseudopregnant, CB6F₁ foster mothers. Following uterus transfer, the blastocysts implant into the uterine wall, the AAVS1-positive ES cells become incorporated into the embryo's inner cell mass, and contribute their genetic information to the developing embryo, resulting in the birth of transgenic (chimeric) progeny approximately 17 days later. To date more than 40 high-percentage, male chimeras (founders) have been produced. Examples of some of these chimeric founders, demonstrating high-percentage contribution of the ES cell-derived agouti (brown) coat-color genes were obtained. PCR analysis was performed for detection of the AAVS1 transgene in chimeric mice. Genomic DNA was isolated from the tails of AAVS1 chimeras and from non-chimeric littermates, and 100 ng of the DNA screened by PCR analysis using the AAVS1-specific primers, U2492 and L2722 (SEQ ID NO:5 and SEQ ID NO;6, respectively) at a final concentration of 1 μM. The conditions for PCR were: 95° C., 4 min-1 cycle; 95° C., 0.5 min, 55° C., 0.5 min, 72° C., 1 min-35 cycles; 72° C. 7 min-1 cycle. The correct 253 bp AAVS1 PCR product was indeed detected in tail DNA from a high-percentage chimera (FIG. 46, lane 7), but was not detected in the tail DNA of a non-chimeric littermate or in a low percentage chimera with less than 10% agouti coat color chimerism. Thus human AAVS1 integration sequence has been successfully cloned, and transfected into mouse embryonic stem (“ES”) cells. The transfected ES cells were then microinjected into blastocyst-stage embryos, and demonstrated the presence of this human transgene in the genome of the resultant transgenic mice. These chimeric founders are then bred with wild-type C57BL/6 females to obtain germline transmission of the human AAVS1 transgene. Once germline transmission is achieved, the F₁ heterozygote progeny are bred resulting in a homozygous AAVS1 transgenic mouse line. This homozygous line may then be used to test AAVS1 site-specific integration of either AAV viral vectors, hybrid adenovirus/AAV viral vectors, or any other plasmid vector comprising the AAV ITRs and Rep 78 68 genes necessary for integration at the AAVS1 site. The AAV transgene vectors may be delivered in vivo to the AAVS1 transgenic mice either by viral infection (following I.V. injection) or by using ligand-mediated DNA/liposome complexes. The frequency of site-specific integration, stability of the integrated transgene and the duration of stable protein expression (i.e. human Factor VIII or Factor IX) may then be assessed following integration into the target cells.

The efficiency of in vivo delivery of a viral vector including the adeno-associated integration element to a transgenic mouse having the AAVS1 sequence incorporated into its genome comprising may be tested by the following method. A viral vector of the present invention is injected into the intravenous or portal vein of the transgenic mouse. The vector may be part of a pharmaceutical composition and may or may not be complexed with lipid such as Lipofectamine (GIBCO/BRL) and/or a liver-specific ligand (79, 80). Following administration of the viral vector to the mouse, blood samples are taken weekly for up to a year or more from the tail vein to assess the duration of transgene expression. The level of expression of the effector or reporter gene of the viral vector is measured using a technique such as northern blot, RNase protection analysis, or PCR. In testing the FVIII mini-Ad, FVIII is detected by ELISA assay. The level of expression of the effector or reporter gene in each blood sample is compared to one another in order to determine the duration of transgene expression. Also, in order to determine site-specific integration of the vector, genomic DNA is isolated from the liver tissue of the animal. PCR analysis of the genomic DNA using an AAVS1-specific primer and a primer containing sequence homologuos to sequence of the vector is then performed. Site-specific integration of the vector at the AAVS1 site of the genome of the transgenic animal produces a product containing both AAVS1 and vector sequences. The amplified PCR product, provided the viral vector integrated into the AAVS1 site of the animal, includes vector sequence.

### Example 16

A. FVIII Transgenic Mouse Harboring the Human AAVS1 Integration Sequence

An AAV/ITR-Rep vector comprising either the neo or GFP reporter gene (GT9003 and GT9012, respectively) was transfected into human 293 cells. Extracts of these cells were then assayed by Southern blot for site-specific integration of the vector at the endogenous AAVS1 site. Integration at AAVS1 was observed at a frequency of 50% in samples obtained following transfection for either the neo or GFP version of the AAV-ITR/Rep vector (GT9003 or GT9012, respectively). These results indicate that the AAV ITRs and Rep coding sequences are sufficient to direct high-efficiency, site-specific integration at AAVS1 (56). To demonstrate that the mini-Ad-hFVIII vector containing the AAV-ITR/Rep integration element is targeted to AAVS1 in vivo, integrates in a site-specific manner and maintains long-term expression of hFVIII, an animal model system was developed.

The present invention comprises a transgenic mouse harboring the human AAVS1 integration site within its genome. The transgenic mouse is generated using embryonic stem cell manipulation technology (43) as illustrated in FIG. 43. An expression vector comprising the entire 8.2 kb human AAVS1 sequence and neo (or Neo) selection marker is constructed. The AAVS1/Neo vector is transfected into totipotent mouse embryonic stem (ES) cells to obtain Neo^{R}, AAVS1⁺ ES cell clones that are subsequently microinjected into mouse blastocyst-stage embryos and implanted into the uterus of a foster mother. Following implantation, the AAVS1⁺ ES cells resume normal embryonic development and contribute their genetic information (including the human AAVS1 sequence) to the developing embryo. Chimeric (transgenic) progeny are identified by the presence of ES cell-derived agouti-brown coat color. Chimeric founders are then bred with wild-type C57BL/6 mice to obtain germline transmission of the transgene. F1 heterozygotes are bred to obtain a homozygous mouse line which has stably incorporated the human AAVS1 integration sequence into its genome. This mouse model is then injected, via the tail vein or portal vein, with the AAV-ITR/miniAd-FVIII vector to assess in vivo transduction efficiency, integration at the human AAVS1 sequence, and duration of transgene expression.

B. A Transgenic Mouse Tolerized to Human FVIII

Also provided by the present invention is a FVIII-tolerized mouse model system. In the past, FVIII tolerization has been achieved by transient injection of FVIII into neo-natal mice (44). The present invention comprises a mouse having the human FVIII gene under the control of a promoter that functions in a developmental stage-specific manner. Such a promoter may include but is not limited to that of the α-fetoprotein gene or the embryonic globin gene, epsilon. The α-fetoprotein promoter is an example of an early developmental stage-specific promoter that is inactive in the mature animal (45). The embryonic globin gene, epsilon, is another example of a developmentally regulated gene that may be utilized in the present invention. Under the control of the α-fetoprotein promoter, gene expression is limited to the liver and is dependent upon liver specific transcription factors for activation (46, 47). As the normal site of FVIII expression and the preferred target organ for FVIII gene delivery is the liver, a liver specific promoter element that is also developmentally regulated would be preferred. The α-fetoprotein promoter (AFP) meets both of these criteria. The α-fetoprotein promoter does not function in undifferentiated ES cells but is induced during differentiation (48); as such, it may be utilized to control hFVIII expression in transgenic nice.

An objective of the present invention is to provide a transgenic mouse that has been tolerized to the xenogenic human FVIII protein. The transgenic mouse may be utilized to test delivery of human FVIII in vivo using adenoviral or AAV vector systems, or using FVIII-secreting cells in an immunoisolation device. In this system, human FVIII is expressed embryonically in the developing transgenic mouse under control of the AFP promoter. HFVIII, then, is seen as “self” by the mouse and tolerance occurs. As the mouse is tolerized to hFVIII, an immune reaction does not occur toward the xenogenic human FVIII transgene product when it is delivered by the therapeutic vector (i.e., the AAV-mini-Ad-FVIII). In this manner, an accurate assessment of the antigenicity of the viral vector backbone, as well as a reliable measurement of the duration of gene expression in vivo. Also, as the AFP-FVIII transgene is expressed only during embryogenesis (and not after the animal matures), accurate levels of hFVIII delivered to the liver of the mature transgenic mouse.

### Example 17

### Construction of AFP-FVIII-Neo Vector

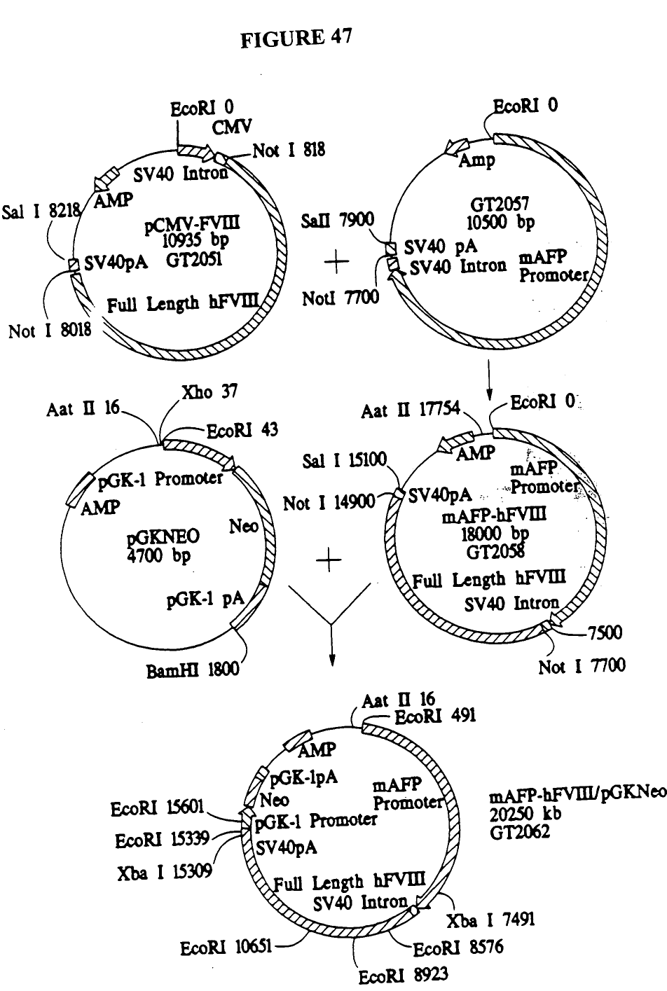

To generate a vector capable of driving expression of human Factor VIII in a temporally-regulated, embryo-specific fashion in a cell in order to achieve in utero tolerization of a transgenic mice, the mouse α-fetoprotein (AFP) promoter was utilized. The plasmid GT2057 comprises a 7.5 kb AFP promoter sequence originally characterized by Urano et.al. (33). A 7.2 kb Not I fragment, containing the full-length human Factor VIII gene, was isolated from pCMV-FVIII (GT2051) and cloned into GT2057 behind the AFP promoter at the Not I site (FIG. 47). The cassette containing the AFP promoter and the hFVIII gene was subsequently cloned as an Aat II/Sal I fragment into the Neo expression vector, pGKNeo, at Aat II/Xho I. The resultant 20.2 kb vector, mAFP-hFVIII-pGKNeo (FIG. 47), harbors the hFVIII gene under control of an embryonic promoter (AFP), and has a Neo expression cassette for selection in mammalian cells.

### Example 18

### Generation and Characterization of AFP-FVIII (+) ES Cell Clones

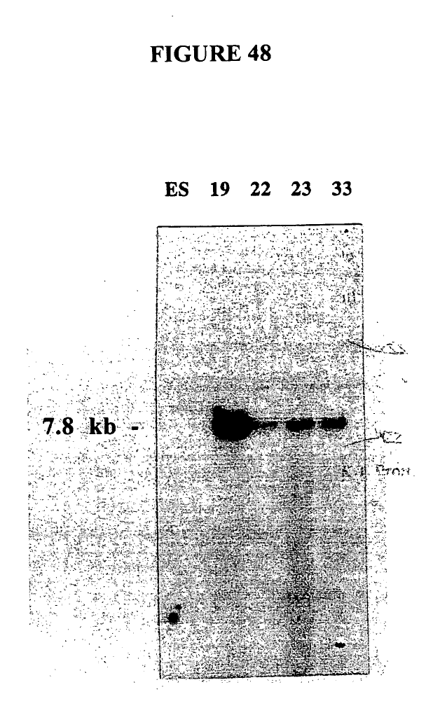

In order to generate a mouse embryonic stem (ES) cell which contains the AFP-hFVIII sequence, for use in the production of transgenic mice, the mAFP-hFVIII-pGKNeo vector was stably transfected into mouse ES cells. 20 ug of AFP-FVIII-Neo DNA was linearized with Aat II and electroporated into ES cells (975 uFd, 252v.) using a BioRad Gene Pulser. Following electroporation, ES cells were propagated on embryonic fibroblast feeder layers in 250 ug/ml G418 to select for Neo^{R} clones. 48 Neo^{R} clones were picked, expanded, and analyzed for functional Factor VIII protein using a Coatest kit. No FVIII was detected in tissue culture supernatants from any of the 48 clones. Genomic DNA was isolated from 13 Neo^{R} ES clones and from untransfected parental ES cells, digested with Xba I, and screened by Southern blot analysis using the 7.2 kb FVIII Not I fragment from GT2051 as a probe. 11/13 transfected clones contained the expected 7.8 kb Xba I fragment, confirming the presence of the entire hFVIII sequence as well as 500 bp of the 3′ end of the AFP promoter. Analysis of four of these AFP-FVIII (+) ES clones, as well as the parental ES cell control, is shown in FIG. 48. These results indicate that the hFVIII transgene was present in the Neo^{R} clones.

To test whether the AFP promoter was functional in ES cells, the AFP promoter was cloned as an EcoR1/Sal I fragment into the reporter plasmid. pEGFP-1 (Clontech), to drive expression of the Green Fluorescence Protein (GFP; see FIG. 49). The pAFP-EGFP-1 plasmid was transiently transfected into both ES cells and HepG2 cells (a liver cell line known to express high levels of α-fetoprotein), and examined by direct visualization using a Nikon Diaphot broad range microscope with a FITC filter after 24 hours for green fluorescent cells. GFP expression was detected in the HepG2 cell line but not in mouse ES cells (data not shown), confirming that the AFP promoter does not function in undifferentiated ES cells, but on in a differentiated liver cell line. These results are in agreement with those of Vogt et.al. (48), which showed that the AFP promoter was silent in undifferentiated F9 embryonic stem cells, and activated when induced to differentiate following treatment with retinoic acid. To confirm that the mAFP-hFVIII-pGKNeo vector was functional in a cell line where expression would be expected, the mAFP-hFVIII-pGKNeo vector was transfected into HepG2 cells and 10×-concentrated supernatants were analyzed for hFVIII protein expression using the chromogenic Coatest FVIII assay (Pharmacia, Piscataway, N.J.). Human FVIII was detected at the level of 3.0 ng/10⁶ cells/24 hrs., confirming that the AFP-FVIII construct was functional and that the tissue-specific and developmental-specific expression pattern of the 7.5 kb AFP promoter/enhancer element was preserved. Having demonstrated that the AFP-FVIII vector was functioning properly, one of the AFP-FVIII (+) ES clones, clone #22 (FIG. 48, lane 3), was chosen, based on its undifferentiated growth phenotype, to use for blastocyst microinjection experiments.

### Example 19

### Production of hFVIII-Tolerized Transgenic Mice

The present invention provides a transgenic mouse that has been tolerized in utero to the xenogeneic human Factor VIII protein. This transgenic mouse is used to test delivery of human FVIII in vivo using adenoviral or AAV vector systems, or using FVIII-secreting cells in the TheraCyte immunoisolation device such as that described in U.S. Pat. No. 5,314,417; 5,344,454; 5,421923; 5,453,278; 5,545,223; or 5,569,462. As hFVIII is expressed embryonically in the developing transgenic mouse under the control of the AFP promoter, hFVIII as delivered by the therapeutic vector (i.e. the AAV-miniAd-hFVIII) is recognized by the immune system of the mouse to a “self” antigen. As such, tolerance to the hFVIII protein results. As the transgenic mouse is tolerized to hFVIII, an immune reaction to the “xenogeneic” human FVIII protein will not occur, and an accurate assessment of antigenicity of the viral vector backbone and a realistic measurement of the duration of gene expression in vivo may be determined. Also, as the AFP-FVIII transgene is expressed mainly during embryogenesis, the amount of hFVIII protein expressed by the liver as a result of transduction by the vector in mature transgenic mice may be accurately quantitated.

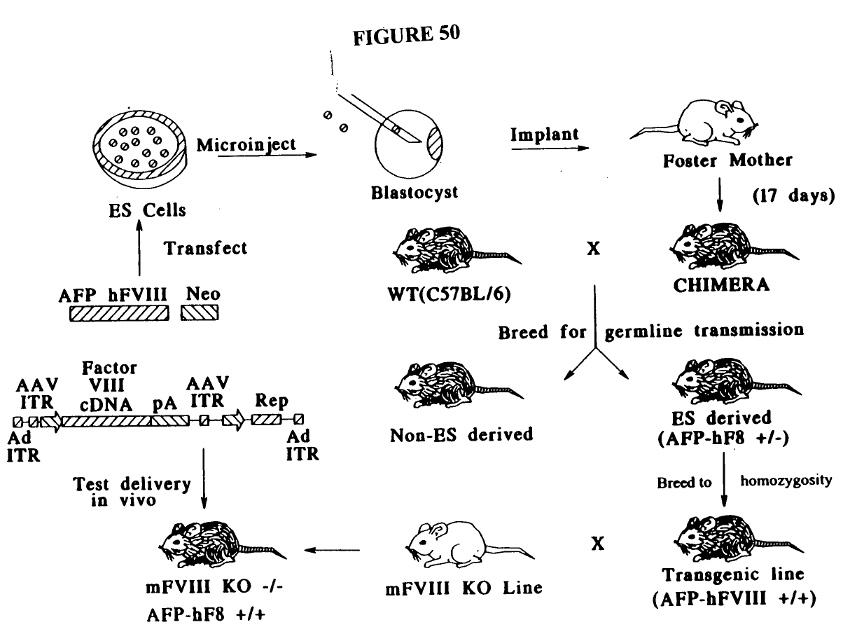

The scheme for the generation of hFVIII-tolerized transgenic mice is outlined in FIG. 50. ES cells from the AFP-FVIII (+) ES clone #22, were microinjected into C57BL/6 blastocysts, and implanted into the uterus of foster mothers. To date, four chimeric progeny have been produced using ES clone #22. They are mated with wild-type C57BL/6 mice to test for germline transmission, and germline founders bred to obtain a homozygous AFP-hFVIII transgenic mouse line. Since the chimeric progeny, by definition, have mosaic expression of the AFP-hFVIII transgene in all of their tissues, they are also used directly for in vivo gene delivery experiments, without having to wait for production of a homozygous line. Transgenic animals produced by this scheme, are initially challenged by an injection of hFVIII protein, bled, and screened for antibodies to the human protein, to ensure tolerization to hFVIII. The AFP-hFVIII-tolerized transgenic mice will also be tested for “leaky” expression of hFVIII in the adult animal. If a small amount of hFVIII protein is produced in adult transgenic animals, it is accurately quantitated so that it can be subtracted from the levels of hFVIII delivered by the therapeutic vector or protein delivery device. Provided the transgenic animal is tolerized to hFVIII and expresses insignificant levels of endogenous human protein, it can be used to test the efficiency of in vivo delivery of hFVIII, the duration of gene expression, tissue distribution, and immune reactions to elements of the delivery system, other than the transgene (i.e., vector backbone, viral coat proteins) may be analyzed. Other parameters may also be tested using the transgenic animal.

### Example 20

### Second Generation Transgenic Animal Models

a. Breeding of AFP-hFVIII Tolerized Mouse with A Mouse FVIII Knockout

A transgenic mouse strain with a targeted disruption (gene knockout) of the mouse Factor VIII gene has been obtained through a non-exclusive, restricted-use license agreement with John Hopkins University and The University of Pennsylvania. This mouse line has severe mFVIII deficiency and thus is a useful model for hemophilia A (51). By crossing our transgenic mouse tolerized to human Factor VIII with a mouse that is totally deficient for mouse Factor VIII, it is possible to produce a “clean” model for testing in vivo delivery of hFVIII. In the absence of mouse FVIII protein having the potential to cross-react with hFVIII, an accurate quantitation of hFVIII. In addition, this doubly-transgenic mouse provides a useful model for the phenotypic correction of hemophilia A using gene therapy.

b. A Triple Transgenic Mouse

A further embodiment of this invention involves the crossing of all three above-mentioned transgenic animals to produce a “triple-transgenic” mouse model. The mouse described in the previous section, which is tolerized to human FVIII and deficient in mouse FVIII, is cross bred with the AAVS1 transgenic mouse line. This triple transgenic mouse model is preferredly suited for testing all aspects of our AAV-miniAd-hFVIII vector system including: site-specific integration at AAVS1 via the AAV ITR/Rep integration system; delivery and long-term expression of the human FVIII transgene without immune reaction to the tolerized transgene; accurate quantitation of delivered hFVIII due to a lack of adult expression of human FVIII as well as a lack of cross-reacting mouse FVIII protein; and finally, genetic and phenotypic correction of severe FVIII deficiency (hemophilia A).

c. A Transgenic Mouse Tolerized to Green Fluorescence Protein (GFP)

It is convenient to incorporate the GFP expression cassette into the various virus vectors as a reporter gene in new mini-Ad vectors, AAV vectors, and novel versions of helper virus are developed. Viral infection, expansion, helper complementation and in vivo delivery to target cells is easily followed by visual detection of green fluorescence. It has been shown that immune responses to transgene-encoded proteins can negatively impact the stability of gene expression following injection of adenovirus vectors (30). In order to eliminate immune responses to the GFP transgene incorporated into the vector, which could shorten the duration of GFP expression after injection into mice, a GFP-tolerized transgenic mouse is developed. The AFP-EGFP-1 vector described in FIG. 58, or a similar vector comprising the Rat Insulin Promoter (RIP) for pancreas-specific expression of GFP, could be used for this purpose.

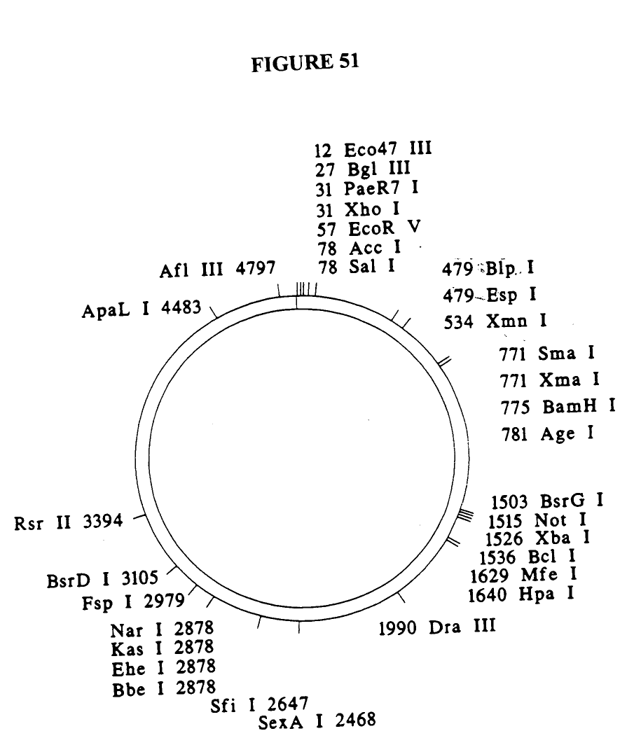

A RIP-EGFP-1 vector (FIG. 51) was used to transfect mouse ES cells in order to develop a stable, Neo^{R} RIP-GFP ES cell line [RIP-GFP(+) ES]. RIP-GFP(+)ES cells are utilized to generate a GFP-tolerized transgenic mouse, in a manner identical to that described for the generation of an AFP-hFVIII tolerized mouse model, substituting the RIP-EGFP-1 vector for the AFP-hFVIII vector. The RIP-GFP tolerized mouse thus produced provides a useful research tool for the development of novel adenovirus vectors or other delivery systems that utilize the GFP transgene as a reporter.

All of the above-described transgenic animal models, including the AAVS1 transgenic mouse provided in Example 12, the hFVIII-tolerized transgenic mouse of Example 15, and the GFP-tolerized transgenic mouse of Example 19C, may be alternatively generated by direct DNA injection of the transgene (pAAVS1-Neo, mAFP-hFVIII-pGKNeo and RIP-EGFP-1, respectively). This is accomplished by injection of the transgene into the male pronucleus of mouse single cell ova to produce transgenic mice, as an alternative to using the ES cell technology descirbed above. To one skilled in the art, this is an obvious alternative method for producing a transgenic mouse. The present inventions, therefore, may be produced by either of the methods discussed in this application (57-61).

### Example 21

### Episomal Mini-Ad Vectors

As another approach to provide elements that will allow long-term expression of the transgene delivered by the mini-viral vector, the present invention provides designs for a site-specific recombinase-based system that permits excision of an auto-replicative episome from the mini-viral sequences upon infection of target cells.

Site-specific recombinases have been extensively used to manipulate DNA. Site-specific recombinases catalyze precise recombination between two appropriate target sequences, cleaving DNA at a specific site and ligating it to the cleaved DNA of a second site (for a review, see Ref. 111). Several systems have been identified and characterized such as the cre/loxP system from bacteriophage P1 (111) or FLP/FRT from yeast (112) (FIG. 3B). The recognition sites (loxP and FRT) for both recombinases (cre and FLP) share a common structure: they have two inverted repeat elements (recombinase binding site) flanking a central core region (site of crossing-over). The orientation of the target sites (as defined by the core region) is responsible for the final outcome: recombination between two parallel sites on the same molecule results in excision of intervening sequences generating two molecules, each one with a target site. Recombination between two antiparallel sites results in inversion of the intervening sequence. Recombination between two parallel sites in diferent molecules results in the integration of sequences flanked by target sites. Since excision is an intramolecular event, it is favored over integration.

In the design of the present invention, recombinases will be used to excise sequences having a eukaryotic origin of replication (ori). Mammalian ori sequences and binding factors have not been characterized to date. However, some viral ori sequences and viral proteins required for initiation of replication have been characterized and incorporated in plasmid vectors, some examples of which including but not limited to SV40 ori/T-Ag from simian virus 40 (113) and oriP/EBNA-1 from Epstein-Barr virus (114). These elements have allowed the generation of plasmids that replicate autonomously in eukaryotic cells and are stably maintained upon selective pressure. Plasmids containing oriP and expressing EBNA-1 protein replicate once per cell cycle (115, 116) and are lost when selective pressure is removed from cells in culture. However, there is no in vivo data about the stability of episomal plasmids in nondividing cells, such as hepatocytes. One should expect that in nondividing cells (i.e. differentiated cells) and without selection, an episome could remain stable for a long period of time. It is believed by the inventors of the present invention that the incorporation of ori sequences in the mini-viral DNA will permit extended expression of the transgene in nondividing cells.

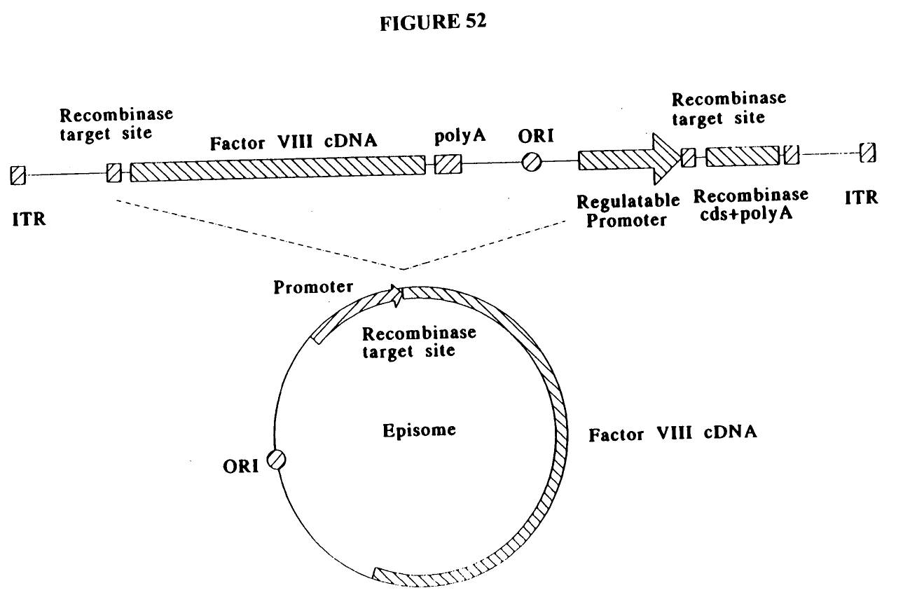

The episomal minivirus elements include but are not limited to (FIG. 52):

a) Recombinase expression cassette: recombinase must be expressed only in target cells, because inappropriate expression in the cells used to generate the virus will promote the excision of the sequences contained between two recombination sites. For this reason, expression is tightly controlled by either adding binding sequences for transcriptional repressors upstream of the promoter (for instance, tetO) or through the use of tissue-specific promoters (ex: albumin promoter, factor VIII promoter).

b) Origin of replication (ori): must include the sequence to initiate or begin replication of DNA and any other element required for replication (ex: DNA binding protein recognizing origin sequences).

c) Transgene: may be any therapeutic or reporter gene flanked by a recombination site (5′) and a polyA signal sequence (3′). It will be expressed only in target cells upon circularization of the DNA.

d) Recombinase target sites: two sites are necessary in parallel orientation, one being placed between the promoter and the recombinase cDNA and the other upstream of the therapeutic gene cDNA.

e) Adenovirus ITRs: necessary for replication and packaging of the minivirus.

f) Stuffer DNA sequence: if necessary to increase the size of the minivirus up to a packageable length. The stuffer DNA sequence may be any DNA fragment of any length.

Under this design, the recombinase is not expressed while amplifying the minivirus. When the mini-viral vector is delivered to target cells, the promoter is functional, recombinase is expressed and the sequences contained between two recombinase target sites are excised and circularized. The recombinase promoter turns into the transgene promoter and the presence of the origin of replication allows stable maintenance of the plasmid, therefore assuring stable expression of the transgene.

### Example 22

### Design of the Mini-Ad for Treatment of Cancer

Currently, one of the most effective approaches to the treatment of cancer using gene therapy is to alter the tumor-host relationship and facilitate the recognition and destruction of malignant cells using the immune system. In the tumor bearing individual, the lack of an effective immune response may be due in part to either weak tumor cell immunogenicity, lack of immune co-stimulation, or a tumor-specific immunosuppressive environment. Cytokine-mediated gene transfer of tumor cells offers one strategy to augment the immune system to mount a more effective antitumor response (117). In recent years a number of cytokine genes have been isolated, cloned and characterized. Systemic administration of certain of these immunomodulators, such as IL-2, has resulted in a proportion of antitumor responses. However, toxicities have accompanied the use of many of these biologics owing to the high concentrations needed to generate clinical effects. The combination of significant undesired effects and marginal therapeutic outcomes from systemic administration has stimulated efforts to genetically engineer tumor cells to produce the cytokines themselves (118).

In animal models, gene modified tumor cells have been used as vaccines to stimulate antitumor responses (117, 119). The appeal of tumor directed cytokine gene transfer is that the cytokine, produced locally, is immunologically more efficient and does not cause systemic toxicity. Tumor antigens expressed on neoplastic cells presented with high local concentration of the cytokine(s), would create an immunological microenvironment impossible to reproduce with exogenous cytokine administration. This immunological microenvironment created by the cytokine producing tumor cells has been efficient in generating cytotoxic T lymphocytes. In a number of different animal models, cytokine producing tumor cells have been shown to be effective in decreasing the tumorgenicity and increasing the expression of immunologically important molecules (117, 119). The initial antitumor rejection appears to be accompanied by a nonspecific inflammatory response. However, rejection of cytokine secreting tumor cells has in most instances led to the generation of systemic, tumor specific immunity that is T cell dependent.

A requirement for preexisting tumor immunogenicity has not been established for most gene transfer models; however many well-characterized tumor cell lines are highly differentiated and immunogenic. In some systems, nonimmunogenic tumors have been shown to generate immunity after cytokine gene transfer. Furthermore, most tumor directed gene transfer models do not lend themselves to investigations in which the host is treated in the presence of an existing tumor burden because the rapid growth of these malignancies provides little time for immunotherapeutic intervention (119).

Recent research has demonstrated that the reduction of TGFβ secretion by tumor cells may be a significant approach to cancer gene therapy (120, 121). In one set of experiments Fakhrai et al., used antisense to TGFβ to inhibit the expression of that cytokine in a rat gliosarcoma cell line. Immunization of tumor-bearing rats with the antisense modified tumor cells resulted in significant survival of animals compared to animal's immunization with tumor cells modified with control vectors. Using a different approach Isaka et al., was able reduce the amount fibrotic disease in rats, by transfecting skeletal muscle with a cDNA encoding decorin. Decorin is a small proteoglycan that inhibits the expression of TGFβ. Thus, two different approaches to inhibit TGFβ expression has shown efficacy in two different models of cancer or pre-cancer.

In addition, new evidence demonstrates that co-stimulation of T cells by B7 has both a positive and negative effect on T cell activation (122). Other co-stimulatory molecules for T cells such as ICAM-I, LFA-3 and VCAM-I have also been implicated in the induction of appropriate anti-tumor responses (123). A general consensus among those skilled in the art is that the most important of these co-stimulatory signals is provided by the interaction of CD28 on T cells with its primary ligands B7-1 (CD80) and B7-2 (CD86) on the surface of antigen presenting cells (124). In a variety of model systems tumor cells transfected with the B7 cDNA induced potent antitumor responses against both modified and unmodified tumor cells. CTLA-4, a molecule also expressed on T cells, binds B7-1 and B7-2 with much higher affinities than CD28. Results of several studies demonstrate that CTLA-4 acts as a negative regulator of T cell responsiveness, and raises the possibility that blocking the inhibition delivered by the CTLA-4-B7 interaction might augment the T cell response to tumor cells and enhance antitumor activity. Leach et al., demonstrated that injecting antibody to CTLA-4 resulted in the rejection of tumors including pre-established tumors in a mouse model (124). This demonstrates the care must be used in designing gene transfer experiments such that the desired effects are not masked by other potential deleterious effects.

The genetic basis of cancer includes abnormalities in oncogenes and/or tumor suppressor genes. Both types have been the targets of cancer gene therapy. Because the cancer-related defects of tumor suppressor genes are usually mutations or deletions, the strategy in tumor suppressor gene therapy thus far developed has been gene replacement therapy, in which a wild-type tumor suppressor gene is transferred into cancer cells to restore the normal function of the defective gene or induce tumoricidal effect (124). The human tumor suppressor genes that have been cloned and characterized include Rb, Wilms tumor (WT1), and neurofibromatosis (NF1), which are involved in pediatric cancers; adenomatosis polyposis coli (APC) and deleted in colon cancer (DCC), which contribute to colorectal cancer; and p53, which is found in mutated forms in a wide range of human cancers (for a review, see Ref. 125). Recently, two major events occurred in the area of identification of new tumor suppressor genes or cancer susceptibility genes. First, two highly related members of the cyclin-dependent kinase (cdk) inhibitor family, termed p16 (major tumor suppressor 1, MTS1) and p15 (MTS2), were isolated from the chromosomal region 9p21 (126-128). Second, a strong candidate for the breast and ovarian cancer susceptibility gene BRCA 1 was identified (129). While p16 was shown to be deleted or mutated in a wide range of cancer cell lines, p15 was shown to be a potential effector of TGF-β-induced cell cycle arrest (130). Among all of those tumor suppressor genes, the p53 gene is the one that has thus far been utilized for gene therapy of cancer (131).

Our current effort on gene therapy of cancer is to combine tumor suppresser gene and immunomodulation gene therapy of cancer with the introduction of other molecules such as, tumor antigens, MHC molecules, cell adhesion molecules and other immunomodulating factors. The followings are the general description of the two designs of the anticancer super-Ad vectors.

22.1 Construction the First Version of the Anti-Cancer Super-Ad Vectors

Several combinations of immune molecules and genes may be utilized in the constrcution of anti-cancer super Ad vectors. The mini-Ad vectors may carry of the multiple genes that function to suppress tumor growth or induce host anticancer immune responses. This type of vectors is called anticancer super-Ad vectors. The first version of the super-Ad vector will carry four double expression cassettes for human p53 cDNA, GFP marker gene, human IL2 cDNA, human GM-CSF cDNA, human B7-1 cDNA, human IL7 cDNA and human IL12 p35 and p40 cDNA. It also contains minimum sequence of left and right Ad5 ITR and Ad5 packaging sequence (total 660 bp) and about 18 kb genomic sequence of human a-fetoprotein gene to reach over 30 kb size (FIG. 53). Cassette 1 includes a CMV promoter, a Human p53 cDNA, an EMC-IRES, a GFP gene and a SV40 pA. Cassette 2 includes an EF promoter, a human GM-CSF cDNA, an EMC-IRES, a human IL 12 cDNA and a bovine growth hormone pA. Cassette 3 comprises an SV40 promoter, human B7-1 cDNA, an EMC-IRES, human IL7 cDNA and SV40 pA. Cassette 4 includes a tk promoter, a Human IL12 p35 cDNA, an EMC-IRES, a human IL12 p40 cDNA and a bovine growth hormone pA (FIG. 53).

22.2 Construction the Second Version of the Anti-Cancer Super-Ad Vectors

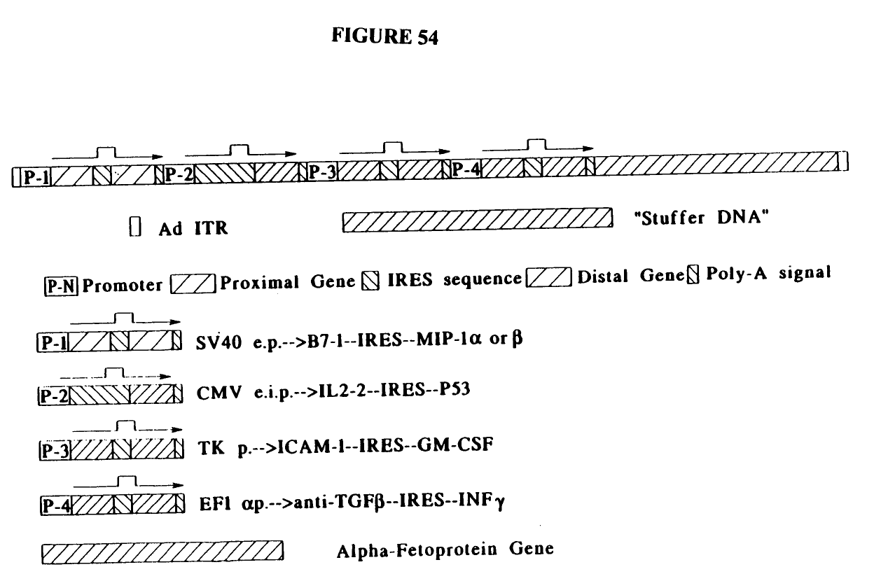

A second version of the anti-cancer super Ad vectors has a similar structure to that of the first version, including adenovirus inverted terminal repeats at both the 5′ and 3′ ends and four discrete expression cassettes. Several combinations of regulatory molecules and genes may be utilized in the constrcution of anti-cancer super Ad vectors. The examples described below are not in any way limiting to the types of mini-Ad vectors that may be constructed to regulate the growth of a tumor cell. Each expression cassette is flanked at the 5′ end by a unique promoter. In addition, each expression cassette incorporates two genes linked by the encephalomyocarditis virus internal ribosome entry site sequence for cap independent translation of the “distal” gene. The genes shown for this vector include cytokine genes as represented by IL-2, IL-7, and GM-CSF; a tumor suppresser gene as represented by p53; immune cell co-stimulatory molecules as represented by B7-1 and ICAM-1; and molecules that can reverse the immune suppression often associated with cancers, anti-TGFβ and SCA to CTLA-4. To increase the size of the vector so that the vector will be efficiently packaged into progeny virus, we have included a “stuffer DNA” of human alpha-fetoprotein. The stuffer DNA may include any DNA fragment of any length. The general structure of the second version of the anticancer super-Ad vectors are shown in FIG. 54.

### Example 23

### Other Designs for Improvement of the System

A. Mini-Ad Vectors Having Targeting Capability

Multiple mechanisms may be utilized to target gene expression to a specific cell type or tissue. One such mechanism involves transcriptional targeting of a cell type, cell type subset or a specific tissue. Transcriptional targeting includes the use of a transcriptional regulatory unit that drives gene expression in only a certain type of cell or tissue. Such a transcriptional regulatory unit is referred to as being tissue-specific. A mini-ad vector is designed to incorporate a tissue-specific transcriptional regulatory unit driving expression of a reporter or effector gene. In this manner, expression of the reporter or effector gene under control of the tissue-specific transcriptional regulatory unit will be detected at a higher level in those specific tissues in which the transcriptional regulatory unit is active. It may be preferable to restrict gene expression to a certain cell type or tissue. Therapeutic genes are often toxic if expressed in high amounts. Regulation of gene expression to specific tissues, then, may serve to protect the host from the adverse effects of high level gene expression of certain therapeutic genes.

A further method to direct tissue-specific gene expression would be to utilize a helper virus encoding a cell surface protein reactive to a ligand on a cell type of interest. For instance, a helper virus may be engineered to express a ligand for a cell surface receptor. Upon packaging of the recombinant packaging-competent DNA construct of this invention, an recombinant adenoviral particle that binds to a receptor on the surface of a cell is produced. A further example would include a recombinant adenovirus that expresses an antibody or a fragment of an antibody on the surface of its viral coat. Such a recombinant virus may be produced by engineering a packaging-deficient helper virus to express an antibody or antibody fragment as a fusion or a separate protein on its viral coat. Upon infection of a cell transfected with a DNA molecule encoding an at least an adenoviral packaging sequence and at least one reporter or effector gene, recombinant adenoviral particles having an antibody or antibody fragment reactive to a cell surface molecule on a target cell are produced. In this manner, recombinant adenoviral particles will specifically bind to those cells in the host that express cell surface molecules reactive to said antibodies or antibody fragments.

B. Mini-Ad Vectors for Local Immune Suppression

Certain autoimmune disorders result from the inappropriate immune reactions. One method that may be utilized to prevent, halt or slow the autoimmune reaction is to direct expression of immunomodulatory proteins at the site of such reactions. This may be accomplished by application of adenoviral particles constructed from a mini-Ad genome as demonstrated within this application. Genes encoding certain cytokines or chemokines may be expressed and such expression may result in an attenuation of the immune reponse. This attenuation in the immune response would then lead to an alleviation of the symptoms of the autoimmune reaction. A further example may include the attenuation of an allergic reaction. An antigen known to case an allergic reaction may be encoded by a mini-Ad vector. Upon expression either low levels or extremely high levels of the antigen, driven by the mini-Ad vector delivered to a cell by a recombinant adenoviral particle, tolerance may result. Also, expression of the antigen may be directed to tissues in which expression of the antigen may induce tolerance. Such a tissue may include the developing thymus. Following desensitization, the host into which the recombinant adenoviral particle was delivered will not exhibit an allergic reaction upon interaction with that antigen. In this manner, a form of immunosuppression has been achieved by administration of the recombinant adenoviral particle carrying engineered mini-Ad DNA molecule.

C. Other Strategies

It will also be possible to utilize the mini-Ad vectors disclosed in this application to prevent or eliminate viral infection and replication within a host. Mini-Ad vectors can be designed such that viral certain genetic processes may be interfered with or eliminated. The mini-Ad vectors may be designed to express antisense nucleic acids that interfere with viral replication at the transcriptional or translational stage of infection. Interference may be promoted by the expression of antisense RNA or DNA including that which binds to messenger RNA or binds to DNA after integration of a viral genome to prevent transcription. Also, ribozymes may be designed that target certain viral transcripts for destruction. “Decoy” molecules may also be encoded by a mini-Ad vector. Such decoys may function by binding to transcription factors required for viral trasncription such that the trasncription factors are no longer available for binding to and driving trasncription of genes required for viral gene expression and replication.

While a preferred form of the invention has been shown in the drawings and described, since variations in the preferred form will be apparent to those skilled in the art, the invention should not be construed as limited to the specific form shown and described, but instead is as set forth in the claims.

### REFERENCES

1. Mannuchi, P. M. 1993. Modern Treatment of Hemophilia: From the shadows towards the light. Thrombosis and Haemostasis 70: 17-23.

2. Kazazian, H. H. Jr. 1993. The molecular basis of hemophilia A and the present status of carrier and antenatal diagnosis of the diseaase. Thrombosis and Haemostasis 70: 600-62.

3. Furie, B, S. A. Limentani, and C. G. Rosenfield. 1994. A practical guide to the evaluation and treatment of hemophilia. Blood 84: 3-9.

4. Jones, L. K., and E. G. D. Tuddenham. 1995. Gene therapy for hemophilias. Gene Ther. 2: 699-701.

5. Toole, J. J., J. L. Knopf, J. M. Wozney, L. A. Sultzman, L. L. Buecker, D. D. Pitman, R. J. Kaufman, E. Brown, C. B. Sheoemaker, E. C. Orr, G. W. Amphlett, B. Foster, M. L. Coe, G. J. Knutson, D. N. Fass, and R. M. Hewick. 1984. Molecular cloning of a cDNA encoding human antihemophilic factor. Nature 312: 342-347.

6. Wood, W. I., D. J. Capon, C. C. Simonsen, D. L. Eaaton, J. Gitschier, B. Keyt, P. H. Seeburg, D. H. Smith, P. Hollingshead, K. Wion, E. Delwart, E. G. D Tuddenharn, G. A. Vehar, and R. M. Lawn. 1984. Expression of active human factor VIII from recombinant DNA clones. Nature 312: 330-337.

7. Truett, M. A. R. Blacher. R. L. Burke, D. Caput, C. Chu, D. Dina, K. Hartog, C. H. Kuo, F. R. Masiarz, J. P. Merryweather, R. Najarian, C. Pachl, S. J. Potter, J. Puma, M. Quiroga, L. B. Rall, A Randolph, M. S. Urdea, P. Valenzuela, H. H. Dahl, J. Favalaro, J. Hansen, O. Nordfang, and M. Ezban. 1985. Characterization of the polypeptide composition of human factor VIII:C and the nucleotide sequence and expression of the human kidney cDNA. DNA 4: 333-349.

8. Toole, J. J., D. D. Pittman, E. C. Orr, P. Murtha, L. C. Wasley, and R. J. Kaufman. 1986. A large region (˜95 kDa) of human factor VIII is dispensable for in vitro procoagulant activity. Proc. Natl. Acad. Sci. U.S.A. 83: 5939-5942.

9. Koeberl, D. D., C. L. Halbert, A. Krumm and A. D. Miller. 1995. Sequences within the coding regions of clotting factor VIII and CFTR block transcriptional elongation. Human Gene Ther. 6: 469-479.

10. Hoeben, R. C., F. J. Fallaux, S. J. Cramer, D. J. M. vand den Wollenberg, H. van Ormondt, E. Briet, and A. J. van der Eb. 1995. Expression of the blood-clotting factor-VIII cDNA is repressed by a transcriptional silencer located in its coding region. Blood. 85: 2447-2454.

11. Dwarki, V. J., P. Belloni, T. Nijar, J. Smith, L. Couto, M. Rabier, S. Clift, A. Berns and L. Cohen. 1995. Gene therapy for hemophilia A: Production of therapeutic levels of human factor VIII in vivo in mice. Proc. Natl. Acad. Sci. USA 92: 1023-1027.

12. Chuah, M. K. L., T. Vandendriessche, and R. A. Mogan. 1995. Development and analysis of retroviral vectors expressing human factor VIII as a potential gene therapy for hemophilia A. Human Gene Ther. 6: 1363-1377.

13. Connelly, S., T. A. G. Smith, G. Dhir, J. M. Gardnewr, M. G. Mehaffey, K. S. Zaret, A. McClelland and M. Kaleko. 1995. In vivo gene delivery and expression of physiological levels of functional human factor VIII in mice. Human Gene Therapy 6: 185-193.

14. Connelly, S., J. M. Gardner, A. McClelland, and M. Kaleko. 1996. High-level tissue-specific expression of functional human factor VIII in mice. Hum. Gene Ther. 7:183-195.

15. Connelly, S. J. Mount, A. Mauser, J. Gardner, M. Kaleko, A. McClelland, and C. D. Lothrop Jr. 1996. Complete short-term correction of canine hemophilia A by in vivo gene therapy. Blood 88: 3846-3853.

16. Connelly, S., J. M. Gardner, A., R. M. Lyons, A. McClelland, and M. Koleko. 1996. Sustained expression of therapeutic levels of human factor VIII in mice. Blood 87: 4671-4677.

17. Gluzman, Y. and K. Van Doren. 1983. Palindromic adenovirus type 5-simian virus 40 hybrid. J. Viol. 45: 91-103.

18. Grable, M., and P. Hearing. 1992. cis and trans requirements for the selective packaging of adenovirus type 5 DNA. J. Virol. 66: 723-731.

19. Fisher, K. J., H. Choi, J. Burda, S-J Chen, and J. M. Wilson. 1996 Recombinant adenovirus deleted of all viral genes for gene therapy of cystic fibrosis. Virology 217: 11-22.

20. Haecker, S. E., H. H. Stedman, R. J. Balice-Gordon, D. B. J. Smith, J. P. Greelish, M. A. Mitchell, A. Wells, H. L. Sweeney, and J. M. Wilson. 1996. In vivo expression of full-length human dystrophin from adenoviral vectors deleted of all viral genes. Hum. Gene Ther. 7: 1907-1904.

21. Kochanek, S., P. R. Clemens, K. Mitani, H-H Chen, S. Chan, and C. T. Caskey. 1996. A new adenoviral vector: Replacement of all viral coding sequences with 28 kb of DNA independently expressing both full-length dystrophin and β-galactosidase. Proc. Natl. Acad. Sci. USA 93: 5731-5736

22. Kotin, R., Siniscalo, M., Samulski, J., Zhu, X., Hunter, L., Laughlin, C., McLaughlin, S., Muzyczka, N., Rocchi, M., and Berns, K. I. (1990) Site-specific integration by adeno-associated virus. Proc. Natl. Acad. Sci. USA 87: 2211-2215.

23. Samulski, J., Zhu, X., Brook, J., Housman, D., Epstein, N., and Hunter, L. (1991) Targeted integration of adeno-associated virus (AAV) into human chromosome 19. EMBO J. 10: 3941-3950.

24. Shelling, A. and Smith, M. (1994) Targeted integration of transfected and infected adeno-associated virus vectors containing the neomycin resistance gene. Gene Therapy 1: 165-169.

25. Giraud, C., E. Winocour, and K. I. Berns 1994. Site-specific integration by adeno-associated virus is directed by a cellular sequence. Proc. Natl. Acad. Sci. USA 91: 10039-10043.

26. Giraud, C., E. Winocour, and K. Berns. (1995) Recombinant junctions formed by site-specific integration of adeno-associated virus into an episome. J. Virol. 69: 6917-6924.

27. Linden, R., E/Winocour, and K. Berns. (1996) The recombination signals for adeno-associated virus site-specific integration. P.N.A.S. 93: 7966-7972.

28. Urcelay, E., P. S. M. Ward, S. M. Wiener, B. Safer, and R. Kotin 1995. Asymmetric replication in vitro from a human sequence element is dependent on adeno-associated virus rep protein. J. Virol. 69: 2038-2046.

29. Halbert, C., L. Alexander, G. Wolgamot, and D. Miller. (1995) Adeno-associated virus vectors transduce primary cells much less efficiently than immortalized cells. J. of Virology 69: 1473-1479.

30. Tripathy, S., H. Black, E. Goldwasser, and J. Leiden. (1996). Immune responses to transgene-encoded proteins limit the stability of gene expression after injection of replication-defective adenovirus vectors. Nature Medicine 2: 545-550.

31. Ghosh-Choudhury, G., Y. Haj-Ahmad, and F. L. Graham. 1987. Protein IX, a minor component of the human adenovirus capsid, is essential for the packaging of full-length genomes. EMBO J. 6: 1733-1739.

32. Hayashi, Y., J. Chan, H. Nakabayashi, T. Hasimoto, and T. Tamaoki. 1992. Identification and characterization of two Enhancers of the human albumin gene. J. Biol. Chem. 267: 14580-14585.

33. Urano, Y., M. Sakai, K. Watanabe, and T. Tamaoki. 1984. Tandem arrangement of the albumin and alpha-fetoprotein genes in the human genome. Gene 32: 255-261.

34. Berns, K. I. 1996. Parvoridiae: the viruses and their replication. Fields Virology. Philadelphia, Lippincot-Raven. Third, ed. 2173-2197.

35. Kotin, R. M., R. M. Linden, and K. I. Berns 1992. Characterization of a preferred site on human chromosome 19q for integration of adeno-associated virus DNA by non-homologous recombination. EMBO J. 11: 5071-5078.

36. Im, D. S., and N. Muzyczka 1989. Factors that bind to adeno-associated virus terminal repeats. J. Virol. 63: 3095-3104.

37. Ashktorab, H., and A. Srivastava 1989. Identification of nuclear proteins that specifically interact with adeno-associated virus type 2 inverted terminal repeat hairpin DNA J. Virol. 63: 3034-3039.

38. Im, D. S., and N. Muzyczka 1990. The AAV origin binding protein Rep68 is an ATP-dependent site-specific endonuclease with DNA helicase activity. Cell 61: 447-457.

39. Im, D. S., and N. Muzyczka 1992. Partial purification of adeno-associated virus Rep78, Rep68, Rep52 and Rep40 and their biochemical characterization. J. Virol. 66: 1119-1128.

40. Wonderling, R. S., S. R. M. Kyöstiö, and R. A. Owens 1995. A maltose-binding protein/adeno-associated virus rep68 fusion protein has DNA-RNA helicase and ATPase activities. J. Virol. 69: 3542-3548.

41. Weitzman, M. D., S. R. M. Kyöstiö, R. M. Kotin, and R. A. Owens 1994. Adeno-associated virus (AAV) rep proteins mediate complex formation between AAV DNA and its integration site in human DNA. Proc. Natl. Acad. Sci. USA 91: 5808-5812.

42. Deuschle, U., W. K-H Meyer, and H-J Thiesen. 1995. Tetracycline-reversible silencing of eukaryotic promoters. Mol. Cell. Biol. 15: 1907-1914.

43. Robertson, E. J. 1987. Teratocarcinomas and embryonic stem cells: A practical Approach. IRL Press, Oxford. pp. 71-182.

44. Pittman, D. D., E. M. Alderman, K. N. Tomkinson, J. H. Wang, A. R. Giles, and R. J. Kaufman. 1993. Biochemical, Immunological, and in vivo characterization of B-domain-deleted factor VIII. Blood 81: 2925-2935.

45. Ghebranious, N., B. J. Knoll, L. Yavorkovsky, Z. Ilic, J. Papaconstantinou, G. Lozano, and S. Sell. 1995. Developmental control of transcription of the CAT reporter gene by a truncated mouse alphafetoprotein gene regulatory region in transgenic mice. Ma. Reprod. Dev. 42: 1-6.

46. Spear, B. T., and S. M. Tilghman. 1990. Role of alpha-fetoprotein regulatory elements in transcriptional activation in transient heterokaryons. Mol. Cell. Biol. 10: 5047-5054.

47. Feuerman, M. H., R. Godbout, R. S. Ingram, and S. M. Tilghman. 1989. Tissue-specific transcription of the mouse alpha-fetoprotein gene promoter is dependent on HNF-1. Mol. Cell. Biol. 9: 4204-4212.

48. Vogt, T. F., R. S. Compton, R. W. Scott, and S. Tilghman. 1988. Differential requirements for cellular enhancers in stem and differentiated cells. Nucleic Acids Res. 16: 487-500.

49. Mittereder, N., K. L. March, and B. C. Trapnell. 1996. Evaluation of the concentration and bioactivity of adenovirus vectors for gene therapy. J. Viol. 70: 7498-7509.

50. Kotin, K., R. Linden, and K. Berns. 1992. Characterization of a preferred site on human chromosome 19q for integration of adeno-associated virus DNA by non-homologous recombination. EMBO J. 11: 5071-5078.

51. Bi, L., A. Lawler, S. Antonarakis, K. High, J. Gearhart, and H. Kazazian. 1995. Targeted disruption of the mouse factor VIII gene produces a model of Hemophilia A. Nature Genetics 10: 119-121.

52. Mizushimia and Nagata. 1990. pEF-BOS, a powerful mammalian expression vector. Nuc. Acids Res. 18:5322.

53. Fay, P. J. 1993. FVIII structure and function. Thromb. Haemostas. 70: 63-67.

54. McGrory, et al. 1988. A simple technique for rescue of early region I mutations into infectious human adenovirus type 5. Virology, 163: 614-617.

55. “The Metabolic and Molecular Bases of Inherited Diseases” (Editors: C. R. Scriver, A. L. Beaudet, W. S. Sly, and D. Valle, 7th edition, 1996).

56. Balague, C., Kalla, M. an Zhang, W-W. (1997) Adeno-associated Rep78 protein and terminal repeats enhance integration of DNA sequences into the cellular genome. J. Virol. 71: 3299-3306.

57. Gendron-Maguire, M. and T. Gridley. 1993. Identification of transgenic mice. Methods in Enzymology 225: 794-799.

58. Mann, J. R. 1993. Surgical techniques in production of transgenic mice. Methods in Enzymology 225: 782-793.

59. Gordon, J. W. 1993. Production of transgenic mice. Methods in Enzymology 225: 747-771.

60. Mann, J. R. and A. P. McMahon. 1993. Factors influencing frequency production of transgenic mice.

61. Bradley, A. 1987. Production and analysis of chimaeric mice. In Teratocarcinomas and embryonic stem cells—a practical approach. Pp. 113-151. Robertson, E. J., Ed. IRL Press, Washington, D.C.

62. Koeberl, et al. 1995. Sequences within the coding regions of clotting factor VIII and CFTR block transcriptional elongation. Human Gene Ther. 6: 469-479.

63. Hoeben, et al. 1995. Expression of the blood-clotting factor FVIII cDNA is repressed by a transcriptional silencer located in its coding region. Blood 85:2447-2454.

64. Miller, A. D. and G. J. Rosman. 1989. Improved retroviral vectors for gene therapy and expression. Biotechniques 7: 980-990.

65. Mulligan, R. C. 1993. The basic science of gene therapy. Science 260: 926-932.

66. Parks, et al. 1996. A helper-dependent adenovirus helper system: removal of helper virus by cre-mediated excision of the viral packaging signal. Proc. Natl. Acad. Sci. USA 93: 13565-13570.

67. Hitt, et al. In Methods in Molecular Genetics, K. W. Adolph, ed. (Academic Press, San Diego, Calif.) Vol. 7, pp. 13-30.

68. Broach, et al. 1982. Recombination within the yeast plasmid 2mu circle is site-specific. Cell 29: 227-234.

69. Gorman, et al. 1982. Recombinant genomes which express chloramphenicol acetyltransferase in mammalian cells. Mol. Cell. Biol. 2: 1044-1051.

70. Calos, M. P. 1996. The potential of extrachromosomal replicating vectors for gene therapy. TIG 12: 463-466.

71. Krysan, et al. 1993. Autonomous replication in human cells of multimers of specific human and bacterial DNA sequences. Mol. Cell. Biol. 13: 2688-2696.

72. Haase, S. B. and M. P. Calos. 1991. Replication control of autonomously replicating human sequences. Nucleic Acids Res. 19: 5053-5058.

73. Kay, et al. 1993. In vivo gene therapy of hemophilia B: sustained partial correction in factor IX-deficient dogs. Science 262: 117-119.

74. Chuah, et al. 1995. Development and analysis of retroviral vectors expressing human factor VIII as a potential gene therapy for hemophilia A. Human Gene Ther. 6: 1363-1377.

75. Hammarskjold, M.-L. and G. Winberg. 1980. Encapsidation of adenovirus 16 DNA is directed by a small DNA sequence at the left end of the genome. Cell 20: 787-795.

76. Yang, Y., et al. 1994. Cellular immunity to viral antigen limits E1-deleted adenoviruses for gene therapy. Proc. Natl. Acad. Sci. U.S.A. 91: 4407-4411.

77. Dwarki, V. J., et al. 1995. Gene therapy for hemophilia A: Production of therapeutic levels of human factor VIII in vivo in mice. Proc. Natl. Acad. Sci. USA 92: 1023-1027.

78. Dai, et al. 1992. Gene therapy via primary myoblasts: Long-term expression of factor IX protein following transplantation in vivo. Proc. Natl. Acad. Sci. USA 89: 10892-10895.

79. Cotton, M., et al. 1992. High-efficiency receptor-mediated delivery of small and large (48 kilobase) gene constructs using the endosome disruption activity of defective or chemically-inactivated adenovirus particles. Proc. Natl. Acad. Sci. USA 89: 6094-6098.

80. Frank, et al. 1994. High-level expression of various apolipoprotein(a) isoforms by “transferinfection”: the role of kringle IV sequences in the extracellular association with low-density lipoprotein. Biochemistry 3: 12329-12339.

81. Chen, C. and H. Okayama. 1987. High-efficieny transformation of mammalian cells by plasmid DNA. Mol. Cell. Biol. 7: 2745-2752.

82. Marchioro, T. L., et al. 1969. Science 163:188.

83. Webster, et al. 1971. Am. J. Physiology 220:1147.

84. Graham, F. L., and L. Prevec. 1991. Manipulation of adenovirus vectors. In: Methods in Molecular Biology (Vol. 7), Gene Transfer and Expression Protocols, ed. E. J. Murray, The Humana Press Inc., Clifton, N.J.

85. Engelhardt, J. F., X. Ye, B. Doranz, and J. M. Wilson. 1994. Ablation of E2A in recombinant adenoviruses improves transgene persistence and decreases inflammatory response in mouse liver. Proc. Natl. Acad. Sci. 91: 6196-6200.

86. Krouglik, V., and F. L. Graham. 1995. Development of cell lines capable of complementing E1, E4, and protein IX defective adenovirus type 5 mutants. Human Gene Ther. 6: 1575-1586.

87. Mitani, K., F. L. Graham, C. T. Caskey, and S. Kochanek. 1995. Rescue, propagation, and patial purification of a helper virus-dependent adenovirus vector. Proc. Natl. Acad. Sci. 92: 3854-3858.

88. Lieber, et al. 1996. Recombinant adenoviruses with large deletions generated by Cre-mediated excision exhibit different biological properties compared with first-generation vectors in vitro and in vivo. J. Virology 70:894-8960.

89. Stow, N. D. 1981. Cloning of a DNA fragment from the bleft-hand terminus of the adenoviruse type 2 genome and its use in site-directed mutagenesis J. Virol. 37, 171-]80.

90. Grable, M., and P. Hearing. 1992. cis and trans requirements for the selective packaging of adenovirus type 5 DNA. J. Virol. 66: 723-731.

91. Graham, F. L., and L. Prevec. 1991. Manipulation of adenovirus vectors. In: Methods in Molecular Biology (Vol. 7), Gene Transfer and Expression Protocols, ed. E. J. Murray, The Humana Press Inc., Clifton, N.J.

92. Graham, F. L. 1984. Covalently closed circles of human adenovirus DNA. EMBO J. 3: 2917-2922.

93. Ruben, M., S. Bacchetti, and F. L. Graham 1983. Nature 301: 172-174.

94. McGrory, W. J., D. S. Bautista, and F. L. Graham 1988A simple technique for the rescue of early region I mutations into infectious human adenovirus type 5. Viology 163: 614-617.

95. Robinson, A. J., H. B. Younghusband, and A. J. D. Bellett. 1973. A circular DNA-protein complex from adenovirus. Virol. 56: 54

96. Bett, A. J., W. Haddara, L. Prevec, and F. L. Graham 1994. An efficient and flexible system for construction of adenovirus vectors with insertions or deletions in early regions 1 and 3. Proc. Natl. Acad. Sci. USA 91: 8802-8806.

97. Grable, M., and P. Hearing. 1990. Adenovirus type 5 packaging domain is composed of a repeated element that is functionally redundant. J. Virol. 64: 2047-2056.

98. Webster, N., J. R. Jin, S. Green, M. Hollis, and P. Chambon 1988. The yeast UAS_{G} is a transcriptional enhancer in human Hela cells in the presence of the Gal4 trans-activator. Cell 52: 169-178,

99. Gatz, C. and P. H. Ouau 1988. Tn10-encodes tet repressor can regulate an operator-containing plant promoter. Proc. Natl. Acad. Sci. USA 85: 1394-1397.

100. Gossen, M. and H. Bujard 1992. Tight control of gene expression in mammalian cells by tetracycline-responsive promoters. Proc. Natl. Acad. Sci. USA 89: 5547-5551.

101. Nevins, J. R. 1993. Transcriptional activation by the adenovirus E1A proteins. Semin. Viol. 4: 25-31.

102. Graham, F. L., P. J. Abrahams, C. Mulder, H. I. Heijneker, S. O. Warnaar, F. A. J. de Vries, W. Fiers, and A. J. van der Eb 1974. Studies on In Vitro Transformation by DNA and DNA Fragments of Human Adenoviruses and Simian Virus 40. Cold Spring Harbor Symp. Quant. Biol. 39: 637-650.

103. Mckinnon, R. D., S. Bacchetti, and F. L. Graham 1982. Tn5 Mutagenesis of the Transforming Genes of Human Adenovirus Type 5. Gene 19: 33-42.

104. van der Eb, A. J., C. Mulder, F. L. Graham, and A. Houweling 1977. Transformation With Specific Fragments of Adenovirus DNAs. I. Isolation of Specific Fragments With Transforming Activity of Adenovirus 2 and 5 DNA. Gene 2: 115-132.

105. Graham, F. L., J. Smiley, W. C. Russell, and R. Nairn. 1977. Characteristics of a human cell line transformed by DNA from human adenovirus type 5. J. Gen. Virol. 36: 59-72.

106. Aiello, L., R. Guilfoyle, K. Huebner, and R. Weinmann 1979. Adenovirus 5 DNA Sequences Present and RNA Sequences Transcribed in Transformed Human Embryo Kidney Cells (HEK-Ad-5 or 293). Virology 94: 460-469.

107. Lochmuller, H., A. Jani, J. Huard, S. Prescott, M. Simoneau, B. Massie, G. Karpati, and G. Ascadi, G. 1994. Emergence of Early Region 1-Containing, Replication-Competent Adenovirus in Stocks of Replication Defective Adenovirus Recombinants (E1+E3 Deletions) During Multiple Passages in 293 Cells. Human Gene Ther. 5: 1485-1491.

108. Fallaux, F. J., O. Kranenburg, S. J. Cramer, A. Houweling, H. van Ormondt, R. C. Hoeben, and A. J. van der Eb 1996. Characterization of 911: A new Helper Cell Line for the Titration and Propagation of Early Region 1-Deleted Adenoviral Vectors. Human Gene Ther. 7: 215-222.

109. Imler, J-L., C. Chartier, D. Dreyer, A. Dieterle, M. Sainte-Marie, T. Faure, A. Pavirani, and M. Mehtali 1996. Novel Complementation Cell Lines Derived From Human Lung Carcinoma A549 Cells Support the Growth of E1-Deleted Adenovirus Vectors. Gene Ther. 3: 75-84.

110. Miller, S. A., D. D. Dykes, and H. F. Polesky 1988. A simple salting out procedure for extracting DNA from human nucleated cells. Nucl. Acid. Res. 16: 1215.

111. Kilby, N. J., M R. Snaith, and J. A. H. Murray. 1993. Site-specific recombinases: tools for genome engineering. Trends Genet. 9: 413-421.

112. Austin, S., M. Ziese, and N. Sternberg. 1981. A novel role for site-specific recombination in maintenance of bacterial replicons. Cell 25: 729-36.

113. Broach J. R., V. R. Guarascio, and M. Jayaram. 1982. Recombination within the yeast plasmid 2mu circle is site-specific. Cell 29: 227-34.

114. Wobbe C. R., F. Dean, L. Weissbach, and J. Hurwitz. 1985. In vitro replication of duplex circular DNA containing the simian virus 40 DNA origin site. Proc. Natl. Acad. Sci. USA, 82: 5710-4.

115. Yates J. L., N. Warren, and B. Sugden. 1985. Stable replication of plasmids derived from Epstein-Barr virus in various mammalian cells. Nature 313: 812-5.

116. Yates, J. L., and N. Guan. 1991. Epstein-Barr Virus-derived plasmids replicate only once per cell cycle and are not amplified after entry into cells. J. Virol. 65: 483-488.

117. Miller, A. R., W. H. McBride, K. Hunt, and J. S. Economou. 1994. Cytokine-mediated gene therapy for cancer. Ann. of Surg. Onc. 1:436-450.

118. Rosenberg, S. A., M. T. Lotze, J. C. Tang, P. M. Aebersold, W. M. Linehan, C. A. Deipp, and D. E. White. 1989. Experience with the use of high-dose interleukin-2 in the treatment of 652 cancer patients. Ann. Surg. 210:474-484.

119. Dranoff, G. and R. C. Mulligan. 1995. Gene transfer as cancer therapy. Adv. in Immunol., 58:417-454.

120. Isaka, Y., D. K. Brees, K. Ikegaya, T. Kaneda, E. Irnai, N. A. Noble, And W. A. Border. 1996. Gene therapy by skeletal muscle expression of decorin prevents fibrotic disease in rat kidney. Nat. Med. 1996, 2:418-423.

121. Fakhrai, H., O. Dorigo, D. L. Shawler, H. Lin, D. Mercola, K. L. Black, I. Royston, and R. E. Sobol. 1996 Eradication of established intracranial rat gliomas by transforming growth factor b antisense gene therapy. Proc. Natl. Acad. Sci. U.S.A. 93:2909-2914.

122. Leach, D. R., M. F. Krummel and J. P. Allison. 1996. Enhancement of antitumor immunity by CTLA-4 blockade. Science 271:1734-1736.

123. Springer, T. A. 1990. Adhesion receptors of the immune system. Nature. 346:425-434.

124. Lenschow, D. J., T. L. Walunas, and J. A. Bluestone. 1996. CD28/B7 system of T cell costimulation. Annu. Rev. Immunol. 14: 233-58.

125. Zhang, W.-W. 1995. Adenovirus as a system for delivering and expressing tumor suppressor genes in tumor cells. Method: A Companion to Methods in Enzymology 8: 198-214.

126. Harris, H. 1993. How tumor suppressor genes were discovered. FASEB J. 7: 978-979.

127. Kamb, A., N. A. Gruis, J. Weaver-Feldhaus, Q. Liu, K. Harshman, S. V. Tavtigian, E. Stockert, R. S. Day III, B. E. Johnson, and M. H. Skolnick. 1994. A cell cycle regulator potentially involved in genesis of many tumor types. Science 264: 436-440.

128. Nobori, T., K. Miura, D. J. Wu, A. Lois, K. Takabayashi, and D. A. Carson. 1994. Deletions of the cyclin-dependent kinase-4 inhibitor gene in multiple human cancers. Nature 368: 753-756.

129. Miki, Y., J. Swensen, D. Shattuck-Eidens, P. A. Futreal, K. Harshman, S. Tavtigian, Q. Liu, C. Cochran, L. M. Bennett, W. Ding, R. Bell, J. Rosenthal, C. Hussey, T. Tran, M. McClure, C. Frye, T. Hattier, R. Phelps, A. Haugen-Strano, H. Katcher, K. Yakumo, Z. Gholami, D. Shaffer, S. Stone, S. Bayer, C. Wray, R. Bogden, P. Dayananth, J. Ward, P. Tonin, S. Narod, P. K. Bristow, F. H. Norris, L. Helvering, P. Morrison, P. Rosteck, M. Lai, J. C. Barrett, C. Lewis, S. Neuhausen, L. Cannon-Albright, D. Goldgar, R. Wiseman, A. Kamb, and M. H. Skolnick. 1994. A strong candidate for the breast and ovarian cancer susceptibility gene BRCA1. Science 266: 66-71.

130. Hannon G., and D. Beach. 1994. p15INK4B is a potential effector of TGF-β-induced cell cycle arrest. Nature 371: 257-261.

131. Zhang, W.-W., and X. Fang. 1995. Gene therapy strategies for cancer. Exp. Opin. Invest. Drugs 4: 487-514.

132. International Patent Application number PCT/US96/05310, published Oct. 24, 1996 as WO 96/33280.

133. International Patent Application numver PCT/US96/14312, published Mar. 13, 1997 as WO 97/09442.

134. International Patent Application number PCT/GB96/02061, published Mar. 6, 1997 as WO 97/08330.

What is claimed is:

1. A method of treating a condition in a mammal, comprising:
a. providing a recombinant adenovirus comprising a nucleotide sequence comprising an adenoviral inverted terminal repeat fusion sequence, a packaging signal, a transcriptional control region, and a nucleic acid encoding a therapeutic protein, wherein the remaining portion of said DNA molecule does not encode an adenoviral protein; and,
b. administering said recombinant adenovirus to a mammal under conditions that result in the expression of the therapeutic protein at a level that provides a therapeutic effect in said mammal.

2. The method of claim 1 wherein said therapeutic protein is FVIII.

3. The method of claim 2 wherein said therapeutic protein is human FVIII.

4. The method of claim 1, wherein said therapeutic protein is expressed in the liver.

5. The method of claim 4 wherein said therapeutic protein is FVIII.

6. The method of claim 5 wherein said therapeutic protein is human FVIII.

7. The method of claim 7 wherein said recombinant adenovirus is FVIII.

8. The method of claim 8 wherein said therapeutic protein is human FVIII.

9. The method of claim 1, wherein the nucleic acid encoding the therapeutic protein is under the transcriptional control of a promoter.

10. The method of claim 1, wherein the nucleic acid encoding the therapeutic protein is under the transcriptional control of a tissue-specific promoter.

11. The method of claim 10, wherein said promoter is a liver-specific promoter.

12. The method of claim 11, wherein said promoter is an albumin promoter.

13. The method of claim 11, wherein said promoter is an alpha-fetoprotein promoter.

14. The method of claim 1 wherein the nucleotide sequence of said recombinant adenovirus further comprises an additional segment of cellular DNA.

15. The method of claim 14 wherein said additional segment of cellular DNA is a fragment of a gene.

16. The method of claim 15 wherein said additional segment of cellular DNA is a fragment of the albumin gene.

17. The method of claim 1, wherein said therapeutic protein is FVIII and the nucleic acid encoding FVIII is under the transcriptional control of a promoter.

18. The method of claim 1, wherein said therapeutic protein is FVIII and the nucleic acid encoding FVIII is under the transcriptional control of a tissue-specific promoter.

19. The method of claim 18, wherein said therapeutic protein is FVIII and the nucleic acid encoding FVIII is under the transcriptional control of a liver-specific promoter.

20. The method of claim 19, wherein said therapeutic protein is FVIII and the nucleic acid encoding FVIII is under the transcriptional control of an albumin promoter.

21. The method of claim 19, wherein said therapeutic protein is FVIII and the nucleic acid encoding FVIII is under the transcriptional control of an alpha-fetoprotein promoter.

22. The method of claim 17, wherein said therapeutic protein is FVIII, the nucleic acid encoding FVIII is under the transcriptional control of a promoter, and the nucleotide sequence of the recombinant adenovirus comprises an additional segment of cellular DNA.

23. The method of claim 22, wherein said therapeutic protein is FVIII, the nucleic acid encoding FVIII is under the transcriptional control of a tissue-specific promoter, and the nucleotide sequence of the recombinant adenovirus comprises an additional segment of cellular DNA.

24. The method of claim 23, wherein said therapeutic protein is FVIII, the nucleic acid encoding FVIII is under the transcriptional control of a liver-specific promoter, and the nucleotide sequence of the recombinant adenovirus comprises an additional segment of cellular DNA.

25. The method of claim 24, wherein said therapeutic protein is FVIII, the nucleic acid encoding FVIII is under the transcriptional control of an albumin promoter, and the nucleotide sequence of the recombinant adenovirus comprises an additional segment of cellular DNA.

26. The method of claim 24, wherein said therapeutic protein is FVIII, the nucleic acid encoding FVIII is under the transcriptional control of an alpha-fetoprotein promoter, and the nucleotide sequence of the recombinant adenovirus comprises an additional segment of cellular DNA.

27. The method selected from the group consisting of the method of claim 22, 23, 24, 25, and 26 wherein said additional segment of cellular DNA is a fragment of the albumin gene.

28. The method of claim 1, wherein said recombinant adenovirus is administered by a route selected from the group consisting of oral, parenteral, inhalation, rectal, topical, intravenous, intrarterial, intrapleural, nasal, intrathecaly, and direct intaorgan injection.

29. The method of claim 17, wherein said recombinant adenovirus is administered by a route selected from the group consisting of oral, parenteral, inhalation, rectal, topical, intravenous, intrarterial, intrapleural, nasal, intrathecaly, and direct intaorgan injection.
